(19) United States
(12) Patent Application Publication
(10) Pub. No.: US 2019/0355438 A1
(43) Pub. Date: Nov. 21, 2019

(54) INFERRING SELECTION IN WHITE BLOOD CELL MATCHED CELL-FREE DNA VARIANTS AND/OR IN RNA VARIANTS

(71) Applicant: GRAIL, INC., Menlo Park, CA (US)

(72) Inventors: Oliver Claude Venn, San Francisco, CA (US); Earl Hubbell, Palo Alto, CA (US)

(73) Assignee: GRAIL, INC., Menlo Park, CA (US)

(21) Appl. No.: 16/417,336

(22) Filed: May 20, 2019

Related U.S. Application Data
(60) Provisional application No. 62/673,779, filed on May 18, 2018.

(51) Int. Cl. G16B 20/20; C12Q 1/6874; C12N 15/10; G16B 30/10

(52) U.S. Cl. CPC G16B 20/20; G16B 30/10; C12N 15/1089; C12Q 1/6874

(57) ABSTRACT

Methods and systems for detecting positive, neutral, or negative selection at a locus include obtaining a test sample of cell-free nucleic acids from a subject, preparing a sequencing library of the cell-free nucleic acids, sequencing the library to obtain a plurality of sequence reads, analyzing the sequence reads to detect and quantify one or more somatic mutations at the locus, determining a selection coefficient for the locus, and comparing the selection coefficient with a threshold value to detect positive, neutral, or negative selection at the locus.

### CROSS REFERENCE TO RELATED APPLICATIONS

This application claims the benefit of priority to U.S. Provisional Patent Application No. 62/673,779, filed on May 18, 2018, and entitled “INFERRING SELECTION IN WHITE BLOOD CELL MATCHED CELL-FREE DNA VARIANTS AND/OR IN RNA VARIANTS,” the contents of which are herein incorporated by reference in their entirety.

### TECHNICAL FIELD

This application is generally directed to assessing biological samples, and more specifically, to assessing variants in biological samples.

### BACKGROUND OF THE INVENTION

Hematopoietic stem cells (HSCs) and hematopoietic progenitor cells (HPCs) divide to produce blood cells by a continuous regeneration process. As the cells divide, they are prone to accumulating mutations that generally do not affect function. However, some mutations confer advantages in self-renewal, proliferation or both, resulting in clonal expansion of the cells comprising the mutations in question. Although these mutations are not necessarily carcinogenic, the accumulation of mutations during clonal expansion can, eventually, lead to a carcinogenic phenotype. The frequency of such events appears to increase with age.

It has been observed that mutations in certain genes are associated with proliferating somatic clones, such as DNMT3A, TET2, JAK2, ASXL1, TP53, GNAS, PPM1D, BCORL1 and SF3B1 (Xie et al., Nature Medicine, published online 19 Oct. 2014; doi:10.1038/nm.3733). However, the relationship between the presence of clones comprising disruptive mutations in these genes have only been identified in 5-10% of human subjects over 65 years of age (see, e.g., Jaiswal et al.. The New England Journal of Medicine 2014 (overall 4.3% CHIP incidence that increases with age and predisposes to heme malignancies, cardiovascular events, and mortality); and Genovese et al.. The New England Journal of Medicine 2014 (overall 10% incidence in pts>65 yrs, 1% in pts younger than 50 yrs)). The influence of non-disruptive mutations has not been separately analyzed.

Several lines of evidence have suggested that clonal hematopoiesis due to an expansion of cells harboring an initiating driver mutation might be an aspect of the aging hematopoietic system. Clonal hematopoiesis in the elderly was first demonstrated in studies that found that approximately 25% of healthy women over the age of 65 have a skewed pattern of X-chromosome inactivation in peripheral blood cells (Busque I, et al. Blood 1996; and Champion K M et al. British journal of haematology 1997) which in some cases is associated with mutations in TET2 (Busque L et al. Nature genetics 2012). Large-scale somatic events such as chromosomal insertions and deletions and loss of heterozygosity (LOH) also occur in the blood of ˜2% of individuals over the age of 75 (Jacobs K B et al. Nature genetics 2012; and Laurie C C et al. Nature genetics 2012). Pre-leukemic hematopoietic stem cells (HSCs) harboring only the initiating driver mutation have been found in the bone marrow of patients with AML in remission (Jan M et al. Science translational medicine 2012; and Shlush L I et al. Nature 2014).

Recent sequencing studies have identified a set of recurrent mutations in several types of hematological malignancies (see, e.g., Mardis E R et al. The New England Journal of Medicine 2009; Bejar R et al. The New England Journal of Medicine 2011; Papaemmanuil E et al. The New England Journal of Medicine 2011; and Walter et al. Leukemia 2011). However, the frequency of these somatic mutations in the general population is unknown.

Accordingly, there is a need in the art for improved methods for detecting somatic mutations associated with clonal hematopoiesis and/or disease.

### BRIEF SUMMARY OF THE INVENTION

In general, the present invention is directed to methods and systems for assessing one or more somatic variants in a biological sample. In various embodiments, the present invention includes detecting positive, neutral, or negative selection at a locus, classifying a subject as having clonal hematopoiesis of indeterminate potential (CHIP), and assessing a disease state of a subject. In some embodiments, selection at a locus, CHIP, or disease state assessment is based on white blood cell (WBC) matched cell-free DNA variants in a biological fluid sample, such as a blood sample.

In some embodiments, the present invention is directed to a method for detecting positive, neutral, or negative selection at a locus, the method including: (a) obtaining a test sample from a subject, wherein the test sample includes a plurality of cell-free nucleic acids; (b) preparing a sequencing library from the plurality of cell-free nucleic acids; (c) sequencing the library to obtain a plurality of sequence reads, wherein the sequence reads are derived from the plurality of cell-free nucleic acids; (d) analyzing the plurality of sequence reads to detect and quantify one or more somatic mutations at the locus; (e) determining a selection coefficient for the locus; and (f) comparing the selection coefficient determined for the locus with a threshold value and detecting positive, neutral, or negative selection at the locus based on the comparison.

In some embodiments, a method for classifying a subject as having clonal hematopoiesis of indeterminate potential (CHIP) includes: (a) obtaining a test sample from a subject, wherein the test sample has a plurality of cell-free nucleic acids; (b) preparing a sequencing library from the plurality of cell-free nucleic acids; (c) sequencing the library to obtain a plurality of sequence reads, wherein the sequence reads are derived from the plurality of cell-free nucleic acids; (d) analyzing the plurality of sequence reads to detect and quantify one or more somatic mutation at a locus known to be associated with CHIP; and (e) classifying the subject as having CHIP when the one or more somatic mutations at the locus are detected.

In some embodiments, a method for assessing a disease state of a subject includes: at a first time point: (a) obtaining a first test sample from a subject at a first time point, the first test samples having a plurality of nucleic acids; (b) preparing a first sequencing library from the plurality of nucleic acids obtained from the first test sample; (c) sequencing the first sequencing library to obtain a plurality of sequence reads at a locus; (d) analyzing the plurality of sequence reads to detect and quantify one or more somatic mutations at the locus; (e) determining a first selection coefficient for the locus; at a second time point: (a) obtaining a second test sample from the subject at a second, the first and second test samples each having a plurality of nucleic acids; (b) preparing a second sequencing library from the plurality of nucleic acids from the second test sample; (c) sequencing the second sequencing library to obtain a plurality of sequence reads; (d) analyzing the plurality of sequence reads to detect and quantify one or more somatic mutations at the locus; determining a second selection coefficient for the locus; and comparing the first selection coefficient and the second selection coefficient, wherein an increase in the second selection coefficient relative to the first coefficient indicates progression in the disease state, and wherein a decrease in the second selection coefficient relative to the first selection coefficient indicates an improvement in the disease state.

In some aspects, the methods of the present invention further include: (a) obtaining white blood cells from the test sample; (b) isolating nucleic acids from the white blood cell and preparing a sequencing library from the white blood cell nucleic acids; (c) sequencing the library to obtain a plurality of sequence reads derived from the white blood cell nucleic acids; (d) analyzing the plurality of sequence reads derived from the white blood cell nucleic acids to detect and quantify one or more white blood cell derived somatic mutations; and (e) comparing the one or more cell-free nucleic acid detected somatic mutation and the one or more white blood cell derived somatic mutations to identify one or more white blood cell matched somatic mutations.

In some aspects, analyzing the plurality of sequence reads to detect and quantify the one or more somatic mutations further includes applying a noise model.

In some aspects, analyzing the plurality of sequence reads further includes applying a read mis-mapping model.

In some aspects, the one or more somatic mutations are detected from joint modeling or mixture modeling both the one or more cell-free nucleic acid somatic mutation and the one or more white blood cell derived somatic mutations.

In some aspects, the selection coefficient includes a ratio between the rate of non-synonymous substitutions per non-synonymous site and the rate of synonymous substitutions per synonymous site.

In some aspects, when the threshold is greater than 1 with a q-value less than 0.05, positive selection is detected.

In some aspects, when the threshold is less than 1 with a q-value less than 0.05, negative selection is detected.

In some aspects, the one or more somatic mutations include one or more single-nucleotide variants.

In some aspects, the one or more somatic mutations include one or more nonsynonymous mutations and the selection coefficient is determined based on the one or more nonsynonymous mutations.

In some aspects, the one or more somatic mutations include one or more missense mutations and the selection coefficient is determined based on the one or more missense mutations.

In some aspects, the one or more somatic mutations include one or more nonsense mutations and the selection coefficient is determined based on the one or more nonsense mutations.

In some aspects, the one or more somatic mutations include one or more truncating mutations and the selection coefficient is determined based on the one or more truncating mutations.

In some aspects, the one or more somatic mutations include one or more essential splice site mutations and the selection coefficient is determined based on the one or more essential splice site mutations.

In some aspects, the one or more somatic mutations are detected in an oncogene and the selection coefficient is determined for the oncogene.

In some aspects, the one or more somatic mutations are detected in a tumor suppressor gene and the selection coefficient is determined for the tumor suppressor gene.

In some aspects, the one or more somatic mutations include one or more insertions and/or deletions.

In some aspects, the one or more somatic mutations detected and quantified occur within one or more genes selected from the group consisting of DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B, and any combination thereof.

In some aspects, the detected and quantified somatic mutation occurs within the JAK2 gene.

In some aspects, the sequencing includes whole genome sequencing.

In some aspects, the sequencing includes whole exome sequencing.

In some aspects, the sequencing includes targeted sequencing.

In some aspects, the targeted sequencing further includes a targeted enrichment step prior to sequencing, and the targeted enrichment step includes an enrichment panel having from about 10 to bout 10,000 targeted genes, about 50 to bout 1,000 genes, or about 100 to bout 500 genes.

In some aspects, the sequencing library is sequenced to a depth of at least 100×, at least 500×, at least 1,000×, at least 2,000×, at least 3,000×, or at least 10,000×.

In some aspects, the selection coefficient determined at the locus is based on one or more somatic mutations detected at the loci have an allele frequency of from about 0.01% to about 35%, from about 0.01% to about 30%, or from about 0.01% to about 25%.

In some aspects, the selection coefficient determined at the locus is based on one or more somatic mutations detected at the loci have an allele frequency of less than about 10%, less than about 1%, less than about 0.1%, or less than about 0.01%.

In some aspects, the cell-free nucleic acids include cell-free DNA.

In some aspects, the somatic mutation is a ywhite blood cell matched mutation in cell-free DNA.

In some aspects, the cell-free nucleic acids include cell-free RNA.

In some aspects, the nucleic acids are DNA or RNA molecules isolated from one or more cells (e.g., white blood cells (WBCs)).

In some aspects, the test sample is a biological fluid sample.

In some aspects, the test sample is selected from the group consisting of a plasma, a serum, a urine, a cerebrospinal fluid, a fecal matter, a saliva, a pleural fluid, a pericardial fluid, a cervical swab, a saliva, a peritoneal fluid sample, and any combination thereof.

In some aspects, identification of the somatic mutation in a subject indicates that the subject will respond to a therapeutic treatment targeting the somatic mutation.

In some aspects, the method further includes assessing a risk of developing a disease state, detecting a disease state, and/or diagnosing a disease state based on the identification of one or more somatic mutations at the locus.

In some aspects, the disease state is a cardiovascular disease.

In some aspects, the cardiovascular disease is selected from the group consisting of atherosclerosis, myocardial infarction, atherosclerotic cardiovascular disease, atherosclerotic heart disease, atrial fibrillation, coronary artery disease, coronary heart disease, congestive heart failure, cerebrovascular accident/transient ischemic attack, heart failure, ischemic heart disease, venous thromboembolism, pulmonary embolism, and any combination thereof.

In some aspects, the disease state is cancer.

In some aspects, the cancer is acute leukemia, lymphoma, multiple myeloma, acute lymphocytic leukemia (ALL), acute myeloid leukemia (AML), chronic lymphocytic leukemia (CLL), chronic myelogenous leukemia (CML), Hodgkin's lymphoma, non-Hodgkin's lymphoma, or myelodysplastic syndrome.

In some aspects, the disease state is a neurodegenerative disease.

In some aspects, the locus detected as having positive selection is identified as a target for a therapeutic treatment.

In some aspects, the one or more detected somatic mutations are at locus targeted by immuno oncology therapy, targeted therapy, or synthetic lethality therapy.

In some aspects, the detection of positive, neutral, or negative selection is after therapeutic treatment with the immuno oncology therapy, targeted therapy, or synthetic lethality therapy.

In some aspects, the detection of negative selection after therapeutic treatment with treatment with the immuno oncology therapy, targeted therapy, or synthetic lethality therapy is an indicator of treatment response.

In some aspects, the targets of negative selection in patients under therapeutic treatment in a database of patients, optionally including I-MA type, are used to predict treatment response, and/or recommend treatment, and/or prioritize targeted immuno oncology therapy for a new patient optionally conditioning on their type.

In some aspects, the detection of positive selection under treatment with immuno oncology therapy, targeted therapy, or synthetic lethality therapy in an indicator of treatment response is used to identify resistance mechanisms to treatment.

Still, in some embodiments, the present invention is directed to a computer-implemented method for detecting positive, neutral, or negative selection at a locus, the method including: receiving a first data set in a computer having a processor and a computer-readable medium, wherein the first data set includes a plurality of sequence reads obtained by sequencing a plurality of cell-free nucleic acids in a test sample from a subject, and wherein the computer-readable medium includes instructions that, when executed by the processor, cause the computer to: analyze the data set to detect and quantify one or more somatic mutations at the locus; calculate a selection coefficient for the locus; and compare the selection coefficient calculated for the locus with a threshold value to detect positive, neutral, or negative selection at the locus.

In still some embodiments, the present invention is directed to a computer-implemented method for classifying a subject as having clonal hematopoiesis of indeterminate potential (CHIP), the method including: receiving a first data set in a computer having a processor and a computer-readable medium, wherein the first data set includes a plurality of sequence reads obtained by sequencing a plurality of cell-free nucleic acids in a test sample from a subject, and wherein the computer-readable medium includes instructions that, when executed by the processor, cause the computer to: analyze the plurality of sequence reads to detect and quantify one or more somatic mutations at a locus known to be associated with CHIP; and classify the subject as having CHIP when one or more somatic mutation are detected at the locus.

In still some embodiments, the present invention is directed to a computer-implemented method for assessing a disease state of a subject, the method including: receiving a first data set in a computer having a processor and a computer-readable medium, wherein the first data set includes a plurality of sequence reads obtained by sequencing a plurality of nucleic acids in a first test sample obtained from a subject at a first time point, and wherein the computer-readable medium includes instructions that, when executed by the processor, cause the computer to: analyze the plurality of sequence reads to detect and quantify one or more somatic mutations at the locus; calculate a first selection coefficient for the locus; receive a second data set in the computer, wherein the second data set includes a plurality of sequence reads obtained by sequencing a plurality of nucleic acids in a second test sample obtained from the subject at a second time point; analyze the plurality of sequence reads to detect and quantify one or more somatic mutations at the locus; calculate a second selection coefficient for the locus; and compare the first selection coefficient and the second selection coefficient, wherein an increase in the second selection coefficient relative to the first coefficient indicates progression in the disease state, and wherein a decrease in the second selection coefficient relative to the first selection coefficient indicates an improvement in the disease state.

Further, in some embodiments, a method for detecting positive, neutral, or negative selection at a locus includes: (a) obtaining a test sample from a subject, wherein the test sample includes a plurality of ribonucleic acid (RNA) molecules; (b) preparing a sequencing library from the plurality of RNA molecules; (c) sequencing the library to obtain a plurality of sequence reads, wherein the sequence reads are derived from the plurality of RNA molecules; (d) analyzing the plurality of sequence reads to detect and quantify one or more somatic mutations at the locus; (e) determining a selection coefficient for the locus; and (f) comparing the selection coefficient determined for the locus with a threshold value and detecting positive, neutral, or negative selection at the locus based on the comparison.

In some embodiments, a method for classifying a subject as having clonal hematopoiesis of indeterminate potential (CHIP) includes: (a) obtaining a test sample from a subject, wherein the test sample includes a plurality of ribonucleic acid (RNA) molecules; (b) preparing a sequencing library from the plurality of RNA molecules; (c) sequencing the library to obtain a plurality of sequence reads, wherein the sequence reads are derived from the plurality of RNA molecules; (d) analyzing the plurality of sequence reads to detect and quantify one or more somatic mutation at a locus known to be associated with CHIP; and (e) classifying the subject as having CHIP when the one or more somatic mutations at the locus are detected.

In some embodiments, a computer-implemented method for detecting positive, neutral, or negative selection at a locus includes: receiving a first data set in a computer having a processor and a computer-readable medium, wherein the first data set includes a plurality of sequence reads obtained by sequencing a plurality of ribonucleic acid (RNA) molecules in a test sample from a subject, and wherein the computer-readable medium includes instructions that, when executed by the processor, cause the computer to: analyze the data set to detect and quantify one or more somatic mutations at the locus; calculate a selection coefficient for the locus; and compare the selection coefficient calculated for the locus with a threshold value to detect positive, neutral, or negative selection at the locus.

In some embodiments, a computer-implemented method for classifying a subject as having clonal hematopoiesis of indeterminate potential (CHIP) includes: receiving a first data set in a computer having a processor and a computer-readable medium, wherein the first data set includes a plurality of sequence reads obtained by sequencing a plurality of ribonucleic acid (RNA) molecules in a test sample from a subject, and wherein the computer-readable medium includes instructions that, when executed by the processor, cause the computer to: analyze the plurality of sequence reads to detect and quantify one or more somatic mutations at a locus known to be associated with CHIP; and classify the subject as having CHIP when one or more somatic mutation are detected at the locus.

In some embodiments, a method for assessing a risk of developing a disease state, detecting a disease state, and/or diagnosing a disease state, comprises: detecting, from a cell-free nucleic acid sample obtained from a subject, a somatic mutation for at least one gene in a set of genes, wherein the set of genes comprises three or more of DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B.

In some aspects, the set of genes comprises five or more of DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B.

In some aspects, the set of genes comprises ten or more of DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B.

In some aspects, the set of genes consists of TET2, PPM1D, DNMT3A, ASXL1, and CHEK2.

In some aspects, the set of genes further includes one or more of TP53, ATM, SF3B1, JAK2, and KDR.

In some aspects, the set of genes consists of CHEK2, DNMT3A, SRSF2, TET2, and TP53.

In some aspects, the set of genes further includes one or more of CCND2, CBL, KRAS, MYD88, RAD21, and SF3B1.

In some aspects, the set of genes consists of ASKL1, CDKN1B, DNMT3A, PPM1D, and TET2.

In some aspects, the set of genes further includes one or more of CBL, CHEK2, EWSR1, RAD21, and SH2B3.

In some aspects, the cell-free nucleic acid sample is blood-based.

In some aspects, the method further comprises: detecting for the somatic mutation at a locus or loci associated with each gene in the set of genes; determining, based on the detected somatic mutation, a selection coefficient for the gene, wherein the selection coefficient is indicative of a functional impact level of the detected somatic mutation; and comparing the selection coefficient determined for the locus with a threshold value and detecting positive, neutral, or negative selection at the locus based on the comparison.

In some aspects, the selection coefficient is based on an allele frequency (AF) of the somatic mutation detected at the gene.

In some aspects, the method further comprises: detecting at least one missense mutation and at least one nonsense mutation at a locus or loci associated with a gene in the set of genes; and determining, based on the detection, that the gene is a tumor suppressor gene.

In some aspects, the method further comprises: detecting at least one missense mutation at a locus or loci associated with a gene in the set of genes; and determining, based on the detection, that the gene is an oncogene.

In some aspects, the method further comprises determining the positive selection based on the detection of the mutation at the gene, wherein the positive selection indicates presence of clonal hematopoiesis of indeterminate potential (CHIP) at the subject.

In some aspects, the method further comprises: developing a therapy, prognosis, or diagnosis in accordance with the gene and the type of mutation detected at the gene, wherein the type of mutation detected comprises at least one missense mutation

In some embodiments, a method of detecting clonal hematopoiesis of indeterminate potential (CHIP) in a subject, comprises: obtaining a cell-free nucleic acid (cfNA) sample from the subject; detecting, from the cfNA sample, a somatic mutation at one or more loci in a plurality of genes selected from three or more of DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B; and detecting CHIP in the subject when the somatic mutation is detected in each of the plurality of genes.

In some aspects, the plurality of genes comprises five or more of DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B.

In some aspects, the plurality of genes comprises ten or more of DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B.

In some aspects, the plurality of genes consists of TET2, PPM1D, DNMT3A, ASXL1, and CHEK2.

In some aspects, the set of genes further includes one or more of TP53, ATM, SF3B1, JAK2, and KDR.

In some aspects, the set of genes consists of CHEK2, DNMT3A, SRSF2, TET2, and TP53.

In some aspects, the set of genes further includes one or more of CCND2, CBL, KRAS, MYD88, RAD21, and SF3B1.

In some aspects, the set of genes consists of ASKL1, CDKN1B, DNMT3A, PPM1D, and TET2.

In some aspects, the set of genes further includes one or more of CBL, CHEK2, EWSR1, RAD21, and SH2B3.

In some aspects, wherein the somatic mutation comprises a missense mutation.

In some aspects, the somatic mutation comprises a nonsense mutation.

In some aspects, the method further comprises: determining a selection coefficient based on the detected somatic mutation, wherein the selection coefficient quantifies a strength of effect on CHIP of the detected somatic mutation; and determining, based on comparing the selection coefficient with a threshold value, the positive, neutral, or negative selection.

In some aspects, the strength of effect is based on whether the somatic mutation is a missense mutation at the gene or both a missense mutation and a nonsense mutation at the gene.

In some aspects, positive selection is determined when the detected somatic mutation is a missense mutation detected at the DNMT3A gene.

In some aspects, positive selection is determined when the detected somatic mutation is a nonsense mutation detected at the PPM1D gene.

### BRIEF DESCRIPTION OF THE DRAWINGS

FIG. 1 is a flowchart of a method for detecting positive, neutral, or negative selection at a locus, in accordance with various embodiments of the present invention.

FIG. 2 is a flowchart of a method for classifying a subject as having clonal hematopoiesis of indeterminate potential (CHIP), in accordance with various embodiments of the present invention.

FIG. 3 is a flowchart of a method for assessing a disease state of a subject, in accordance with various embodiments of the present invention.

FIG. 4 is a flowchart of a computer-implemented method for detecting positive, neutral, or negative selection at a locus, in accordance with various embodiments of the present invention.

FIG. 5 is a flowchart of a computer-implemented method for classifying a subject as having clonal hematopoiesis of indeterminate potential (CHIP), in accordance with various embodiments of the present invention.

FIG. 6 is a flowchart of a computer-implemented method for assessing a disease state of a subject, in accordance with various embodiments of the present invention.

FIG. 7 is a dot plot showing the number of WBC matched nonsynonymous mutations detected per subject versus their age, in accordance with various embodiments of the present invention.

FIG. 8 is a plot showing the distribution of WBC matched variant allele frequencies detected in subjects with cancer and healthy (e.g., non-cancer) subjects, in accordance with various embodiments of the present invention.

FIG. 9 is a plot showing the frequencies of WBC matched mutation detected in various genes correcting for gene length, in accordance with various embodiments of the present invention.

FIG. 10 is a plot showing the percentage of subjects where at least one WBC matched mutation was detected in various genes, in accordance with various embodiments of the present invention.

FIG. 11 is a table showing genes with evidence of selection (e.g., with a determined selection coefficient>1), in accordance with various embodiments of the present invention.

FIG. 12 is a block diagram of a processing system for processing sequence reads, in accordance with various embodiments of the present invention.

FIG. 13 is a flowchart of a method for determining variants of sequence reads, in accordance with various embodiments of the present invention.

FIG. 14 is a diagram of an application of a Bayesian hierarchical model, in accordance with various embodiments of the present invention.

FIG. 15A shows dependencies between parameters and sub-models of a Bayesian hierarchical model for determining true single nucleotide variants, in accordance with various embodiments of the present invention.

FIG. 15B shows dependencies between parameters and sub-models of a Bayesian hierarchical model for determining true insertions or deletions, in accordance with various embodiments of the present invention.

FIG. 16A illustrates a diagram associated with a Bayesian hierarchical model, in accordance with various embodiments of the present invention.

FIG. 16B illustrates another diagram associated with a Bayesian hierarchical model, in accordance with various embodiments of the present invention.

FIG. 17A is a diagram of determining parameters by fitting a Bayesian hierarchical model, in accordance with various embodiments of the present invention.

FIG. 17B is a diagram of using parameters from a Bayesian hierarchical model to determine a likelihood of a false positive, in accordance with various embodiments of the present invention.

FIG. 18 is a flowchart of a method for training a Bayesian hierarchical model, in accordance with various embodiments of the present invention.

FIG. 19 is a flowchart of a method for scoring candidate variants of a given nucleotide mutation, in accordance with various embodiments of the present invention.

FIG. 20 is a flowchart of a method for using a joint model to process cell free nucleic acid samples and genomic nucleic acid samples, in accordance with various embodiments of the present invention

FIG. 21 is a diagram of an application of a joint model, in accordance with various embodiments of the present invention.

FIG. 22 is a diagram of observed counts of variants in samples from healthy individuals, in accordance with various embodiments of the present invention.

FIG. 23 is a diagram of example parameters for a joint model, in accordance with various embodiments of the present invention.

FIG. 24 is a diagram of a set of genes detected from targeted sequencing assays using a joint model, in accordance with various embodiments of the present invention.

FIG. 25 is a diagram of another set of genes detected from targeted sequencing assays using a joint model, in accordance with various embodiments of the present invention.

FIG. 26 is a flowchart of a method for tuning a joint model to process cell free nucleic acid samples and genomic nucleic acid samples, in accordance with various embodiments of the present invention.

FIG. 27A is a table of example counts of candidate variants of cfDNA samples, in accordance with various embodiments of the present invention.

FIG. 27B is a table of example counts of candidate variants of cfDNA samples from healthy individuals, in accordance with various embodiments of the present invention.

FIG. 28 is a diagram of candidate variants plotted based on ratio of cfDNA and gDNA, in accordance with various embodiments of the present invention.

FIG. 29A depicts a process of generating an artifact distribution and a non-artifact distribution using training variants, in accordance with various embodiments of the present invention.

FIG. 29B depicts sequence reads that are categorized in an artifact training data category, in accordance with various embodiments of the present invention.

FIG. 29C depicts sequence reads that are categorized in the non-artifact training data category, in accordance with various embodiments of the present invention.

FIG. 29D depicts sequence reads that are categorized in the reference allele training data category, in accordance with various embodiments of the present invention.

FIG. 29E is an example depiction of a process for extracting a statistical distance from edge feature, in accordance with various embodiments of the present invention.

FIG. 29F is an example depiction of a process for extracting a significance score feature, in accordance with various embodiments of the present invention.

FIG. 29G is an example depiction of a process for extracting an allele fraction feature, in accordance with various embodiments of the present invention.

FIG. 29H depicts an example distribution used for identifying edge variants, in accordance with various embodiments of the present invention.

FIG. 29I depicts an example distribution used for identifying edge variants, in accordance with various embodiments of the present invention.

FIG. 30A depicts a block diagram flow process for determining a sample-specific predicted rate, in accordance with various embodiments of the present invention.

FIG. 30B depicts the application of an edge variant prediction model for identifying edge variants, in accordance with various embodiments of the present invention.

FIG. 31 depicts a flow process of identifying and reporting edge variants detected from a sample, in accordance with various embodiments of the present invention.

FIG. 32 is a flowchart of a method for processing candidate variants using different types of filters and models, in accordance with various embodiments of the present invention.

FIG. 33 is a plot showing a number of mutations by gene, in accordance with various embodiments of the present invention.

FIG. 34 is a plot showing a comparison of nonsense and missense selection coefficients for various genes, in accordance with various embodiments of the present invention.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF THE INVENTION

Reference will now be made in detail to several embodiments, examples of which are illustrated in the accompanying figures. It is noted that wherever practicable similar or like reference numbers may be used in the figures and may indicate similar or like functionality. For example, a letter after a reference numeral, such as “sequence reads 1180A,” indicates that the text refers specifically to the element having that particular reference numeral. A reference numeral in the text without a following letter, such as “sequence reads 1180,” refers to any or all of the elements in the figures bearing that reference numeral (e.g. “sequence reads 1180” in the text refers to reference numerals “sequence reads 1180A” and/or “sequence reads 1180B” in the figures).

### I. DEFINITIONS

The term “individual” refers to a human individual. The term “healthy individual” refers to an individual presumed to not have a cancer or disease. The term “subject” refers to an individual who is known to have, or potentially has, a cancer or disease.

The term “sequence reads” refers to nucleotide sequences read from a sample obtained from an individual. Sequence reads can be obtained through various methods known in the art.

The term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

The term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

The term “indel” refers to any insertion or deletion of one or more base pairs having a length and a position (which may also be referred to as an anchor position) in a sequence read. An insertion corresponds to a positive length, while a deletion corresponds to a negative length.

The term “mutation” refers to one or more SNVs or indels.

The term “true positive” refers to a mutation that indicates real biology, for example, presence of a potential cancer, disease, or germline mutation in an individual. True positives are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive. Generally, false positives may be more likely to occur when processing sequence reads associated with greater mean noise rates or greater uncertainty in noise rates.

The term “cell free nucleic acid,” “cell free DNA,” or “cfDNA” refers to nucleic acid fragments that circulate in an individual's body (e.g., bloodstream) and originate from one or more healthy cells and/or from one or more cancer cells.

The term “circulating tumor DNA” or “ctDNA” refers to nucleic acid fragments that originate from tumor cells or other types of cancer cells, which may be released into an individual's bloodstream as a result of biological processes such as apoptosis or necrosis of dying cells or actively released by viable tumor cells.

The term “genomic nucleic acid,” “genomic DNA,” or “gDNA” refers to nucleic acid including chromosomal DNA that originate from one or more healthy cells.

The term “alternative allele” or “ALT” refers to an allele having one or more mutations relative to a reference allele, e.g., corresponding to a known gene.

The term “sequencing depth” or “depth” refers to a total number of read segments from a sample obtained from an individual.

The term “alternate depth” or “AD” refers to a number of read segments in a sample that support an ALT, e.g., including mutations of the ALT.

The term “reference depth” refers to a number of read segments in a sample that includes a reference allele at a candidate variant location.

The term “alternate frequency” or “AF” refers to the frequency of a given ALT.

The AF may be determined by dividing the corresponding AD of a sample by the depth of the sample for the given ALT.

The term “variant” or “true variant” refers to a mutated nucleotide base at a position in the genome. Such a variant can lead to the development and/or progression of cancer in an individual.

The term “edge valiant” refers to a mutation located near an edge of a sequence read, for example, within a threshold distance of nucleotide bases from the edge of the sequence read.

The term “candidate variant,” “called variant,” “putative variant,” or refers to one or more detected nucleotide variants of a nucleotide sequence, for example, at a position in the genome that is determined to be mutated. Generally, a nucleotide base is deemed a called variant based on the presence of an alternative allele on sequence reads obtained from a sample, where the sequence reads each cross over the position in the genome. The source of a candidate variant may initially be unknown or uncertain. During processing, candidate variants may be associated with an expected source such as gDNA (e.g., blood-derived) or cells impacted by cancer (e.g., tumor-derived). Additionally, candidate variants may be called as true positives.

The term “non-edge variant” refers to a candidate variant that is not determined to be resulting from an artifact process, e.g., using an edge variant filtering method described herein. In some scenarios, a non-edge variant may not be a true variant (e.g., mutation in the genome) as the non-edge variant could arise due to a different reason as opposed to one or more artifact processes.

The term “clonal hematopoiesis” refers to clonal expansion of hematopoietic stem cells harboring one or more somatic mutations in common resulting in the formation of a genetically distinct subpopulation of blood cells.

The term “positive selection” refers to a somatic mutation or variant (e.g., a driver mutation) that confers a selective advantage or proliferation advantage on a cell line resulting in clonal expansion of the cell line. The term “negative selection” refers to a somatic mutation or variant that confers a selective disadvantage to the cell resulting in death or senescence. The term “neutral selection” refers to a somatic mutation or variant that does not provide a selective advantage or selective disadvantage on a cell line.

### II. DETECTING POSITIVE, NEGATIVE, OR NEUTRAL SELECTION IN CLONAL EXPANSION AND/OR CLONAL HEMATOPOIESIS

Cells accumulate somatic mutations throughout life. Under the pressures of selection, these mutations can be categorized as those that confer a selective disadvantage to the cell resulting in death or senescence (negative selection), those that are selectively neutral, and those that confer a selective advantage, increasing proliferation or survival (i.e. driver mutations, known as positive selection). Here we describe methods to identify signals of selection from somatic variants called in cfDNA derived from WBC. This enables the unbiased identification of genes under positive, neutral, or negative selection from a cohort of sequenced individuals. Genes under positive selection that are associated with a particular cohort may be candidates for therapy development, or, may have predictive power for prognostication and or diagnosis.

As described above, the present invention is directed to methods and systems for assessing one or more somatic variants in a biological sample. In various embodiments, the present invention includes detecting positive, neutral, or negative selection at a locus, classifying a subject as having clonal hematopoiesis of indeterminate potential (CHIP), and assessing a disease state of a subject. In some embodiments, selection at a locus, CHIP, or disease state assessment is based on white blood cell (WBC) matched cell-free DNA variants in a biological fluid sample, such as a blood sample.

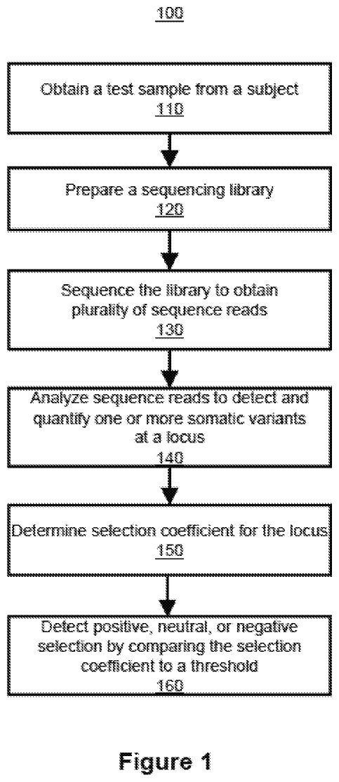

FIG. 1 is a flowchart illustrating a method 100 for detecting positive, neutral, or negative selection at a locus, in accordance with various embodiments of the present invention. Method 100 includes, but is not limited to, the following steps.

As shown in FIG. 1, at step 110 a test sample comprising a plurality of nucleic acids (e.g., a plurality of cell-free nucleic acids (cfNAs)) is obtained from a subject for testing. The subject may be a patient suspected of having, at risk of having, or known to have a disease state. For example, in some embodiments, a test sample is obtained from a patient suspected of having, at risk of having, or known to have cancer, cardiovascular disease, a neurodegenerative disease, or other disease.

In some examples, the test sample is a biological fluid sample selected from the group consisting of blood, plasma, serum, urine, saliva, fecal samples, and any combination thereof. In some examples, the test sample is a biological test sample including one or more cells (e.g., blood cells). Still, in some examples, the test sample or biological test sample comprises a test sample selected from the group consisting of whole blood, a blood fraction, a tissue biopsy, pleural fluid, pericardial fluid, cerebrospinal fluid, peritoneal fluid, and any combination thereof. In some examples, the sample is a plasma sample from a cancer patient, or a patient suspected of having cancer. In some examples, the test sample or biological test sample comprises a plurality of cell-free nucleic acids (e.g., cell-free DNA (cfDNA) and/or cell-free RNA (cfRNA)) fragments. In some examples, the nucleic acids are DNA or RNA molecules isolated from one or more cells (e.g., white blood cells (WBCs)). In some examples, the test sample or biological test sample comprises a plurality of cell-free nucleic acids (e.g., cell-free DNA and RNA) fragments originating from healthy cells and from unhealthy cells (e.g., cancer cells). Optionally, in some examples, cell-free nucleic acids (e.g., cfDNA and/or cfRNA) are extracted and/or purified from the test sample before proceeding with subsequent library preparation steps. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (Qiagen). In some examples, the sample is a fragmented genomic DNA (gDNA) sample (e.g., a sheared gDNA sample).

At step 120 a sequencing library is prepared from the plurality of nucleic acids (e.g., cfNAs). In general, a sequencing library may be prepared by any known means in the art. For example, adapters are ligated to the ends of the nucleic acid molecules obtained from step 110 to generate a plurality of adapter-fragment constructs. The ligation reaction can be performed using any suitable ligase enzyme which joins the adapters to the nucleic acid fragments to form adapter-fragment constructs. In one example, the nucleic acid fragments are double-strand DNA (dsDNA) fragments and a ligation reaction is performed using a DNA ligase (e.g., T4 or T7 DNA ligase) to ligate dsDNA adapters to the dsDNA fragments. In some examples, the ends of nucleic acid molecules (e.g., dsDNA molecules) are repaired using T4 DNA polymerase and/or Klenow polymerase and phosphorylated with a polynucleotide kinase enzyme prior to ligation of the adapters. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter. Ligation of single-strand adapters, or RNA adapters is also contemplated in the practice of the present invention. For example, in some cases ssRNA adapters are ligated to the ends of RNA fragments.

In some examples, the sequencing adapters can include a unique molecular identifier (UMI) sequence, such that, after library preparation, the sequencing library will include UMI tagged amplicons derived from dsDNA fragments. In one example, unique sequence tags (e.g., unique molecular identifiers (UMIs)) can be used to identify unique nucleic acid sequences from a test sample. For example, differing unique sequence tags (e.g., UMIs) can be used to differentiate various unique nucleic acid sequence fragments originating from the test sample. In another example, unique sequence tags (e.g., UMIs) can be used to reduce amplification bias, which is the asymmetric amplification of different targets due to differences in nucleic acid composition (e.g., high G/C content). The unique sequence tags (e.g., UMIs) can also be used to discriminate between nucleic acid mutations that arise during amplification. In one example, the unique sequence tag can comprise a short oligonucleotide sequence having a length of from about 2 nt to about 100 nt, from about 2 nt to about 60 nt, from about 2 to bout 40 nt, or from about 2 to bout 20 nt. In another example, the UMI tag may comprise a short oligonucleotide sequence greater than 5, 6, 7, 8, 9, 10, 11, 12, 13, 14, 15, 16, 17, or 18 nucleotides (nt) in length.

The unique sequence tags can be present in a multi-functional nucleic acid sequencing adapter, which sequencing adapter can comprise both a unique sequence tag and a universal priming site. In another example, the sequencing adapters utilized include a universal primer and/or one or more sequencing oligonucleotides for use in subsequent cluster generation and/or sequencing (e.g., known P5 and P7 sequences for used in sequencing by synthesis (SBS) (IIlumina, San Diego, Calif.)).

After adapter ligation, the adapter-fragment constructs are amplified to generate a sequencing library. For example, the adapter-modified dsDNA molecules can be amplified by PCR using a DNA polymerase and a reaction mixture containing primers.

At step 130 the library, or a portion thereof, is sequenced to obtain a plurality of sequence reads. In general, any method known in the art can be used to obtain sequence reads or sequencing data from the sequencing library. For example, sequencing data or sequence reads from the cell-free DNA sample can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some examples, sequencing is massively parallel sequencing using sequencing-by-synthesis with reversible dye terminators. In other examples, sequencing is sequencing-by-ligation. In yet other examples, the sequencing step is performed using single molecule sequencing. In still another example, sequencing is paired-end sequencing. Optionally, an amplification step is performed prior to sequencing.

In one example, the sequencing is whole-genome sequencing (WGS). In another example, the sequencing is a whole-genome bisulfite sequencing (WGBS). In still another example, the sequencing is whole-exome sequencing (WES). In still another example, the sequencing is RNA sequencing (RNA-seq), transcriptome sequencing or whole-transcriptome shotgun sequencing (WTSS). For RNA sequencing it is common to convert isolated RNA molecules to complementary DNA (cDNA) molecules using reverse transcriptase, prior to library preparation and sequencing. In some examples, the sequencing library is sequenced to a depth of at least 10×, at least 20×, at least 30×, at least sox, or at least 100×. In other examples, the sequencing library is sequenced to a depth of at least 500×, at least 1,000×, at least 2,000×, at least 3,000×, or at least 10,000×.

In yet another example, the sequencing comprises a targeted sequencing process. For example, the sequencing library can be enriched for one or more targeted nucleic acids prior to sequencing step 130. During enrichment, hybridization probes (also referred to herein as “probes”) are used to target, and pull down, nucleic acid fragments informative for the presence or absence of cancer (or disease), cancer status, or a cancer classification (e.g., cancer type or tissue of origin). For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand The probes can range in length from 10 s, 100 s, or 1000 s of base pairs. In one example, the probes are designed based on a gene panel to analyze particular mutations or target regions of the genome (e.g., of the human or another organism) that are suspected to correspond to certain cancers or other types of diseases. Moreover, the probes can cover overlapping portions of a target region. As one of ordinary skill in the art would appreciate, other means for enrichment of genes and nucleic acids derived from genes can be used.

In some examples, one or more (or all) of the probes are designed based on a gene panel to analyze particular mutations or target regions of the genome (e.g., of the human or another organism) that are suspected to correspond to certain cancers, cardiovascular diseases, or other types of diseases. By using a targeted gene panel rather than sequencing all expressed genes of a genome, also known as “whole exome sequencing,” the method 100 can be used to increase sequencing depth of the target regions, where depth refers to the count of the number of times a given target sequence within the sample has been sequenced. Increasing sequencing depth reduces required input amounts of the nucleic acid sample.

In some examples, the targeted enrichment step comprises an enrichment panel comprising from about 10 to bout 10,000 targeted genes, about 50 to bout 1,000 genes, or about 100 to bout 500 genes.

In some examples, the sequence reads are aligned to a reference genome using known methods in the art to determine alignment position information. The alignment position information can indicate a beginning position and an end position of a region in the reference genome that corresponds to a beginning nucleotide base and end nucleotide base of a given sequence read. Alignment position information can also include sequence read length, which can be determined from the beginning position and end position. A region in the reference genome can be associated with a gene or a segment of a gene.

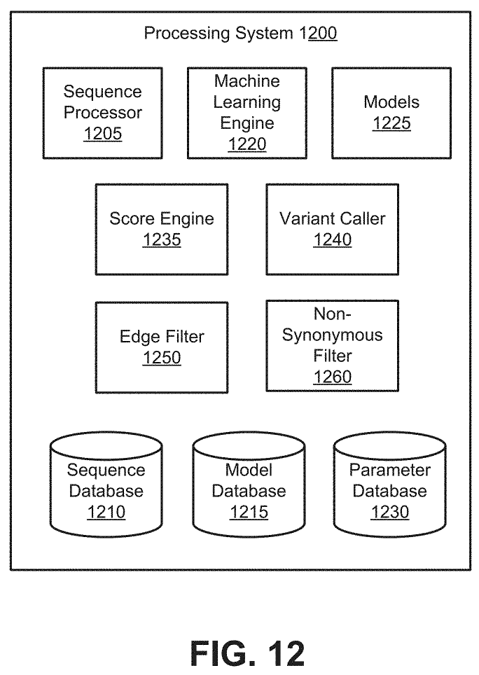

In various embodiments, a sequence read is comprised of a read pair denoted as R₁ and R₂. For example, the first read R₁ can be sequenced from a first end of a nucleic acid fragment whereas the second read R₂ can be sequenced from the second end of the nucleic acid fragment. Therefore, nucleotide base pairs of the first read R₁ and second read R₂ can be aligned consistently (e.g., in opposite orientations) with nucleotide bases of the reference genome. Alignment position information derived from the read pair R₁ and R₂ can include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as variant calling, as described below with respect to FIG. 12.

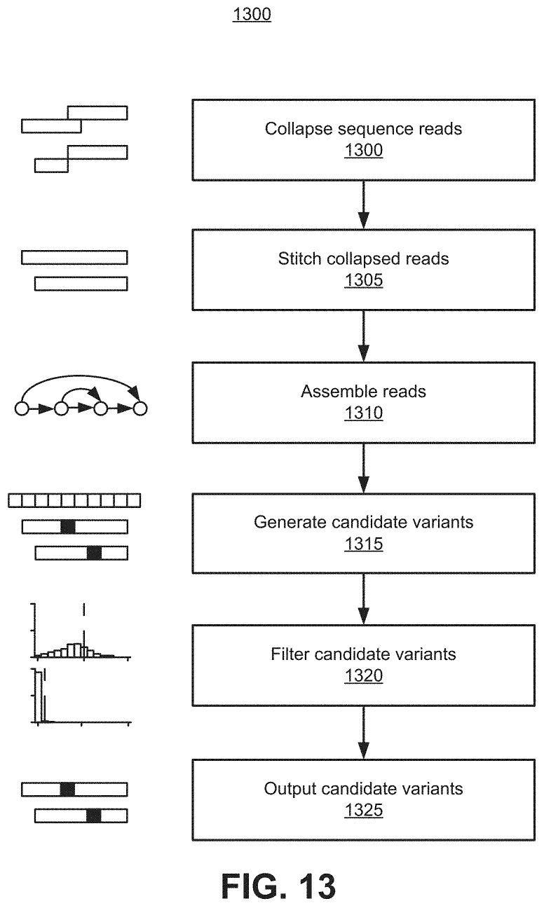

As shown in FIG. 1, at step 140, the plurality of sequence reads are analyzed to detect and quantify one or more somatic mutations (or variants) at a locus, or one or more somatic mutations (or variants) at a plurality of loci. Any known means in the art can be used to detect and quantify somatic mutations from the sequencing reads or sequencing data. For example, a variant calling pipeline can be used to detect and quantify somatic mutations or variants, as described in further detail below in conjunction with FIG. 13.

In some examples, analyzing the plurality of sequence reads to detect and quantify the one or more somatic mutations apply a noise model, as described in further detail hereinbelow. In still some examples, analyzing the plurality of sequence reads applies a read mis-mapping model to identify and account for artifactual somatic mutations (or variant) calls that arise from mis-mapping. In one example, as described elsewhere herein, one or more white blood cell (WBC) matched cell-free DNA mutations (or variants) can be detected or identified in accordance with the present invention. For example, one or more somatic mutations are detected from joint modeling or mixture modeling both the cell-free nucleic acid somatic mutation and the one or more white blood cell derived somatic mutations.

In some examples, sequence reads covering one or more loci or genes known to be associated with a disease state can be analyzed to detect or identify a somatic mutation at that loci or genes. For example, one or more loci or genes known to be, or suspected of being, associated with cancer can be analyzed for somatic mutations (or variants) at those loci or genes. In another example, one or more loci or genes known to be associated, or suspected of being associated, with clonal hematopoiesis of indeterminate potential (CHIP) are analyzed. For example, DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B, or any combination thereof. In some examples, one or more loci or genes known to be, or suspected of being, associated with a neurodegenerative disease may be analyzed. In some examples, sequence reads can be analyzed for identification of a known somatic mutation in a subject (e.g., a known somatic mutation associated with a disease or disease state) to assess or infer how a subject will respond to a therapeutic treatment targeting that somatic mutation. In still some examples, sequence reads can be analyzed to identify previously unknown, or previously undetected somatic mutations (or variants) as potential targets for development of a therapeutic agent to treat a particular disease or disease state.

In one example, the one or more somatic mutations comprise one or more single-nucleotide variants. In other examples, the one or more somatic mutations comprise one or more small insertions and/or deletions (Indels). For example, the one or more somatic mutations comprise one or more nonsynonymous mutations, one or more missense mutations, one or more nonsense mutations, one or more truncating mutations, or one or more essential splice site mutations. In another example, the one or more somatic mutations are detected in one or more oncogenes or one or more tumor suppressor genes.

At step 150, a selection coefficient is determined or calculated for each of the one or more loci where a somatic mutation (or variant) is detected. In some examples, the selection coefficient is an estimate that directly enumerates the excess or deficit of mutations in a given loci, gene, group of genes, or even the whole exome (all genes) compared to the expectation for the background mutational process. For example, dN/dS (also referred to as Ka/Ks), as denoted by ω, can be used to estimate the excess or deficit of mutations in a given locus, gene, group of genes, or even the whole exome (all genes) compared to the expectation for the background mutational process. As used herein, dN/dS (e.g., see Minyata and Yasunaga, J. Mol. Evol. (1980)) is the ratio between the rate of nonsynonymous per nonsynonymous site and synonymous substitutions per synonymous site. In some examples with somatic mutations (or variants), the rate of mutations per site is used.

Poisson model: Mutations are distinguished by their mutation type and functional impact (e.g., nonsense (n), missense (m), essential splice site (e), and synonymous nonsynonymous mutation categories can be aggregated). Rates are measured per mutation site (e.g., C>T) and context (e.g., triplet context may be CCG). For simplicity, assuming a mutation type X, the synonymous rate is then modeled as a Poisson process:

n(x, s)=Pois(λ=d*r(x)*L(x, s))

where d is the density of substitutions per site, r(x) is the relative rate of mutations of type X per site, and L(x, s) is the number of wild type context sites in which an X change is synonymous. For nonsynonymous sites, an extra parameter ω represents the effect of selection on accumulation of mutations:

n(x, n)=Pois(λ=d*r(x)*L(x, n)*ω(n))

where n here represents the nonsense category of nonsynonymous mutations. The parameters ω are the dN/dS ratios inferred by the model after correcting for the rates of different substitution classes and for sequencing composition. Poisson regression can be used to obtain maximum likelihood estimates for all of the parameters above.

The above model can be extended to model variable mutations across loci (l) as a Gamma distribution. Because the negative binomial distribution is a Gamma-Poisson compound distribution, a negative binomial regression framework can be applied:

n(s, l)=Pois(λ)

λ=Gamma(α, β)

Covariates can then be used in this framework to improve the estimated background rate for a locus and reduce the unexplained variation in mutation rate. This leads to a reduction of dispersion of the underlying Gamma distribution, hence leading to more sensitivity for the detection of selection.

A likelihood ratio test can then be applied to inter selection, similar to traditional likelihood models used in phylogenetics. The percent of variants under selection at a locus k, f(k) can then be calculated as a deviation from neutrality from the value of w estimated at k,

f(k)=(ω−1)/ω

In other examples, dN/dS estimation for one or more somatic mutations can be modified to guard against confounding (see, e.g., Martincorena et al., Cell (2017)). For example, variation in mutation rate along the human genome can be taken into account. In another example, sequence context dependent mutational processes can be taken into account.

In one example, a selection coefficient is determined or calculated for one or more nonsynonymous mutations, one or more missense mutations, one or more nonsense mutations, one or more truncating mutations, and/or one or more essential splice site mutations. In another example, a selection coefficient is determined or calculated for one or more somatic mutations detected in one or more oncogenes and/or one or more tumor suppressor genes.

Finally, in step 160, the selection coefficient determined in step 150 for a locus, or for one or more loci, is compared with a threshold value for detection of positive, neutral, or negative selection at the locus, or one or more loci. In one example, positive selection is detected when the threshold value is greater than 1 (i.e., when the selection coefficient is greater than 1) with a q-value less than 0.05. In another example, negative selection is detected when the threshold value is less than 1 (i.e., when the selection coefficient is less than 1) with a q-value less than 0.05. In still another example, neutral selection is detected when the selection coefficient is about 1.

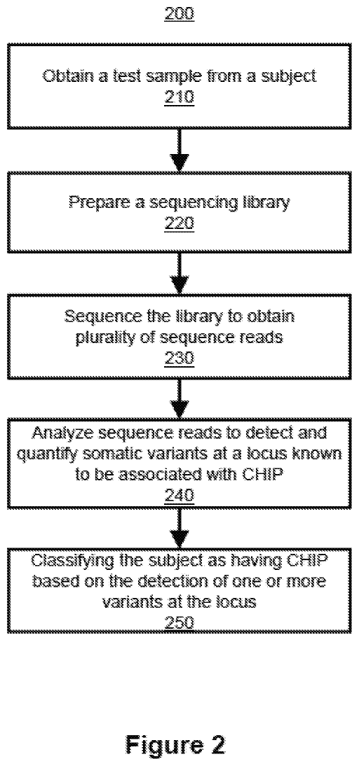

FIG. 2 a flowchart illustrating a method 200 for classifying a subject as having clonal hematopoiesis of indeterminate potential (CHIP), in accordance with some embodiments of the present invention. Method 200 includes, but is not limited to, the following steps.

As shown in FIG. 2, a test sample is obtained from a subject, wherein the test sample comprises a plurality of nucleic acids (e.g., a plurality of cell-free nucleic acids (cfNAs)). As described above, the subject may be a patient suspected of having, at risk of having, or known to have a disease state. For example, a test sample is obtained from a patient suspected of having, suspected of having, or known to have cancer, cardiovascular disease, a neurodegenerative disease, or other disease.

In some examples, the test sample is a biological fluid sample selected from the group consisting of blood, plasma, serum, urine, saliva, fecal samples, and any combination thereof. In another example, the test sample is a biological test sample including one or more cells (e.g., blood cells). In still another example, the test sample or biological test sample comprises a test sample selected from the group consisting of whole blood, a blood fraction, a tissue biopsy, pleural fluid, pericardial fluid, cerebrospinal fluid, peritoneal fluid, and any combination thereof. In other examples, the sample is a plasma sample from a cancer patient, or a patient suspected of having cancer. In accordance with some examples of the present invention, the test sample or biological test sample comprises a plurality of cell-free nucleic acids (e.g., cell-free DNA (cfDNA) and/or cell-free RNA (cfRNA)) fragments. In some examples, the nucleic acids are DNA or RNA molecules isolated from one or more cells (e.g., white blood cells (WBCs)). In other examples, the test sample or biological test sample comprises a plurality of cell-free nucleic acid (e.g., cell-free DNA and RNA) fragments originating from healthy cells and from unhealthy cells (e.g., cancer cells). Optionally, in one example, cell-free nucleic acids (e.g., cfDNA and/or cfRNA) can be extracted and/or purified from the test sample before proceeding with subsequent library preparation steps. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (Qiagen). In some examples, the sample can be, for example, a fragmented genomic DNA (gDNA) sample (e.g., a sheared gDNA sample).

At step 220 a sequencing library is prepared from the plurality of nucleic acids (e.g., cfNAs). In general, a sequencing library can be prepared by any known means in the art. For example, as described above in conjunction with FIG. 1, adapters can be ligated to the ends of the nucleic acid molecules obtained from step 210 to generate a plurality of adapter-fragment constructs. The ligation reaction can be performed using any suitable ligase enzyme known in the art. The sequencing adapters can include a unique molecular identifier (UMI) sequence, such that, after library preparation, the sequencing library will include UMI tagged amplicons derived from nucleic acid fragments, as described above. The unique sequence tags can be present in a multi-functional nucleic acid sequencing adapter, which sequencing adapter can comprise both a unique sequence tag and a universal priming site. In another example, as described above, the sequencing adapters utilized include a universal primer and/or one or more sequencing oligonucleotides for use in subsequent cluster generation and/or sequencing (e.g., known P5 and P7 sequences for used in sequencing by synthesis (SBS) (alumina, San Diego, Calif.)). After adapter ligation, the adapter-fragment constructs are amplified to generate a sequencing library. For example, the adapter-modified dsDNA molecules can be amplified by PCR using a DNA polymerase and a reaction mixture containing primers.

At step 230 the library, or a portion thereof, is sequenced to obtain a plurality of sequence reads. In general, any method known in the art can be used to obtain sequence reads or sequencing data from the sequencing library. For example, sequencing data or sequence reads from the cell-free DNA sample can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some examples, sequencing is massively parallel sequencing using sequencing-by-synthesis with reversible dye terminators. In other examples, sequencing is sequencing-by-ligation. In yet other examples, the sequencing step is performed using single molecule sequencing. In still another example, sequencing is paired-end sequencing. Optionally, an amplification step is performed prior to sequencing.

As described above in conjunction with FIG. 1, sequencing can be carried out using whole-genome sequencing (WGS), whole-genome bisulfite sequencing (WGBS), whole-exome sequencing (WES), or targeted sequencing. As described with respect to FIG. 1, in a targeted sequencing process, the sequencing library can be enriched for one or more targeted nucleic acids prior to sequencing step 230. The targeted enrichment step can utilize an enrichment panel comprising from about 10 to bout 10,000 targeted genes, about 50 to about 1,000 genes, or about 100 to bout 500 genes. In another example, the sequencing is RNA sequencing (RNA-seq), transcriptome sequencing, or whole-transcriptome shotgun sequencing (WTSS). Also as previously described, the sequencing library can be sequenced to a depth of at least 10×, at least 20×, at least 30×, at least 50×, at least 100×, at least 500×, at least 1,000×, at least 2,000×, at least 3,000×, or at least 10,000×.

In some embodiments, the sequence reads can be aligned to a reference genome using known methods in the art to determine alignment position information. The alignment position information can indicate a beginning position and an end position of a region in the reference genome that corresponds to a beginning nucleotide base and end nucleotide base of a given sequence read. Alignment position information can also include sequence read length, which can be determined from the beginning position and end position. A region in the reference genome can be associated with a gene or a segment of a gene.

In various embodiments, a sequence read is comprised of a read pair denoted as R₁ and R₂. For example, the first read R₁ is sequenced from a first end of a nucleic acid fragment whereas the second read R₂ is sequenced from the second end of the nucleic acid fragment. Therefore, nucleotide base pairs of the first read R₁ and second read R₂ can be aligned consistently (e.g., in opposite orientations) with nucleotide bases of the reference genome. Alignment position information derived from the read pair R₁ and R₂ can include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format can be generated and output for further analysis such as variant calling, as described below with respect to FIG. 12.

As shown in FIG. 2, at step 240, the plurality of sequence reads are analyzed to detect and quantify one or more somatic mutation at a locus known to be associated with or suspected of being associated with clonal hematopoiesis of indeterminate potential (CHIP). For example, DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B, or any combination thereof. Any known means in the art can be used to detect and quantify somatic mutations from the sequencing reads or sequencing data. For example, a variant calling pipeline can be used to detect and quantify somatic mutations or variants, as described in further detail below in conjunction with FIG. 13. In another example, sequence reads can be analyzed for identification of a known somatic mutation in a subject (e.g., a known somatic mutation associated with a disease or disease state) to assess or infer how a subject will respond to a therapeutic treatment targeting that somatic mutation. In still another example, sequence reads can be analyzed to identify previously unknown, or previously undetected somatic mutations (or variants) as potential targets for development of a therapeutic agent to treat a particular disease or disease state.

In one example, the one or more somatic mutations comprise one or more single-nucleotide variants. In another example, the one or more somatic mutations comprise one or more small insertions and/or deletions (Indels). For example, the one or more somatic mutations comprise one or more nonsynonymous mutations, one or more missense mutations, one or more nonsense mutations, one or more truncating mutations, or one or more essential splice site mutations. In another example, the one or more somatic mutations are detected in one or more oncogenes or one or more tumor suppressor genes.

In some examples, analyzing the plurality of sequence reads to detect and quantify the one or more somatic mutations apply a noise model, as described in further detail hereinbelow. In some examples, analyzing the plurality of sequence reads applies a read mis-mapping model to identify and account for artifactual somatic mutations (or variant) calls that may arise from mis-mapping. In one example, as described elsewhere herein, one or more white blood cell (WBC) matched cell-free DNA mutations (or variants) can be detected or identified in accordance with the present invention. For example, one or more somatic mutations are detected from joint modeling or mixture modeling both the cell-free nucleic acid somatic mutation and the one or more white blood cell derived somatic mutations.

Optionally, a selection coefficient is determined or calculated for each of the one or more loci where a somatic mutation (or variant) is detected. As previously described in conjunction with FIG. 1, the selection coefficient can be an estimate that directly enumerates the excess or deficit of mutations in a given loci, gene, group of genes, or even the whole exome (all genes) compared to the expectation for the background mutational process. For example, dN/dS (also referred to as Ka/Ks), as denoted by w, can be used to estimate the excess or deficit of mutations in a given loci, gene, group of genes, or even the whole exome (all genes) compared to the expectation for the background mutational process. As used herein, dN/dS (e.g., see Minyata and Yasunaga, J. Mol. Evol. (1980)) is the ratio between the rate of nonsynonymous per nonsynonymous site and synonymous substitutions per synonymous site. For somatic mutations (or variants), the rate of mutations per site can be analyzed.

As previously described, dN/dS estimation for one or more somatic mutations can be modified to guard against confounding (see, e.g., Martincorena et al., Cell (2017)). For example, variation in mutation rate along the human genome can be taken into account. In another example, sequence context dependent mutational processes can be taken into account.

In one example, a selection coefficient is determined or calculated for one or more nonsynonymous mutations, one or more missense mutations, one or more nonsense mutations, one or more truncating mutations, and/or one or more essential splice site mutations. In another example, a selection coefficient can be determined or calculated for one or more somatic mutations detected in one or more oncogenes and/or one or more tumor suppressor genes.

As step 250, the subject is classified as having CHIP when one or more somatic mutations are detected at one or more loci known to be associated with or suspected of being associated with CHIP.

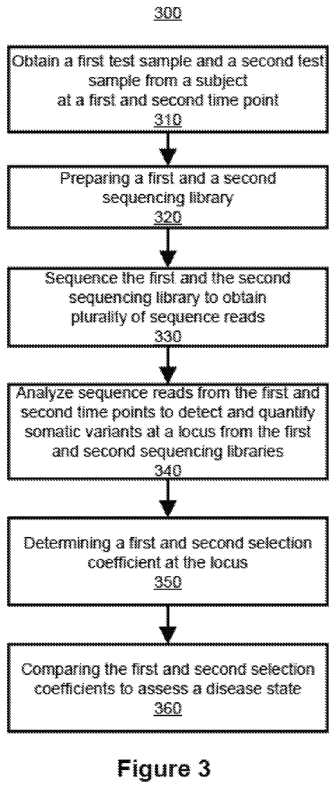

FIG. 3 is a flowchart illustrating a method 300 for assessing a disease state of a subject, in accordance with some embodiments of the present invention. Method 300 includes, but is not limited to, the following steps.

As shown in FIG. 3, at step 310, a first test sample and a second test sample comprising a plurality of nucleic acids are obtained from a subject at a first time point and a second time point, respectively. As described above, the subject can be a patient suspected of having, at risk of having, or known to have a disease state. For example, a test sample may be obtained from a patient suspected of having, suspected of having, or known to have cancer, cardiovascular disease, a neurodegenerative disease, or other disease.

In some examples, the test sample is a biological fluid sample selected from the group consisting of blood, plasma, serum, urine, saliva, fecal samples, and any combination thereof. In another example, the test sample is a biological test sample including one or more cells (e.g., blood cells). In still another example, the test sample or biological test sample comprises a test sample selected from the group consisting of whole blood, a blood fraction, a tissue biopsy, pleural fluid, pericardial fluid, cerebrospinal fluid, peritoneal fluid, and any combination thereof. In other examples, the sample is a plasma sample from a cancer patient, or a patient suspected of having cancer. In accordance with some embodiments, the first and second test sample are blood samples, and the plurality of nucleic acids are genomic DNA (gDNA) molecules derived from blood cells (e.g., white blood cells (WBCs)). In other examples, the first and second test sample or biological test sample comprises a plurality of cell-free nucleic acids (e.g., cell-free DNA (cfDNA) and/or cell-free RNA (cfRNA)) fragments. In some embodiments, the first and second test sample or biological test sample comprises a plurality nucleic acids are DNA or RNA molecules isolated from one or more cells (e.g., white blood cells (WRCs)). In some embodiments, the test samples or biological test samples comprises a plurality of cell-free nucleic acids (e.g., cell-free DNA and RNA) fragments originating from healthy cells and from unhealthy cells (e.g., cancer cells). Optionally, in one example, cell-free nucleic acids (e.g., cfDNA and/or cfRNA) can be extracted and/or purified from the test sample before proceeding with subsequent library preparation steps. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (Qiagen). In some embodiments, the sample can be, for example, a fragmented genomic DNA (gDNA) sample (e.g., a sheared gDNA sample).

In general, any time points could be used for collection of the first and second test samples and subsequent analysis. For example, the time points can be at least about 1 day apart, 2 days apart, 3 days apart, 4 days apart, 5 days apart, 6 days apart, 1 week apart, 1 month apart, 2 months apart, 3 months apart, 6 months apart, 7 months apart, 8 months apart, 9 months apart, 10 months apart, 11 months apart, or about 12 months apart. In another example, the time points can be semi-annually or annually, for example, every six months, one a year, once every other year, or once every two years. In still another example, the time points may be 1 year apart, 2 years apart, 3 years apart, 4 years apart, 5 years apart, 6 years apart, 7 years apart, 8 years apart, 9 years apart, or 10 years apart.

In some examples, the method 300 includes collecting a plurality of biological samples at a plurality of time points and detecting and quantifying one or more features from each of the plurality of biological test samples obtained at the plurality of time points for monitoring the progression of a disease condition and/or the regression of a disease condition (e.g., in response to a treatment regimen and/or a therapeutic treatment for a disease condition). For example, 2 or more, 3 or more, 4 or more, 5 or more, 10 or more, 15 or more, 20 or more, or 25 or more test samples can be obtained from a subject to monitor progression of a disease, regression of a disease, or to monitor response to a therapy to treat a disease.

At step 320, a first sequencing library and a second sequencing library are prepared from the plurality of nucleic acids obtained from the first test sample and the second test sample, respectively. In general, any known means in the art can be used to prepare the first and second sequencing libraries. For example, as described above in conjunction with FIG. 1, adapters can be ligated to the ends of the nucleic acid molecules obtained from step 210 to generate a plurality of adapter-fragment constructs. The ligation reaction can be performed using any suitable ligase enzyme known in the art. The sequencing adapters can include a unique molecular identifier (UMI) sequence, such that, after library preparation, the sequencing library will include UMI tagged amplicons derived from nucleic acid fragments, as described above. The unique sequence tags can be present in a multi-functional nucleic acid sequencing adapter, which sequencing adapter can comprise both a unique sequence tag and a universal priming site. In another example, as described above, the sequencing adapters utilized may include a universal primer and/or one or more sequencing oligonucleotides for use in subsequent cluster generation and/or sequencing (e.g., known P5 and P7 sequences for used in sequencing by synthesis (SBS) (Illumina, San Diego, Calif.)). After adapter ligation, the adapter-fragment constructs are amplified to generate a sequencing library. For example, the adapter-modified dsDNA molecules can be amplified by PCR using a DNA polymerase and a reaction mixture containing primers.

At step 330, the first and the second sequencing libraries, or portions thereof, are sequenced to obtain a plurality of sequence reads at one or more loci from the first sequencing library and a plurality of sequence reads at one or more loci from the second sequencing library, respectively. In general, any method known in the art can be used to obtain sequence reads or sequencing data from the first and second sequencing libraries. For example, sequencing data or sequence reads from the cell-free DNA sample can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some embodiments, sequencing is massively parallel sequencing using sequencing-by-synthesis with reversible dye terminators. In some embodiments, sequencing is sequencing-by-ligation. In some examples, the sequencing step is performed using single molecule sequencing. In still another example, sequencing is paired-end sequencing. Optionally, an amplification step is performed prior to sequencing.

As described above in conjunction with FIG. 1, the sequencing can be carried out using whole-genome sequencing (WGS), whole-genome bisulfite sequencing (WGBS), whole-exome sequencing (WES), or targeted sequencing. As described with respect to FIG. 1, in a targeted sequencing process, the sequencing library can be enriched for one or more targeted nucleic acids prior to sequencing step 230. The targeted enrichment step can utilize an enrichment panel comprising from about 10 to bout 10,000 targeted genes, about 50 to about 1,000 genes, or about 100 to bout 500 genes. In another example, the sequencing is RNA sequencing (RNA-seq), transcriptome sequencing, or whole-transcriptome shotgun sequencing (WTSS). Also as previously described, the sequencing library can be sequenced to a depth of at least 10×, at least 20×, at least 30×, at least 50×, at least 100×, at least 500×, at least 1,000×, at least 2,000×, at least 3,000×, or at least 10,000×.

In some embodiments, the sequence reads can be aligned to a reference genome using known methods in the art to determine alignment position information. The alignment position information can indicate a beginning position and an end position of a region in the reference genome that corresponds to a beginning nucleotide base and end nucleotide base of a given sequence read. Alignment position information can also include sequence read length, which can be determined from the beginning position and end position. A region in the reference genome can be associated with a gene or a segment of a gene.

In various embodiments, a sequence read is comprised of a read pair denoted as R₁ and R₂. For example, the first read R₁ can be sequenced from a first end of a nucleic acid fragment whereas the second read R₂ can be sequenced from the second end of the nucleic acid fragment. Therefore, nucleotide base pairs of the first read R₁ and second read R₂ can be aligned consistently (e.g., in opposite orientations) with nucleotide bases of the reference genome. Alignment position information derived from the read pair R₁ and R₂ can include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format can be generated and output for further analysis such as variant calling, as described below with respect to FIG. 12.

As shown in FIG. 3, at step 340, the plurality of sequence reads obtained from the first sequencing library and the plurality of sequence reads obtained from the second sequencing library, respectively, are analyzed to detect and quantify one or more somatic mutations from the first sequencing library at one or more loci and one or more somatic mutations from the second sequencing library at one or more loci, respectively. Any known means in the art can be used to detect and quantify somatic mutations from the sequencing reads or sequencing data. For example, a variant calling pipeline can be used to detect and quantify somatic mutations or variants, as described in further detail below in conjunction with FIG. 13.

In some embodiments, analyzing the plurality of sequence reads to detect and quantify the one or more somatic mutations can apply a noise model, as described in further detail hereinbelow. In still some embodiments, analyzing the plurality of sequence reads can further apply a read mis-mapping model to identify and account for artifactual somatic mutations (or variant) calls that may arise from mis-mapping. In one example, as described elsewhere herein, one or more white blood cell (WBC) matched cell-free DNA mutations (or variants) can be detected or identified in accordance with the present invention. For example, one or more somatic mutations are detected from joint modeling, or from mixture modeling both the cell-free nucleic acid somatic mutation and the one or more white blood cell derived somatic mutations.

In one example, sequence reads covering one or more loci or genes known to be associated with a disease state can be analyzed to detect or identify a somatic mutation at that loci or genes. For example, one or more loci or genes known to be, or suspected of being, associated with cancer can be analyzed for somatic mutations (or variants) at those loci or genes. In another embodiment, one or more loci or genes known to be associated, or suspected of being associated, with clonal hematopoiesis of indeterminate potential (CHIP) can be analyzed. For example, DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B, or any combination thereof. In still some embodiments, one or more loci or genes known to be, or suspected of being, associated with a neurodegenerative disease can be analyzed. In another example, sequence reads can be analyzed for identification of a known somatic mutation in a subject (e.g., a known somatic mutation associated with a disease or disease state) to assess or infer how a subject will respond to a therapeutic treatment targeting that somatic mutation. In still another example, sequence reads can be analyzed to identify previously unknown, or previously undetected somatic mutations (or variants) as potential targets for development of a therapeutic agent to treat a particular disease or disease state.

In one example, the one or more somatic mutations comprise one or more single-nucleotide variants. In other examples, the one or more somatic mutations comprise one or more small insertions and/or deletions (Indels). For example, the one or more somatic mutations comprise one or more nonsynonymous mutations, one or more missense mutations, one or more nonsense mutations, one or more truncating mutations, or one or more essential splice site mutations. In another example, the one or more somatic mutations are detected in one or more oncogenes or one or more tumor suppressor genes.

At step 350, a selection coefficient is determined or calculated for each of the one or more loci where a somatic mutation (or variant) is detected from the first sequencing library and from the second sequencing library, respectively. In accordance with some embodiments of the present invention, the first and second selection coefficients are estimates that directly enumerate the excess or deficit of mutations in a given loci, gene, group of genes, or even the whole exome (all genes) compared to the expectation for the background mutational process. For example, dN/dS (also referred to as Ka/Ks), as denoted by e, can be used to estimate the excess or deficit of mutations in a given loci, gene, group of genes, or even the whole exome tall genes) compared to the expectation for the background mutational process. As used herein, dN/dS (e.g., see Minyata and Yasunaga, 1980) is the ratio between the rate of nonsynonymous per nonsynonymous site and synonymous substitutions per synonymous site. For somatic mutations (or variants), the rate of mutations per site can be used.

As previously described, dN/dS estimation for one or more somatic mutations can be modified to guard against confounding (see, e.g., Martincorena et al., Cell (2017)). For example, variation in mutation rate along the human genome can be taken into account. In another example, sequence context dependent mutational processes can be taken into account.

In some embodiments, a selection coefficient can be determined or calculated for one or more nonsynonymous mutations, one or more missense mutations, one or more nonsense mutations, one or more truncating mutations, and/or one or more essential splice site mutations. In some embodiments, a selection coefficient can be determined or calculated for one or more somatic mutations detected in one or more oncogenes and/or one or more tumor suppressor genes.

Finally, at step 360, the first selection coefficient and the second selection coefficient are compared. In accordance with this embodiment, an increase in the second selection coefficient relative to the first coefficient indicates progression in the disease state, and a decrease in the second selection coefficient relative to the first selection coefficient indicates an improvement in the disease state.

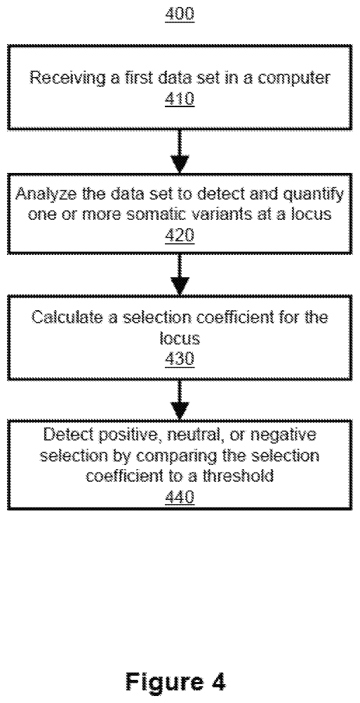

FIG. 4 is a flowchart illustrating an example computer-implemented method 400 for detecting positive, neutral, or negative selection at a locus. Computer-implemented method 400 includes, but is not limited to, the following steps.

As shown at step 410, a first data set is received in a computer comprising a processor and a computer-readable medium, wherein the first data set comprises a plurality of sequence reads obtained by sequencing a plurality of cell-free nucleic acids in a test sample from a subject. In accordance with some embodiments, the computer-readable medium comprises instructions that, when executed by the processor, cause the computer carry out one or more data analysis steps. In some embodiments, the first data set is obtained from sequencing the test sample using whole-genome sequencing (WGS), whole-genome bisulfite sequencing (WGBS), whole-exome sequencing (WES), or from a targeted sequencing process. In some embodiments, the data can be Obtained from a targeted sequencing process where the sequencing library has been enriched for one or more targeted nucleic acids prior to sequencing (as described above in conjunction with FIG. 1). The targeted enrichment step can utilize an enrichment panel comprising from about 10 to bout 10,000 targeted genes, about 50 to bout 1,000 genes, or about 100 to bout 500 genes. In another example, the first data set is obtained from sequencing the test sample using RNA sequencing (RNA-seq), transcriptome sequencing or whole-transcriptome shotgun sequencing (WTSS). In some embodiments, the sequencing data set is obtained from sequencing a sequencing library to a depth of at least 10×, at least 20×, at least 30×, at least 50×, or at least 100×. Further, in some embodiments, the sequencing library is sequenced to a depth of at least 500×, at least 1,000×, at least 2,000×, at least 3,000×, or at least 10,000×.

At step 420, the computer-readable medium comprises instructions that, when executed by the processor, cause the computer to analyze the data set to detect and quantify one or more somatic mutations at one or more loci. Any known means in the art can be used to detect and quantify somatic mutations from the sequencing reads or sequencing data. For example, a variant calling pipeline can be used to detect and quantify somatic mutations or variants, as described in further detail below in conjunction with FIG. 13.

As described above in conjunction with FIG. 1, the one or more somatic mutations can comprise one or more single-nucleotide variants. In some embodiments, the one or more somatic mutations comprise one or more small insertions and/or deletions (Indels). For example, the one or more somatic mutations comprise one or more nonsynonymous mutations, one or more missense mutations, one or more nonsense mutations, one or more truncating mutations, or one or more essential splice site mutations. In another example, the one or more somatic mutations can be detected in one or more oncogenes or one or more tumor suppressor genes.

As previously described, sequence reads can be analyzed for identification of a known somatic mutation in a subject (e.g., a known somatic mutation associated with a disease or disease state) to assess or infer how a subject will respond to a therapeutic treatment targeting that somatic mutation. In some embodiments, sequence reads can be analyzed to identify previously unknown, or previously undetected somatic mutations (or variants) as potential targets for development of a therapeutic agent to treat a particular disease or disease state.

In step 430, the computer-readable medium comprises instructions that, when executed by the processor, cause the computer to calculate a selection coefficient for each of the one or more loci where a somatic mutation (or variant) is detected. In accordance with some embodiments of the present invention, the selection coefficient is an estimate that directly enumerates the excess or deficit of mutations in a given loci, gene, group of genes, or even the whole exome (all genes) compared to the expectation for the background mutational process. For example, dN/dS (also referred to as Ka/Ks), as denoted by w, can be used to estimate the excess or deficit of mutations in a given loci, gene, group of genes, or even the whole exome (all genes) compared to the expectation for the background mutational process. As used herein, dN/dS (e.g., see Minyata and Yasunaga, 1980) is the ratio between the rate of nonsynonymous per nonsynonymous site and synonymous substitutions per synonymous site. For somatic mutations (or variants), the rate of mutations per site can be used.

As previously described, dN/dS estimation for one or more somatic mutations can be modified to guard against confounding (see, e.g., Martincorena et al., Cell (2017)). For example, variation in mutation rate along the human genome can be taken into account. In another example, sequence context dependent mutational processes can be taken into account.

In some embodiments, a selection coefficient can be determined or calculated for one or more nonsynonymous mutations, one or more missense mutations, one or more nonsense mutations, one or more truncating mutations, and/or one or more essential splice site mutations. In some embodiments, a selection coefficient can be determined or calculated for one or more somatic mutations detected in one or more oncogenes and/or one or more tumor suppressor genes.

In step 440, the computer-readable medium comprises instructions that, when executed by the processor, cause the computer to compare the selection coefficients determined in step 430 for a locus, or for one or more loci, with a threshold value for detection of positive, neutral, or negative selection at the locus, or one or more loci. In one example, positive selection is detected when the threshold value is greater than 1 (i.e., when the selection coefficient is greater than 1) with a q-value less than 0.05. In another example, negative selection is detected when the threshold value is less than 1 (i.e., when the selection coefficient is less than 1) with a q-value less than 0.05. In still another example, neutral selection is detected when the selection coefficient is about 1.

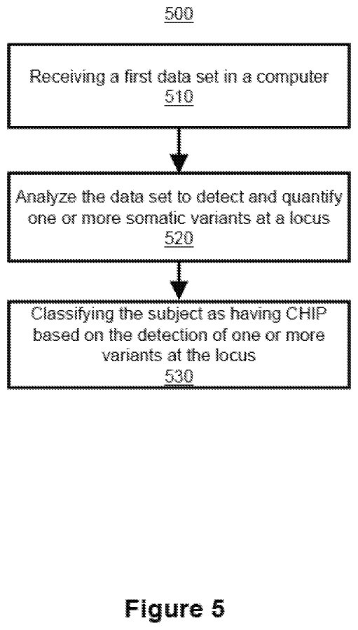

FIG. 5 is a flowchart illustrating a computer-implemented method 500 for classifying a subject as having clonal hematopoiesis of indeterminate potential (CHIP), in accordance with some embodiments of the present invention. Computer-implemented method 500 includes, but is not limited to, the following steps.

As shown at step 510, a first data set is received in a computer comprising a processor and a computer-readable medium, wherein the first data set comprises a plurality of sequence reads obtained by sequencing a plurality of cell-free nucleic acids in a test sample from a subject. In accordance with some embodiments, the computer-readable medium comprises instructions that, when executed by the processor, cause the computer carry out one or more data analysis steps. In some embodiments, the first data set is obtained from sequencing the test sample using whole-genome sequencing (WGS), whole-genome bisulfite sequencing (WGBS), whole-exome sequencing (WES), or from a targeted sequencing process. In some embodiments, the data can be obtained from a targeted sequencing process where the sequencing library has been enriched for one or more targeted nucleic acids prior to sequencing (as described above in conjunction with FIG. 1). The targeted enrichment step can utilize an enrichment panel comprising from about 10 to bout 10,000 targeted genes, about 50 to bout 1,000 genes, or about 100 to bout 500 genes. In another example, the first data set is obtained from sequencing the test sample using RNA sequencing (RNA-seq), transcriptome sequencing or whole-transcriptome shotgun sequencing (WTSS). In some embodiments, the sequencing data set is obtained from sequencing a sequencing library to a depth of at least 10×, at least 20×, at least 30×, at least 50×, or at least 100×. In some embodiments, the sequencing library is sequenced to a depth of at least 500×, at least 1,000×, at least 2,000×, at least 3,000×, or at least 10,000×.

At step 520, the computer-readable medium comprises instructions that, when executed by the processor, cause the computer to analyze the plurality of sequence reads to detect and quantify one or more somatic mutations at a locus known to be associated with or suspected of being associated with clonal hematopoiesis of indeterminate potential (CHIP). For example, DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B, or any combination thereof. Any known means in the art can be used to detect and quantify somatic mutations from the sequencing reads or sequencing data. For example, a variant calling pipeline can be used to detect and quantify somatic mutations or variants, as described in further detail below in conjunction with FIG. 13.

As described above in conjunction with FIG. 1, the one or more somatic mutations can comprise one or more single-nucleotide variants, or one or more small insertions and/or deletions (Indels). For example, the one or more somatic mutations comprise one or more nonsynonymous mutations, one or more missense mutations, one or more nonsense mutations, one or more truncating mutations, or one or more essential splice site mutations. In some embodiments, the one or more somatic mutations can be detected in one or more oncogenes or one or more tumor suppressor genes.

As previously described, sequence reads can be analyzed for identification of a known somatic mutation in a subject (e.g., a known somatic mutation associated with a disease or disease state) to assess or infer how a subject will respond to a therapeutic treatment targeting that somatic mutation. In some examples, sequence reads can be analyzed to identify previously unknown, or previously undetected somatic mutations (or variants) as potential targets for development of a therapeutic agent to treat a particular disease or disease state.

Optionally, in some embodiments, a selection coefficient is determined or calculated for each of the one or more loci where a somatic mutation (or variant) is detected. As previously described in conjunction with FIG. 1, in some embodiments, the selection coefficient is an estimate that directly enumerates the excess or deficit of mutations in a given loci, gene, group of genes, or even the whole exome (all genes) compared to the expectation for the background mutational process. For example, dN/dS (also referred to as Ka/Ks), as denoted by w, can be used to estimate the excess or deficit of mutations in a given loci, gene, group of genes, or even the whole exome (all genes) compared to the expectation for the background mutational process. As used herein, dN/dS (e.g., see Minyata and Yasunaga, J. Mol. Evol. (1980)) is the ratio between the rate of nonsynonymous per nonsynonymous site and synonymous substitutions per synonymous site. For somatic mutations (or variants), the rate of mutations per site can be used.

As previously described, dN/dS estimation for one or more somatic mutations can be modified to guard against confounding (see, e.g., Martincorena et al., Cell (2017)). For example, variation in mutation rate along the human genome can be taken into account. In another example, sequence context dependent mutational processes can be taken into account.

In one embodiment, a selection coefficient can be determined or calculated for one or more nonsynonymous mutations, one or more missense mutations, one or more nonsense mutations, one or more truncating mutations, and/or one or more essential splice site mutations. In another embodiment, a selection coefficient can be determined or calculated for one or more somatic mutations detected in one or more oncogenes and/or one or more tumor suppressor genes.

As step 530, the computer-readable medium comprises instructions that, when executed by the processor, cause the computer to classify the subject as having CHIP when one or more somatic mutations are detected at one or more loci known to be associated with or suspected of being associated with CHIP.

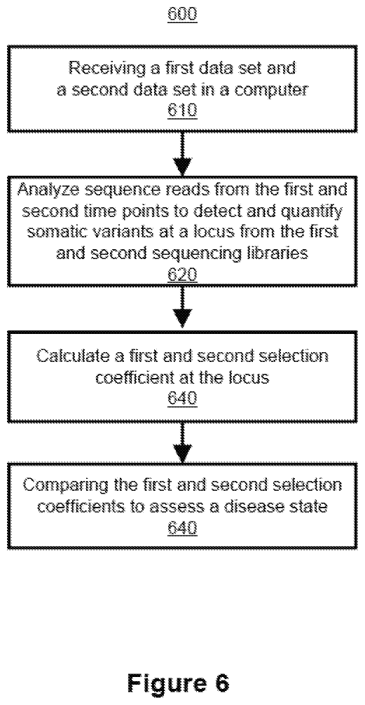

FIG. 6 is a flowchart illustrating a computer-implemented method 600 for assessing a disease state of a subject, in accordance with various embodiments of the present invention. Method 600 includes, but is not limited to, the following steps.

At step 610, sequencing data obtained from a first test sample, and sequencing data obtained from a second test sample, respectively, are received in a computer comprising a processor and a computer-readable medium. As described above in conjunction with FIG. 3, the test samples are obtained from a subject or patient suspected of having, at risk of having, or known to have a disease state. For example, a test sample may be obtained from a patient suspected of having, at risk of having, or known to have cancer, cardiovascular disease, a neurodegenerative disease, or other disease.

In some embodiments, the first and second sequencing data sets can be obtained from biological fluid samples selected from the group consisting of blood, plasma, serum, urine, saliva, fecal samples, and any combination thereof. In some embodiments, the test samples are biological test samples including one or more cells (e.g., blood cells). Still, in some embodiments, the test samples or biological test samples comprise a test sample selected from the group consisting of whole blood, a blood fraction, a tissue biopsy, pleural fluid, pericardial fluid, cerebrospinal fluid, peritoneal fluid, and any combination thereof. In some embodiments, the sample is a plasma sample from a cancer patient, or a patient suspected of having cancer. In accordance with some embodiments, the first and second test sample are blood samples, and the plurality of nucleic acids are genomic DNA (gDNA) molecules derived from blood cells (e.g., white blood cells (WBCs)). In accordance with one example, the first and second test samples are blood samples, and the plurality of nucleic acids are ribonucleic acid (RNA) molecules derived from blood cells (e.g., white blood cells (WBCs)). In accordance with still another example, the first and second test samples or biological test sample comprises a plurality of cell-free nucleic acids (e.g., cell-free DNA (cfDNA) and/or cell-free RNA (cfRNA)) fragments. In other examples, the test sample or biological test sample comprises a plurality of cell-free nucleic acid (e.g., cell-free DNA and RNA) fragments originating from healthy cells and from unhealthy cells (e.g., cancer cells). Optionally, the cell-free nucleic acids (e.g., cfDNA and/or cfRNA) can be extracted and/or purified from the test sample before proceeding with subsequent library preparation steps. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (Qiagen). In some examples, the sample can be a fragmented genomic DNA (gDNA) sample (e.g., a sheared gDNA sample).

In general, any time points could be used for collection of the first and second test samples and subsequent analysis. For example, the time points can be at least about 1 day apart, 2 days apart, 3 days apart, 4 days apart, 5 days apart, 6 days apart, 1 week apart, 1 month apart, 2 months apart, 3 months apart, 6 months apart, 7 months apart, 8 months apart, 9 months apart, 10 months apart, 11 months apart, or about 12 months apart. In another example, the time points can be semi-annually or annually, for example, every six months, one a year, once every other year, or once every two years. In still another example, the time points can be 1 year apart, 2 years apart, 3 years apart, 4 years apart, 5 years apart, 6 years apart, 7 years apart, 8 years apart, 9 years apart, or 10 years apart.

In some embodiments, method 600 includes collecting a plurality of biological samples at a plurality of time points and detecting and quantifying one or more features from each of the plurality of biological test samples obtained at the plurality of time points for monitoring the progression of a disease condition, or alternatively/additionally, the regression of a disease condition (e.g., in response to a treatment regimen and/or a therapeutic treatment for a disease condition). For example, 2 or more, 3 or more, 4 or more, 5 or more, 10 or more, 15 or more, 20 or more, or 2.5 or more test samples can be Obtained from a subject to monitor progression of a disease, regression of a disease, or to monitor response to a therapeutic agent to treat a disease.

At step 620, the computer-readable medium comprises instructions that, when executed by the processor, cause the computer to analyze the plurality of sequence reads obtained from the first sequencing library and the plurality of sequence reads obtained from the second sequencing library, respectively, to detect and quantify one or more somatic mutations from the first sequencing library at one or more loci and one or more somatic mutations from the second sequencing library at one or more loci, respectively. Any known means in the art can be used to detect and quantify somatic mutations from the sequencing reads or sequencing data. For example, a variant calling pipeline can be used to detect and quantify somatic mutations or variants, as described in further detail below in conjunction with FIG. 13.

In some embodiments, analyzing the plurality of sequence reads to detect and quantify the one or more somatic mutations applies a noise model, as described in further detail hereinbelow. In still other embodiments, analyzing the plurality of sequence reads further applies a read mis-mapping model to identify and account for artifactual somatic mutations (or variant) calls that may arise from mis-mapping. In one example, as described elsewhere herein, one or more white blood cell (WBC) matched cell-free DNA mutations (or variants) can be detected or identified in accordance with the present invention. For example, one or more somatic mutations are detected from joint modeling or mixture modeling both the cell-free nucleic acid somatic mutation and the one or more white blood cell derived somatic mutations.

In some embodiments, sequence reads covering one or more loci or genes known to be associated with a disease state can be analyzed to detect or identify a somatic mutation at that loci or genes. For example, one or more loci or genes known to be, or suspected of being, associated with cancer can be analyzed for somatic mutations (or variants) at those loci or genes. In another example, one or more loci or genes known to be associated, or suspected of being associated, with clonal hematopoiesis of indeterminate potential (CHIP) are analyzed. For example, DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B, or any combination thereof. In still another example, one or more loci or genes known to be, or suspected of being, associated with a neurodegenerative disease are analyzed. In another example, sequence reads can be analyzed for identification of a known somatic mutation in a subject (e.g., a known somatic mutation associated with a disease or disease state) to assess or infer how a subject will respond to a therapeutic treatment targeting that somatic mutation. In still another example, sequence reads can be analyzed to identify previously unknown, or previously undetected somatic mutations (or variants) as potential targets for development of a therapeutic agent to treat a particular disease or disease state.

In one example, the one or more somatic mutations comprise one or more single-nucleotide variants. In other examples, the one or more somatic mutations comprise one or more small insertions and/or deletions (Indels). For example, the one or more somatic mutations comprise one or more nonsynonymous mutations, one or more missense mutations, one or more nonsense mutations, one or more truncating mutations, or one or more essential splice site mutations. In another example, the one or more somatic mutations are detected in one or more oncogenes or one or more tumor suppressor genes.

At step 630, the computer-readable medium comprises instructions that, when executed by the processor, cause the computer to calculate a selection coefficient for each of the one or more loci where a somatic mutation (or variant) is detected from the first sequencing library and from the second sequencing library, respectively. In accordance with some embodiments of the present invention, the first and second selection coefficients are estimates that directly enumerate the excess or deficit of mutations in a given loci, gene, group of genes, or even the whole exome (all genes) compared to the expectation for the background mutational process. For example, dN/dS (also referred to as Ka/Ks), as denoted by w, can be used to estimate the excess or deficit of mutations in a given loci, gene, group of genes, or even the whole exome (all genes) compared to the expectation for the background mutational process. As used herein, dN/dS (e.g., see Minyata and Yasunaga, 1980) is the ratio between the rate of nonsynonymous per nonsynonymous site and synonymous substitutions per synonymous site. For somatic mutations (or variants), the rate of mutations per site can be used.

As previously described, dN/dS estimation for one or more somatic mutations can be modified to guard against confounding (see, e.g., Martincorena et al., Cell (2017)). For example, variation in mutation rate along the human genome can be taken into account. In another example, sequence context dependent mutational processes can be taken into account.

In one example, a selection coefficient can be determined or calculated for one or more nonsynonymous mutations, one or more missense mutations, one or more nonsense mutations, one or more truncating mutations, and/or one or more essential splice site mutations. In another example, a selection coefficient can be determined or calculated for one or more somatic mutations detected in one or more oncogenes and/or one or more tumor suppressor genes.

Finally, at step 640, the computer-readable medium comprises instructions that, when executed by the processor, cause the computer to compare the first selection coefficient and the second selection coefficient. In accordance with this embodiment, an increase in the second selection coefficient relative to the first coefficient indicates progression in the disease state, and a decrease in the second selection coefficient relative to the first selection coefficient indicates an improvement in the disease state.

### III. EXAMPLES

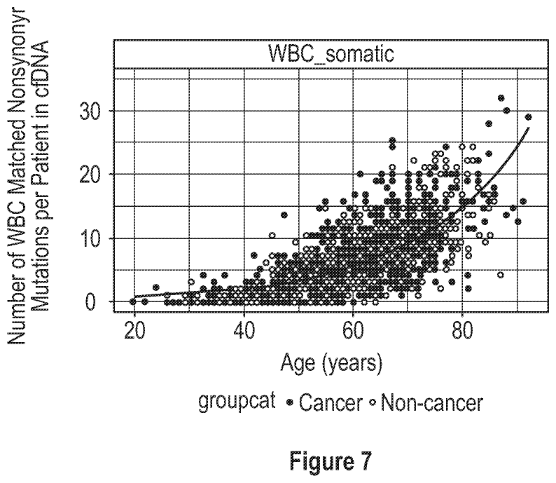

Example data shown in the following FIGS. 7-10 and FIGS. 33-34 were generated using sequence reads obtained from a sample set of individuals from GRAIL's circulating cell-free genome atlas (CCGA) study and processed using one or more of the methods described herein (including, for example, noise modeling, joint modeling, and/or edge filtering, as described hereinbelow). The sample set includes healthy individuals from which blood samples (e.g., cfDNA) were obtained. Additionally, the sample set includes individuals known to have at least one type of cancer, from which blood samples were obtained. The data was collected from individuals over approximately 1140 centers in the United States of America and Canada. FIGS. 7-10 show experimental results obtained from analysis of the sample set.

FIG. 7 is a dot plot showing the number of WBC matched nonsynonymous mutations detected at various ages per subject according to one embodiment. There were 9629 total nonsynonymous variants attributed to CHIP across 1438 individuals, median 5.5 per individual, (q25/q75 2, 10). The model suggests a constant rate (intercept) of 0.25 to 0.32 variants per individual. The estimated age effect per decade of 1.6 to 1.7 pval 0. Any additional interaction with healthy status for the rate by age is small 0.99 to 1 interaction pval 0.074. Adjusting by the expected number of variants and setting results at age 65, the predicted CHIP nonsynonymous burden is 7.1.

Table 1 shows the fraction of total WBC matched ctDNA variants detected at ≥10% variant allele frequency (VAF), at ≥1% VAF, and at ≥0.1% VAF. Prior art surveys of somatic variation in WBC across genes have currently been limited to low sequencing depth analyses (typically 100× depth or lower). However, as shown in Table 1 only approximately 1-2% of all WBC matched nonsynonymous mutations detected in the circulating cell-free genome atlas study have an allele frequency of greater than or equal to 10%. As such, low depth sequencing approaches do not account for the majority of somatic mutations in a biological test sample.

TABLE 1
Fraction of WBC-matched samples by frequency showing variant allele
frequency (VAF) distribution in cancer and non-cancer cohort.

| groupcat | VAF >= 10% | VAF >= 1% | VAF >= 0.1% |
|---|---|---|---|
| Cancer | 0.0097 | 0.093 | 0.87 |
| Non-cancer | 0.018 | 0.11 | 0.89 |

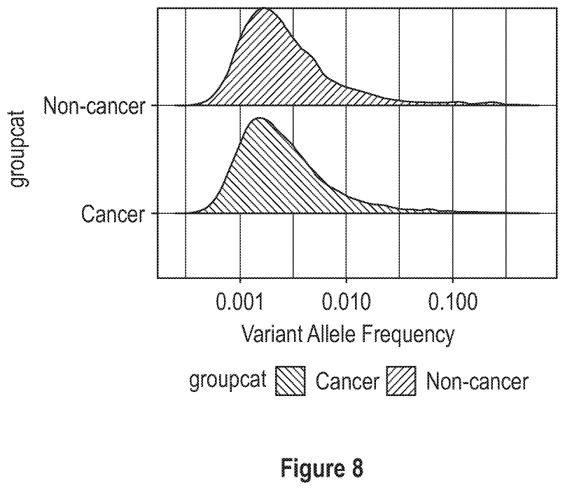

FIG. 8 is a plot showing variant allele frequencies of WBC-matched single nucleotide variants detected in healthy subjects (i.e., non-cancer) and subjects with cancer in the cancer CCGA study. As shown in FIG. 8, the majority of WBC matched cfDNA somatic variants detected were detected at an allele frequency below 0.01% in both healthy and cancer patients.

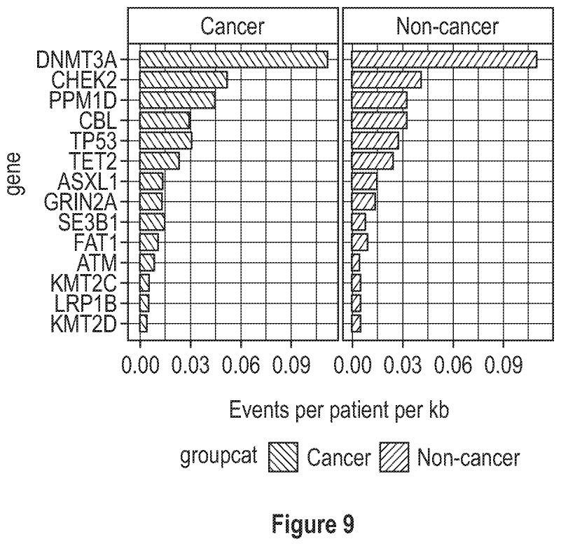

FIG. 9 is a plot showing the rate of mutation per kilobase for WBC-matched single nucleotide variants detected in various genes in accordance with various embodiments. The plot indicates that if WBC somatic variant contribution is not accounted for in small variant cfDNA assays, WBC somatic variants will confound cancer applications that use small variants as features. FIG. 9 represents mutations per kb across cancer and non-cancer displaying the top 20 genes (by rate).

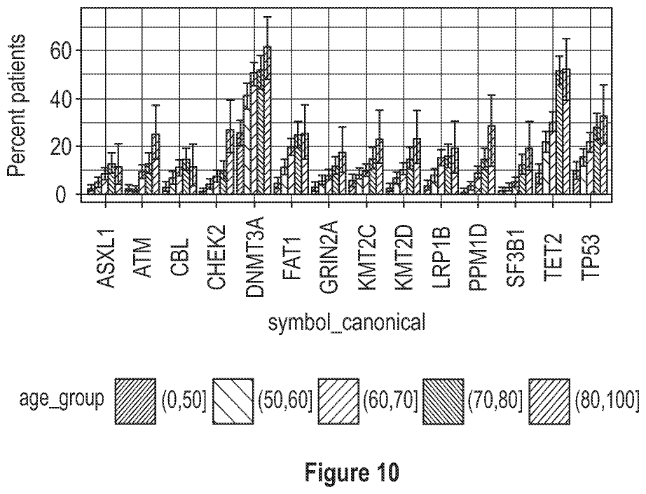

FIG. 10 is a plot showing the percentage of subjects where at least one WBC-matched mutation was detected for each of the various genes, in accordance with various embodiments. In this plot, variants from cancer and non-cancer participants have been combined for defined age groups. Age dependence is observed. Note for individuals between 60 and 70, 22% of individuals having a TP53 mutation (CI 18%-26%) is observed.

Table 2 shows selection coefficients calculated using dN/dS estimates for all missense (wmis) and nonsense (canon) mutations detected in the CCGA study. Coefficients>1 indicate positive selection across 507 genes.

TABLE 2
Selection coefficients for all somatic variants

| Selection coefficient | Estimate | LCB | UCB |
|---|---|---|---|
| omega_m | 1.37 | 1.306 | 1.436 |
| omega_n | 3.122 | 2.862 | 3.407 |

Per gene, selection coefficients show strong signals of positive selection after correcting for multiple testing across the 507 genes (BH correction). Note that differences in the magnitude and significance of missense and nonsense coefficients for each gene associate with oncogene and tumor suppressor gene activity.

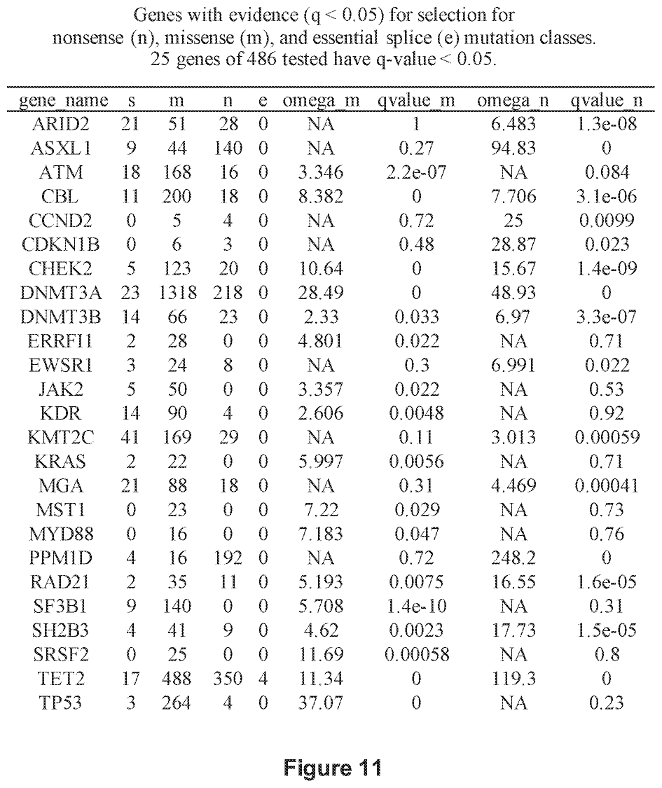

FIG. 11 is a table showing genes detected with evidence of selection (e.g., positive selection) using dN/dS estimates for all missense (wmis) and nonsense (wnon) mutations detected in the CCGA study across a 507 gene panel. Here, genes with evidence for selection for nonsense (n), missense (m), and essential splice (e) mutation classes have a q-value<0.05. Specifically, FIG. 11 shows that 25 genes of the 486 genes tested have q-value<0.05. The omega_m and omega_n column values indicate selection coefficients for missense and nonsense mutations, respectively. Higher values (e.g., values with greater magnitude) result in more of the variants being driven by selection.

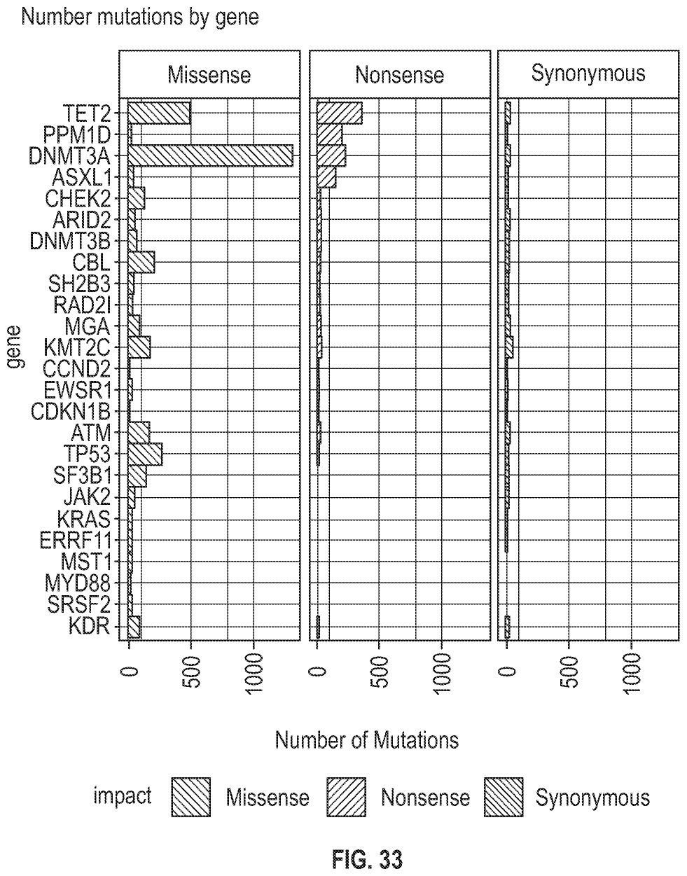

Turning now to FIG. 33, bar graphs show a number of missense, nonsense, and synonymous mutations by gene as detected in the CCGA study described herein. In some examples, a missense mutation results in a different amino acid being produced and therefore can change the function of a protein. In such cases, missense mutations can be considered as likely to lead to oncogenes that can transform a cell into a tumor cell. In some cases, genes having missense mutations can cause cancer when activated. On the other hand, in some examples, a nonsense mutation is a point mutation that turns one codon into a stop codon, thereby resulting in early termination of a polypeptide such that the protein is never completed. In such cases, genes with nonsense mutations can be tumor suppressor genes, which can cause cancer when inactivated. Further, in some examples, synonymous mutations do not result in significant changes in the protein.

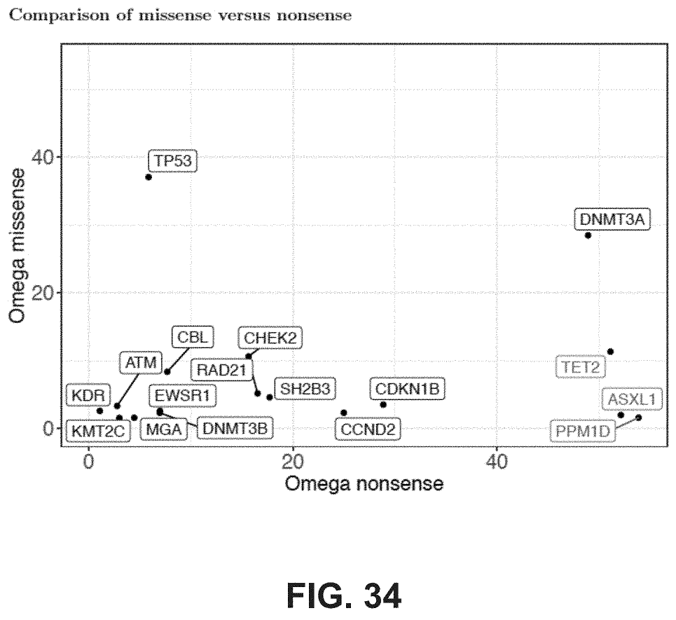

Turning to FIG. 34, a plot illustrates a comparison of nonsense and missense selection coefficients for various genes, as calculated in accordance with the systems and methods discussed herein. In some aspects, the plot indicates that variants detected in some genes (and in some cases, at specific locus or loci of the gene) are more likely driven by selection. (e.g., correspond to higher selection coefficients). For example, variants that are nonsense mutations that are detected in ASXL1, CDKN1B, DNMT3A, PPM1D, and TET2 genes correspond to high selection coefficients (e.g., along the x-axis), and thus are predicted as more likely to be driven by selection (e.g., positive selection) than other nonsense mutations at other genes that have lower selection coefficients along the x-axis. Similarly, the plot shows that variants that are missense mutations that are detected in TP53, DNMT3A, CHEK2, CBL, and TET2, genes correspond to high selection coefficients (e.g., along the y-axis), and therefore are predicted as more likely to be driven by selection (e.g., positive selection) than other missense mutations at other genes having lower selection coefficients along the y-axis.

It is noted that in some examples, a panel (e.g., sequencing assay panel) can be constructed by adding one or more genes that are identified as being indicative of positive selection when a certain mutation class (e.g., missense or nonsense mutation) is detected therein. In some cases, genes included in the panel are selected by ranking by magnitude a plurality of genes corresponding to a plurality of somatic variants based on their respective plurality of selection coefficients. Further, in some cases, panels can be specific for identifying missense mutations or nonsense mutations. For example, genes associated with missense mutations being indicative of positive correlation can be added to a panel to create a panel that detects for oncogenes. In some examples, genes associated with nonsense mutations being indicative of positive correlation can be added to a panel to create a panel that detects for tumor suppressor genes. Other examples are possible.

### IV. EXAMPLE PROCESSING SYSTEM

FIG. 12 is a block diagram of a processing system 1200 for processing sequence reads according to various embodiment. The processing system 1200 includes a sequence processor 1205, sequence database 1210, model database 1215, machine learning engine 1220, models 1225 (for example, including one or more Bayesian hierarchical models or joint models parameter database 1230, score engine 1235, variant caller 1240, edge filter 1250, and non-synonymous filter 1260. FIG. 13 is a flowchart of a method 1300 for determining variants of sequence reads according to various embodiments. In some embodiments, the processing system 1200 performs the method 1300 to perform variant calling (e.g., for SNVs and/or indels) based on input sequencing data. Further, the processing system 1200 can obtain the input sequencing data from an output file associated with nucleic acid sample prepared using various methods. The method 1300 includes, but is not limited to, the following steps, which are described with respect to the components of the processing system 1200. In some embodiments, one or more steps of the method 1300 are replaced by a step of a different process for generating variant calls, e.g., using Variant Call Format (VCF), such as HaplotypeCaller, VarScan, Strelka, or SomaticSniper.

At step 1300, the sequence processor 1205 collapses aligned sequence reads of the input sequencing data. In one example, collapsing sequence reads includes using UMIs, and optionally alignment position information from sequencing data of an output file to collapse multiple sequence reads into a consensus sequence for determining the most likely sequence of a nucleic acid fragment or a portion thereof. The unique sequence tag can be from about 4 to 20 nucleic acids in length. Because the UMIs are replicated with the ligated nucleic acid fragments through enrichment and PCR, the sequence processor 1205 can determine that certain sequence reads originated from the same molecule in a nucleic acid sample. In some embodiments, sequence reads that have the same or similar alignment position information (e.g., beginning and end positions within a threshold offset) and include a common UMI are collapsed, and the sequence processor 1205 generates a collapsed read (also referred to herein as a consensus read) to represent the nucleic acid fragment. The sequence processor 1205 designates a consensus read as “duplex” if the corresponding pair of collapsed reads have a common UMI, which indicates that both positive and negative strands of the originating nucleic acid molecule is captured; otherwise, the collapsed read is designated “non-duplex.” In some embodiments, the sequence processor 1205 can perform other types of error correction on sequence reads as an alternate to, or in addition to, collapsing sequence reads.

At step 1305, the sequence processor 1205 stitches the collapsed reads based on the corresponding alignment position information. In some embodiments, the sequence processor 1205 compares alignment position information between a first read and a second read to determine whether nucleotide base pairs of the first and second reads overlap in the reference genome. In one use case, responsive to determining that an overlap (e.g., of a given number of nucleotide bases) between the first and second reads is greater than a threshold length (e.g., threshold number of nucleotide bases), the sequence processor 1205 designates the first and second reads as “stitched”; otherwise, the collapsed reads are designated “unstitched.” In some embodiments, a first and second read are stitched if the overlap is greater than the threshold length and if the overlap is not a sliding overlap. For example, a sliding overlap can include a homopolymer run (e.g., a single repeating nucleotide base), a dinucleotide run (e.g., two-nucleotide base sequence), or a trinucleotide run (e.g., three-nucleotide base sequence), where the homopolymer run, dinucleotide fun, or trinucleotide run has at least a threshold length of base pairs.

At step 1310, the sequence processor 1205 assembles reads into paths. In some embodiments, the sequence processor 1205 assembles reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). The sequence processor 1205 aligns collapsed reads to a directed graph such that any of the collapsed reads can be represented in order by a subset of the edges and corresponding vertices.

In some embodiments, the sequence processor 1205 determines sets of parameters describing directed graphs and processes directed graphs. Additionally, the set of parameters can include a count of successfully aligned k-mers from collapsed reads to a k-mer represented by a node or edge in the directed graph. The sequence processor 1205 stores, e.g., in the sequence database 1210, directed graphs and corresponding sets of parameters, which can be retrieved to update graphs or generate new graphs. For instance, the sequence processor 1205 can generate a compressed version of a directed graph (e.g., or modify an existing graph) based on the set of parameters. In one use case, in order to filter out data of a directed graph having lower levels of importance, the sequence processor 1205 removes (e.g., “trims” or “prunes”) nodes or edges having a count less than a threshold value, and maintains nodes or edges having counts greater than or equal to the threshold value.

At step 1315, the variant caller 1240 generates candidate variants from the paths assembled by the sequence processor 1205. In one embodiment, the variant caller 1240 generates the candidate variants by comparing a directed graph (which could have been compressed by pruning edges or nodes in step 1310) to a reference sequence of a target region of a genome. The variant caller 1240 can align edges of the directed graph to the reference sequence, and records the genomic positions of mismatched edges and mismatched nucleotide bases adjacent to the edges as the locations of candidate variants. In some embodiments, the genomic positions of mismatched edges and mismatched nucleotide bases to the left and right of edges are recorded as the locations of called variants. Additionally, the variant caller 1240 can generate candidate variants based on the sequencing depth of a target region. In particular, the variant caller 1240 can be more confident in identifying variants in target regions that have greater sequencing depth, for example, because a greater number of sequence reads help to resolve (e.g., using redundancies) mismatches or other base pair variations between sequences.

In some embodiments, the variant caller 1240 generates candidate variants using a model 1225 to determine expected noise rates for sequence reads from a subject. The model 1225 may be a Bayesian hierarchical model, though in some embodiments, the processing system 1200 uses one or more different types of models. Moreover, a Bayesian hierarchical model can be one of many possible model architectures that can be used to generate candidate variants and which are related to each other in that they all model position-specific noise information in order to improve the sensitivity/specificity of variant calling. More specifically, the machine learning engine 1220 trains the model 1225 using samples from healthy individuals to model the expected noise rates per position of sequence reads.

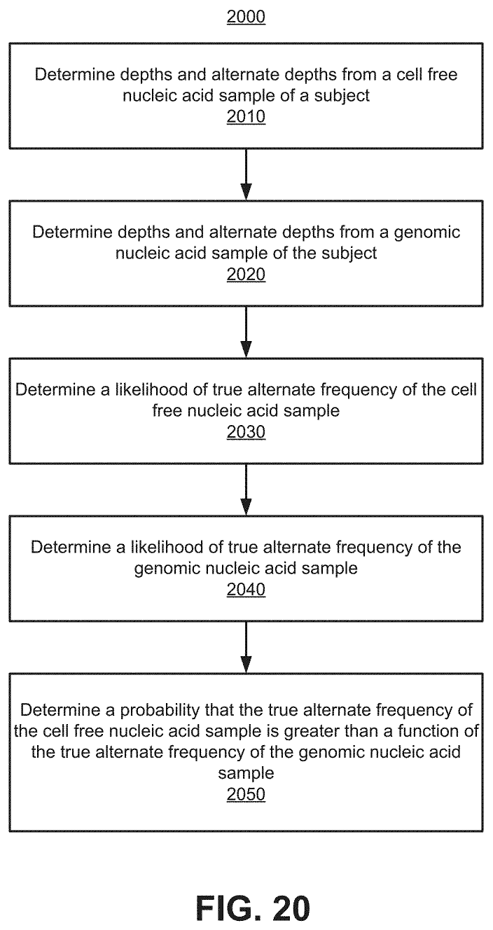

Further, multiple different models can be stored in the model database 1215 or retrieved for application post-training. For example, a first model is trained to model SNV noise rates and a second model is trained to model indel noise rates. Further, the score engine 1235 can use parameters of the model 1225 to determine a likelihood of one or more true positives in a sequence read. The score engine 1235 can determine a quality score (e.g., on a logarithmic scale) based on the likelihood. For example, the quality score is a Phred quality score Q=10·log₁₀ P, where P is the likelihood of an incorrect candidate variant call (e.g., a false positive). Other models 1225 such as a joint model (further described below with reference to FIGS. 20-25) can use output of one or more Bayesian hierarchical models to determine expected noise of nucleotide mutations in sequence reads of different samples.

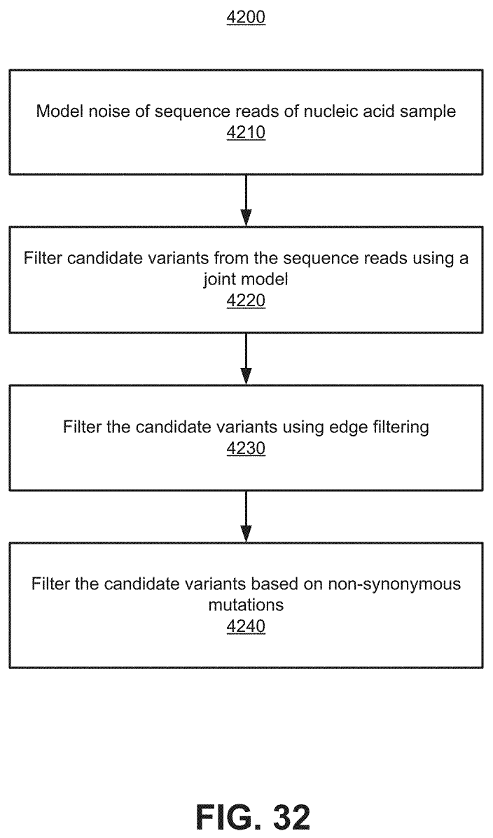

At step 1320, the processing system 1200 filters the candidate variants using one or more types of models 1225 or filters. For example, the score engine 1235 scores the candidate variants using a joint model, edge variant prediction model, or corresponding likelihoods of true positives or quality scores. In addition, the processing system 1200 can filter edge variants and/or non-synonymous mutations using the edge filter 1250 and/or nonsynonymous filter 1260, respectively. Training and application of these models and filters are described in more detail throughout the specification, and in particular with reference to FIG. 32.

At step 1325, the processing system 1200 outputs the filtered candidate variants. In some embodiments, the processing system 1200 outputs some or all of the determined candidate variants along with corresponding one scores from the filtering steps. Downstream systems, e.g., external to the processing system 1200 or other components of the processing system 1200, can use the candidate variants and scores for various applications including, but not limited to, predicting presence of cancer, disease, or germline mutations.

### V. EXAMPLE NOISE MODELS

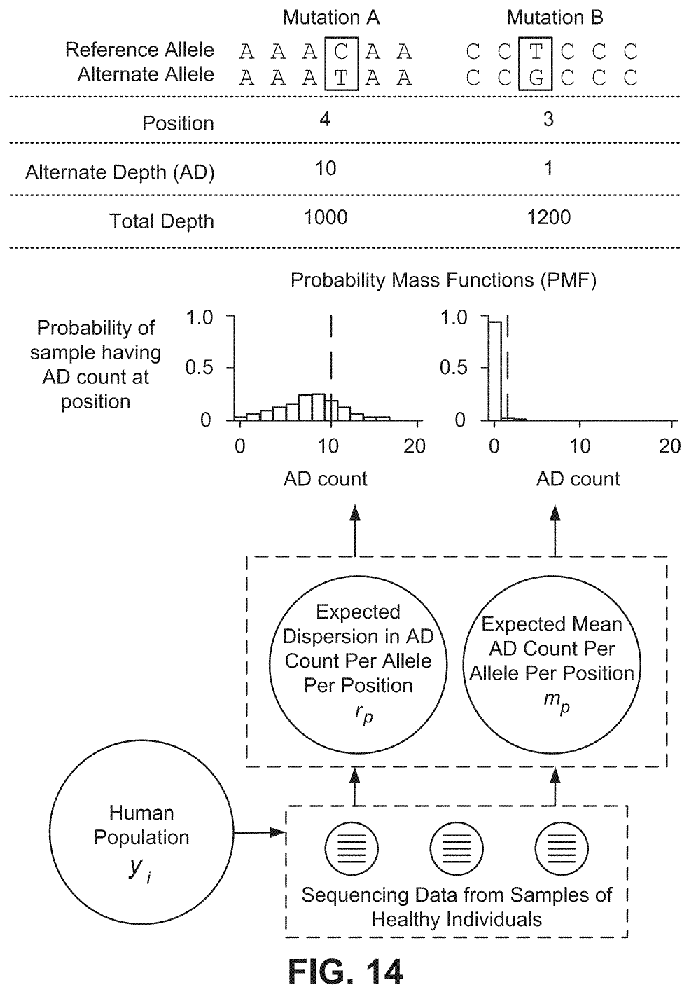

FIG. 14 is a diagram of an application of a Bayesian hierarchical model 1225 according to some embodiments. Mutation A and Mutation B are shown as examples for purposes of explanation. In the example at FIG. 14, Mutations A and B are represented as SNVs, though in other embodiments, the following description is also applicable to indels or other types of mutations. Mutation A is a C>T mutation at position 4 of a first reference allele from a first sample. The first sample has a first AD of 10 and a first total depth of 1000. Mutation B is a T>G mutation at position 3 of a second reference allele from a second sample. The second sample has a second AD of 1 and a second total depth of 1200. Based merely on AD (or AF), Mutation A may appear to be a true positive, while Mutation B may appear to be a false positive because the AD (or AF) of the former is greater than that of the latter. However, Mutations A and B may have different relative levels of noise rates per allele and/or per position of the allele. In fact, Mutation A may be a false positive and Mutation B may be a true positive, once the relative noise levels of these different positions are accounted for. The models 1225 described herein model this noise for appropriate identification of true positives accordingly.

The probability mass functions (PMFs) illustrated in FIG. 14 indicate the probability (or likelihood) of a sample from a subject having a given AD count at a position. Using sequencing data from samples of healthy individuals (e.g., stored in the sequence database 1210), the processing system 1200 trains a model 1225 from which the PMFs for healthy samples may be derived. In particular, the PMFs are based on mₚ, which models the expected mean AD count per allele per position in normal tissue (e.g., of a healthy individual), and rₚ, which models the expected variation (e.g., dispersion) in this AD count. Stated differently, mₚ and/or rₚ represent a baseline level of noise, on a per position per allele basis, in the sequencing data for normal tissue.

Using the example of FIG. 14 to further illustrate, samples from the healthy individuals represent a subset of the human population modeled by yᵢ, where i is the index of the healthy individual in the training set. Assuming for the sake of example the model 1225 has already been trained, PMFs produced by the model 1225 visually illustrate the likelihood of the measured ADs for each mutation, and therefore provide an indication of which are true positives and which are false positives. The example PMF on the left of FIG. 14 associated with Mutation A indicates that the probability of the first sample having an AD count of 10 for the mutation at position 4 is approximately 20%. Additionally, the example PMF on the right associated with Mutation B indicates that the probability of the second sample having an AD count of 1 for the mutation at position 3 is approximately 1% (note: the PMFs of FIG. 14 are not exactly to scale). Thus, the noise rates corresponding to these probabilities of the PMFs indicate that Mutation A is more likely to occur than Mutation B, despite Mutation B having a lower AD and AF. Thus, in this example, Mutation B may be the true positive and Mutation A may be the false positive. Accordingly, the processing system 1200 may perform improved variant calling by using the model 1225 to distinguish true positives from false positives at a more accurate rate, and further provide numerical confidence as to these likelihoods.

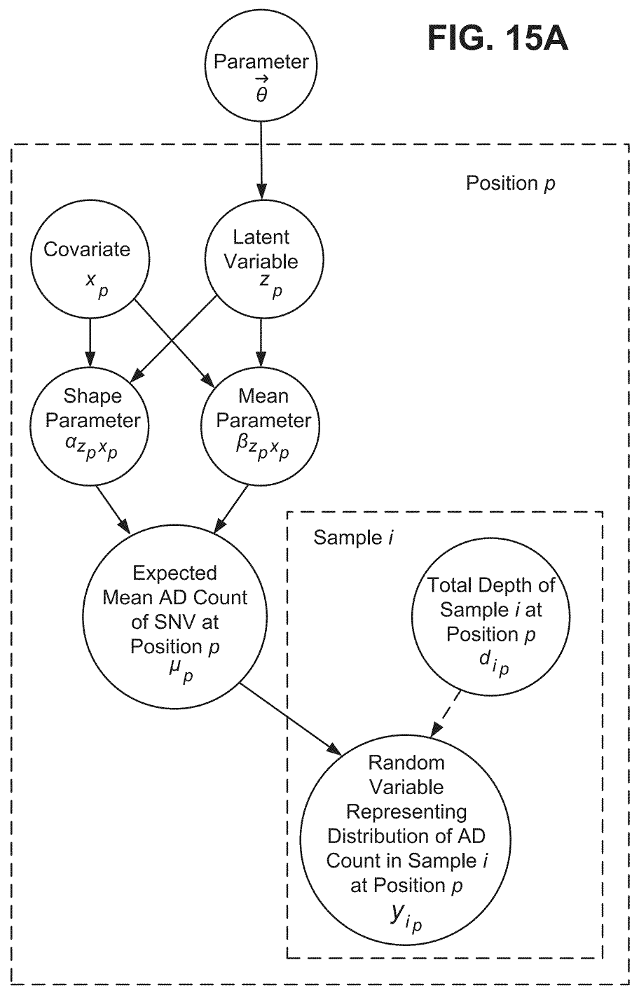

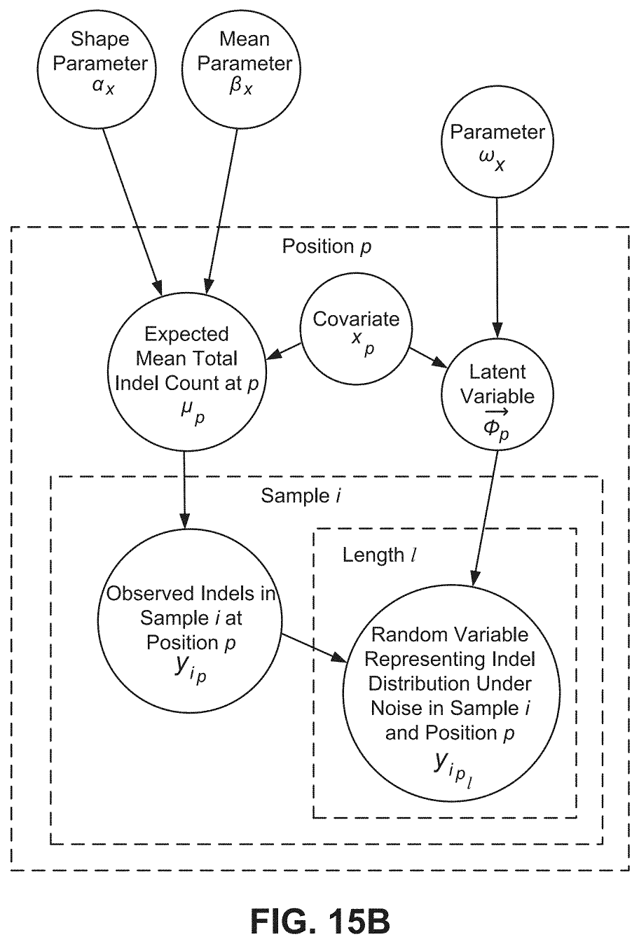

FIG. 15A shows dependencies between parameters and sub-models of a Bayesian hierarchical model 1225 for determining true single nucleotide variants according to one embodiment. Parameters of models can be stored in the parameter database 1230. In the example shown in FIG. 15A, \vec{θ} represents the vector of weights assigned to each mixture component. The vector \vec{θ} takes on values within the simplex in K dimensions and can be learned or updated via posterior sampling during training. It can be given a uniform prior on said simplex for such training. The mixture component to which a position p belongs can be modeled by latent variable zₚ using one or more different multinomial distributions:

zₚ˜Multinom(\vec{θ})

Together, the latent variable zₚ, the vector of mixture components \vec{θ}, α, and β allow the model for μ, that is, a sub-model of the Bayesian hierarchical model 1225, to have parameters that “pool” knowledge about noise, that is they represent similarity in noise characteristics across multiple positions. Thus, positions of sequence reads can be pooled or grouped into latent classes by the model. Also advantageously, samples of any of these “pooled” positions can help train these shared parameters. A benefit of this is that the processing system 1200 can determine a model of noise in healthy samples even if there is little to no direct evidence of alternative alleles having been observed for a given position previously (e.g., in the healthy tissue samples used to train the model).

The covariate xₚ (e.g., a predictor) encodes known contextual information regarding position p which can include, but is not limited to, information such as trinucleotide context, mappability, segmental duplication, or other information associated with sequence reads. Trinucleotide context can be based on a reference allele and can be assigned numerical (e.g., integer) representation. For instance, “AAA” is assigned 1, “ACA” is assigned 2, “AGA” is assigned 3, etc. Mappability represents a level of uniqueness of alignment of a read to a particular target region of a genome. For example, mappability is calculated as the inverse of the number of position(s) where the sequence read will uniquely map. Segmental duplications correspond to long nucleic acid sequences (e.g., having a length greater than approximately 1000 base pairs) that are nearly identical (e.g., greater than 90% match) and occur in multiple locations in a genome as result of natural duplication events (e.g., not associated with a cancer or disease).

The expected mean AD count of a SNV at position p is modeled by the parameter μₚ. For sake of clarity in this description, the terms μₚ and yₚ refer to the position specific sub-models of the Bayesian hierarchical model 1225. In one embodiment, μₚ is modeled as a Gamma-distributed random variable having shape parameter α_{z}_{ₚ}_{,x}_{ₚ} and mean parameter

β_{z}_{ₚ}_{,x}_{ₚ}:μₚ˜Gamma(α_{z}_{ₚ}_{,x}_{ₚ}, β_{z}_{ₚ}_{,x}_{ₚ})

In some embodiments, other functions can be used to represent μₚ, examples of which include but are not limited to: a log-normal distribution with log-mean y_{z}_{ₚ} and log-standard-deviation σ_{z}_{ₚ}ₓ_{ₚ}, a Weibull distribution, a power law, an exponentially-modulated power law, or a mixture of the preceding.

In the example shown in FIG. 15A, the shape and mean parameters are each dependent on the covariate xₚ and the latent variable zₚ, though in other embodiments, the dependencies may be different based on various degrees of information pooling during training. For instance, the model can alternately be structured so that α_{z}_{ₚ} depends on the latent variable but not the covariate. The distribution of AD count of the SNV at position p in a human population sample i (of a healthy individual) is modeled by the random variable yᵢₚ. In one example, the distribution is a Poisson distribution given a depth dᵢₚ of the sample at the position:

yᵢₚ|dᵢ_{ₚ}˜Poisson(dᵢ_{ₚ}·μₚ)

In some embodiments, other functions can be used to represent yᵢₚ, examples of which include but are not limited to: negative binomial, Conway-Maxwell-Poisson distribution, zeta distribution, and zero-inflated Poisson.

FIG. 15B shows dependencies between parameters and sub-models of a Bayesian hierarchical model for determining true insertions or deletions according to some embodiments. In contrast to the SNV model shown in FIG. 15A, the model for indels shown in FIG. 15B includes different levels of hierarchy. The covariate xₚ encodes known features at position p and can include, e.g., a distance to a homopolymer, distance to a RepeatMasker repeat, or other information associated with previously observed sequence reads. Latent variable \vec{ϕ}ₚ can be modeled by a Dirichlet distribution based on parameters of vector \vec{ω}ₓ, which represent indel length distributions at a position and can be based on the covariate. In some embodiments, \vec{ω}ₓ is also shared across positions (\vec{ω}_{x,p}) that share the same covariate value(s). Thus for example, the latent variable can represent information such as that homopolymer indels occur at positions 1, 3, etc. base pairs from the anchor position, while trinucleotide indels occur at positions 3, 6, 9, etc. from the anchor position.

The expected mean total indel count at position p is modeled by the distribution μₚ. In some embodiments, the distribution is based on the covariate and has a Gamma distribution having shape parameter αₓ_{ₚ} and mean parameter

βₓ_{ₚ}_{z}_{ₚ}:μₚ˜Gamma(αₓ_{ₚ}, βₓ_{ₚ}_{z}_{ₚ})

In some embodiments, other functions can be used to represent μₚ, examples of which include but are not limited to: negative binomial, Conway-Maxwell-Poisson distribution, zeta distribution, and zero-inflated Poisson.

The observed indels at position p in a human population sample i (of a healthy individual) is modeled by the distribution yᵢₚ. Similar to the example in FIG. 15A, in some embodiments, the distribution of indel intensity is a Poisson distribution given a depth dᵢ_{ₚ} of the sample at the position:

ᵢₚ|dᵢ_{ₚ}˜Poisson(dᵢ_{ₚ}·μₚ)

In some embodiments, other functions can be used to represent yᵢₚ, examples of which include but are not limited to: negative binomial, Conway-Maxwell-Poisson distribution, zeta distribution, and zero-inflated Poisson.

Due to the fact that indels can be of varying lengths, an additional length parameter is present in the indel model that is not present in the model for SNVs. As a consequence, the example model shown in FIG. 15B has an additional hierarchical level (e.g., another sub-model), which is again not present in the SNV models discussed above. The observed count of indels of length l (e.g., up to 100 or more base pairs of insertion or deletion) at position p in sample i is modeled by the random variable yᵢₚ_{₁}, which represents the indel distribution under noise conditional on parameters. The distribution can be a multinomial given indel intensity yᵢₚ of the sample and the distribution of indel lengths \vec{ϕ}ₚ at the position:

\vec{y}ᵢₚ_{₁}|yᵢₚ, \vec{ϕ}ₚ·Multinom(yᵢ_{ₚ}, \vec{ϕ}ₚ)

In some embodiments, a Dirichlet-Multinomial function or other types of models can be used to represent yᵢₚ_{₁}.

By architecting the model in this manner, the machine learning engine 1220 can decouple learning of indel intensity (i.e., noise rate) from learning of indel length distribution. Independently determining inferences for an expectation for whether an indel will occur in healthy samples and expectation for the length of the indel at a position can improve the sensitivity of the model. For example, the length distribution can be more stable relative to the indel intensity at a number of positions or regions in the genome, or vice versa.

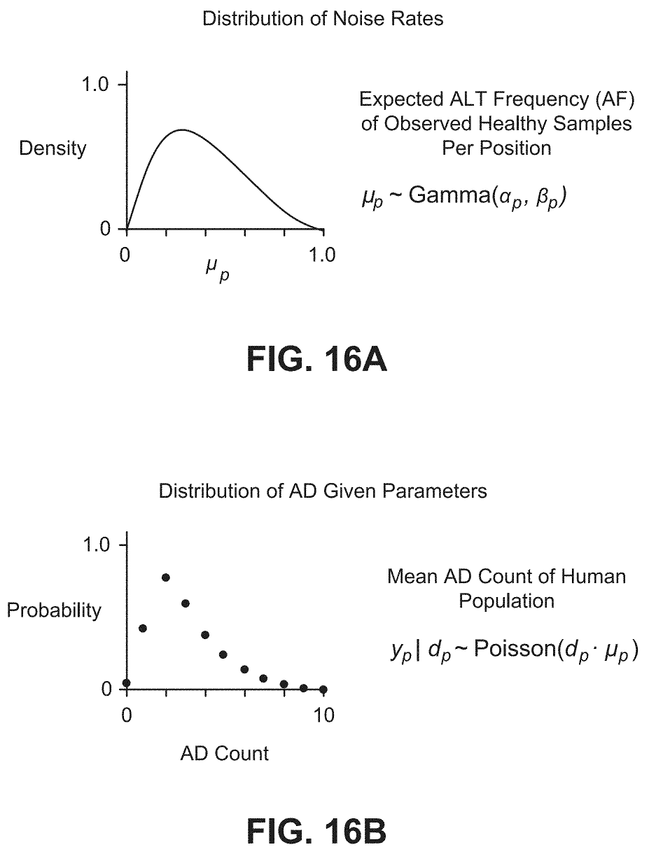

FIGS. 16A-B illustrate diagrams associated with a Bayesian hierarchical model 1225 according to some embodiments. The graph shown in FIG. 16A depicts the distribution μₚ of noise rates, i.e., likelihood (or intensity) of SNVs or indels for a given position as characterized by a model. The continuous distribution represents the expected AF μₚ of non-cancer or non-disease mutations (e.g., mutations naturally occurring in healthy tissue) based on training data of observed healthy samples from healthy individuals (e.g., retrieved from the sequence database 1210). Though not shown in FIG. 16A, in some embodiments, the shape and mean parameters of μₚ can be based on other variables such as the covariate xₚ or latent variable zₚ. The graph shown in FIG. 16B depicts the distribution of AD at a given position for a sample of a subject, given parameters of the sample such as sequencing depth dₚ at the given position. The discrete probabilities for a draw of μₚ are determined based on the predicted true mean AD count of the human population based on the expected mean distribution μₚ.

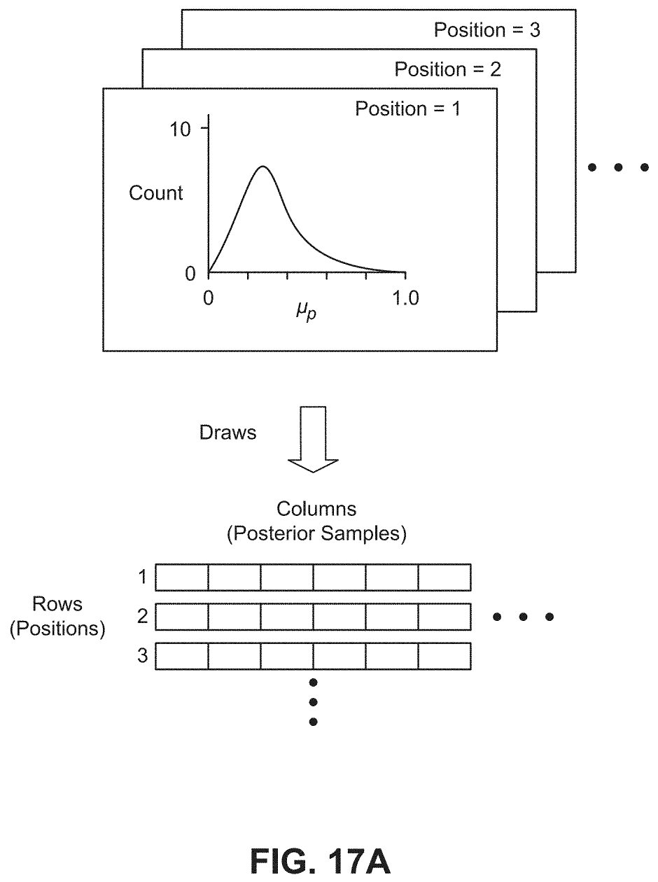

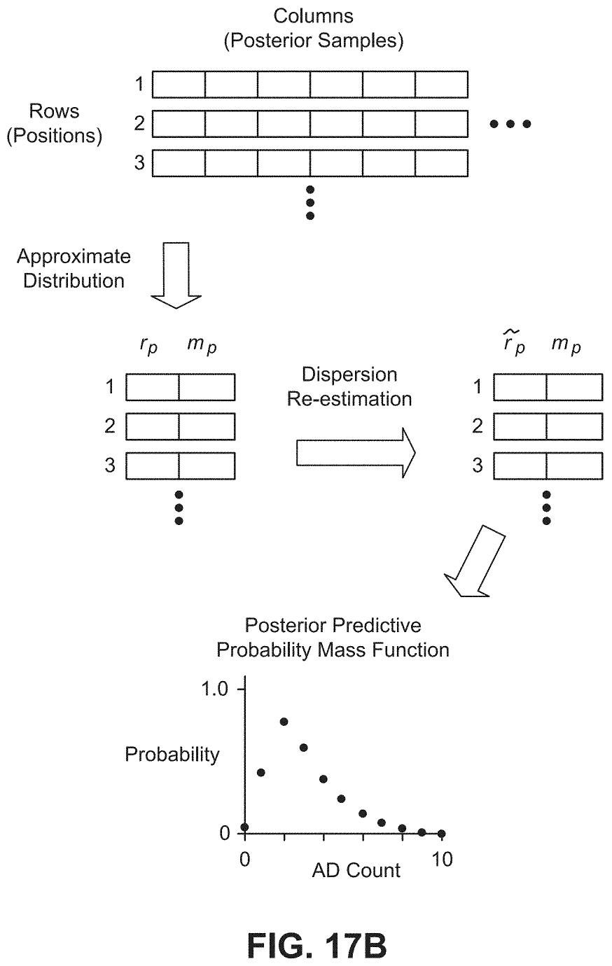

FIG. 17A is a diagram of an example process for determining parameters by fitting a Bayesian hierarchical model 1225 according to some embodiments. To train a model, the machine learning engine 1220 samples iteratively from a posterior distribution of expected noise rates (e.g., the graph shown in FIG. 16B) for each position of a set of positions. The machine learning engine 1220 can use Markov chain Monte Carlo (MCMC) methods for sampling, e.g., a Metropolis-Hastings (MH) algorithm, custom MH algorithms, Gibbs sampling algorithm, Hamiltonian mechanics-based sampling, random sampling, among other sampling algorithms. During Bayesian Inference training, parameters are drawn from the joint posterior distribution to iteratively update all (or some) parameters and latent variables of the model (e.g., \vec{θ}, zₚ, α_{z}_{ₚ}_{,x}_{ₚ}, β_{z}_{ₚ}_{,x}_{ₚ}, μₚ, etc.).

In some embodiments, the machine learning engine 1220 performs model fitting by storing draws of μₚ, the expected mean counts of AF per position and per sample, in the parameters database 1230. The model is trained or fitted through posterior sampling, as previously described. In an example, the draws of μₚ are stored in a matrix data structure having a row per position of the set of positions sampled and a column per draw from the joint posterior of all parameters conditional on the observed data). The number of rows R may be greater than 6 million and the number of columns for N iterations of samples may be in the thousands. In other examples, the row and column designations are different than the embodiment shown in FIG. 17A, e.g., each row represents a draw from a posterior sample, and each column represents a sampled position (e.g., transpose of the matrix example shown in FIG. 17A).

FIG. 17B is a diagram of using parameters from a Bayesian hierarchical model 1225 to determine a likelihood of a false positive according to one embodiment. The machine learning engine 1220 may reduce the R rows-by-N column matrix shown in FIG. 17A into an R rows-by-2 column matrix illustrated in FIG. 17B. In some embodiments, the machine learning engine 1220 determines a dispersion parameter rₚ (e.g., shape parameter) and mean parameter mₚ (which may also be referred to as a mean rate parameter mₚ) per position across the posterior samples μₚ. The dispersion parameter rₚ can be determined as

$${r}_{p} = \frac{{m}_{p}^{2}}{{v}_{p}^{2}} ,$$

where mₚ and vₚ are the mean and variance of the sampled values of μₚ at the position, respectively. Those of skill in the art will appreciate that other functions for determining rₚ can also be used such as a maximum likelihood estimate.

The machine learning engine 1220 can also perform dispersion re-estimation of the dispersion parameters in the reduced matrix, given the mean parameters. In one example, following Bayesian training and posterior approximation, the machine learning engine 1220 performs dispersion re-estimation by retraining for the dispersion parameters \tilde{r}ₚ based on a negative binomial maximum likelihood estimator per position. The mean parameter can remain fixed during retraining. In one example, the machine learning engine 1220 determines the dispersion parameters r′ₚ at each position for the original Al) counts of the training data (e.g., yᵢₚ and dᵢ_{ₚ} based on healthy samples). The machine learning engine 1220 determines \tilde{r}ₚ=max(rₚ, r′ₚ), and stores \tilde{r}ₚ in the reduced matrix. Those of skill in the art will appreciate that other functions for determining \tilde{r}ₚ can also be used, such as a method of moments estimator, posterior mean, or posterior mode.

During application of trained models, the processing system 1200 can access the dispersion (e.g., shape) parameters \tilde{r}ₚ and mean parameters in to determine a function parameterized by \tilde{r}ₚ and mₚ. The function can be used to determine a posterior predictive probability mass function (or probability density function) for a new sample of a subject. Based on the predicted probability of a certain AD count at a given position, the processing system 1200 can account for site-specific noise rates per position of sequence reads when detecting true positives from samples. Referring back to the example use case described with respect to FIG. 14, the PMFs shown for Mutations A and B can be determined using the parameters from the reduced matrix of FIG. 17B. The posterior predictive probability mass functions can be used to determine the probability of samples for Mutations A or B having an AD count at certain position.

### VI. EXAMPLE PROCESS FLOWS FOR NOISE MODELS

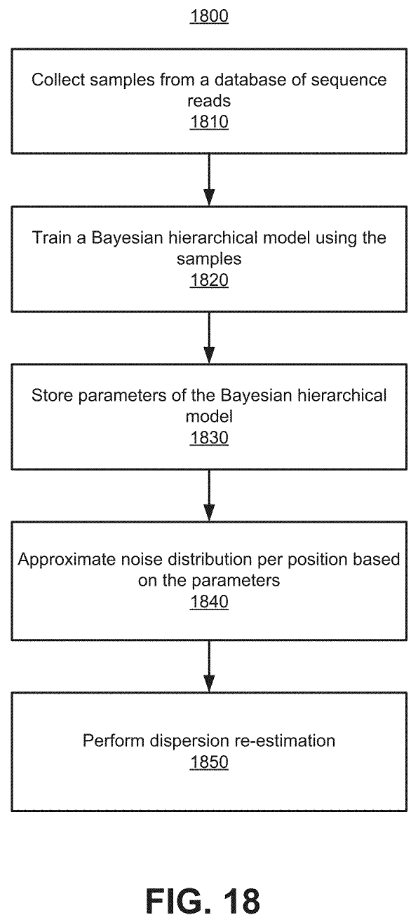

FIG. 18 is a flowchart of a method 1800 for training a Bayesian hierarchical model 1225 according to some embodiments. In step 1810, the machine learning engine 1220 collects samples, e.g., training data, from a database of sequence reads (e.g., the sequence database 1210). In step 1820, the machine learning engine 1220 trains the Bayesian hierarchical model 1225 using the samples using a Markov Chain Monte Carlo method. During training, the model 1225 can keep or reject sequence reads conditional on the training data. The machine learning engine 1220 can exclude sequence reads of healthy individuals that have less than a threshold depth value or that have an AF greater than a threshold frequency in order to remove suspected germline mutations that are not indicative of target noise in sequence reads. In other embodiments, the machine learning engine 1220 can determine which positions are likely to contain germline variants and selectively exclude such positions using thresholds like the above. In one example, the machine learning engine 1220 can identify such positions as having a small mean absolute deviation of AFs from germline frequencies (e.g., 0, ½, and 1).

The Bayesian hierarchical model 1225 can update parameters simultaneously for multiple (or all) positions included in the model. Additionally, the model 1225 can be trained to model expected noise for each ALT. For instance, a model for SNVs can perform a training process four or more times to update parameters (e.g., one-to-one substitutions) for mutations of each of the A, T, C, and G bases to each of the other three bases. In step 1830, the machine learning engine 1220 stores parameters of the Bayesian hierarchical model 1225 (e.g., ensemble parameters output by the Markov Chain Monte Carlo method). In step 1840, the machine learning engine 1220 approximates noise distribution (e.g., represented by a dispersion parameter and a mean parameter) per position based on the parameters. In step 1850, the machine learning engine 1220 performs dispersion re-estimation (e.g., maximum likelihood estimation) using original AD counts from the samples (e.g., training data) used to train the Bayesian hierarchical model 1225.

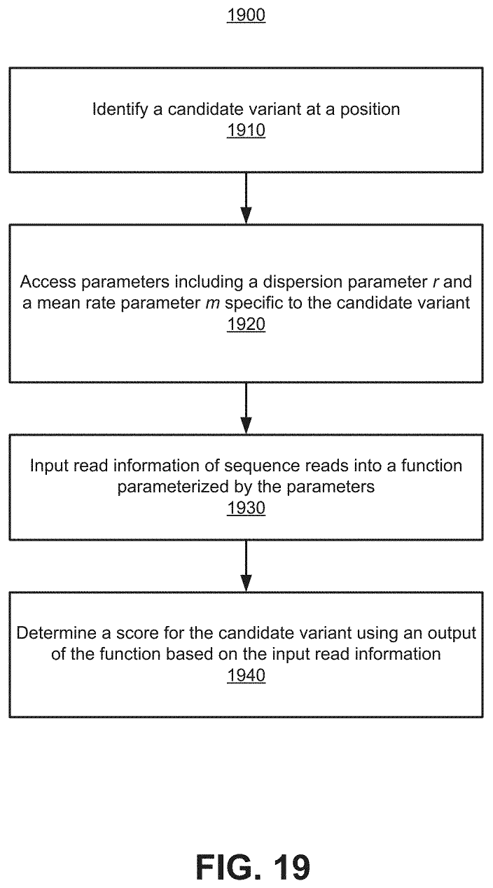

FIG. 19 is a flowchart of a method 1900 for determining a likelihood of a false positive according to various embodiments. At step 1910, the processing system 1200 identifies a candidate variant, e.g., at a position p of a sequence read, from a set of sequence reads, which can be sequenced from a cfDNA sample obtained from an individual. At step 1920, the processing system 1200 accesses parameters, e.g., dispersion and mean rate parameters \tilde{r}ₚ and mₚ, respectively, specific to the candidate variant, which can be based on the position p of the candidate variant. The parameters can be derived using a model, e.g., a Bayesian hierarchical model 1225 representing a posterior predictive distribution with an Observed depth of a given sequence read and a mean parameter μₚ at the position p as input. In an embodiment, the mean parameter μₚ is a gamma distribution encoding a noise level of nucleotide mutations with respect to the position p for a training sample.

At step 1930, the processing system 1200 inputs read information (e.g., AD or AF) of the set of sequence reads into a function (e.g., based on a negative binomial) parameterized by the parameters, e.g., \tilde{r}ₚ and mₚ. At step 1940, the processing system 1200 (e.g., the score engine 1235) determines a score for the candidate variant (e.g., at the position p) using an output of the function based on the input read information. The score may indicate a likelihood of seeing an allele count for a given sample (e.g., from a subject) that is greater than or equal to a determined allele count of the candidate variant (e.g., determined by the model and output of the function). The processing system 1200 can convert the likelihood into a Phred-scaled score. In some embodiments, the processing system 1200 uses the likelihood to determine false positive mutations responsive to determining that the likelihood is less than a threshold value. In some embodiments, the processing system 1200 uses the function to determine that a sample of sequence reads includes at least a threshold count of alleles corresponding to a gene found in sequence reads from a tumor biopsy of an individual. Responsive to this determination, the processing system 1200 can predict presence of cancer cells in the individual based on variant calls. In some embodiments, the processing system 1200 can perform weighting based on quality scores, use the candidate variants and quality scores for false discovery methods, annotate putative calls with quality scores, or provision to subsequent systems.

The processing system 1200 can use functions encoding noise levels of nucleotide mutations with respect to a given training sample for downstream analysis. In some embodiments, the processing system 1200 uses the aforementioned negative binomial function parameterized by the dispersion and mean rate parameters \tilde{r}ₚ and mₚ to determine expected noise for a particular nucleic acid position within a sample, e.g., gDNA or gDNA. Moreover, the processing system 1200 can derive the parameters by training a Bayesian hierarchical model 1225 using training data associated with the particular nucleic acid sample. The embodiments below describe another type of model referred to herein as a joint model 1225, which can use the output of a Bayesian hierarchical model 1225.

### VII. EXAMPLE JOINT MODEL

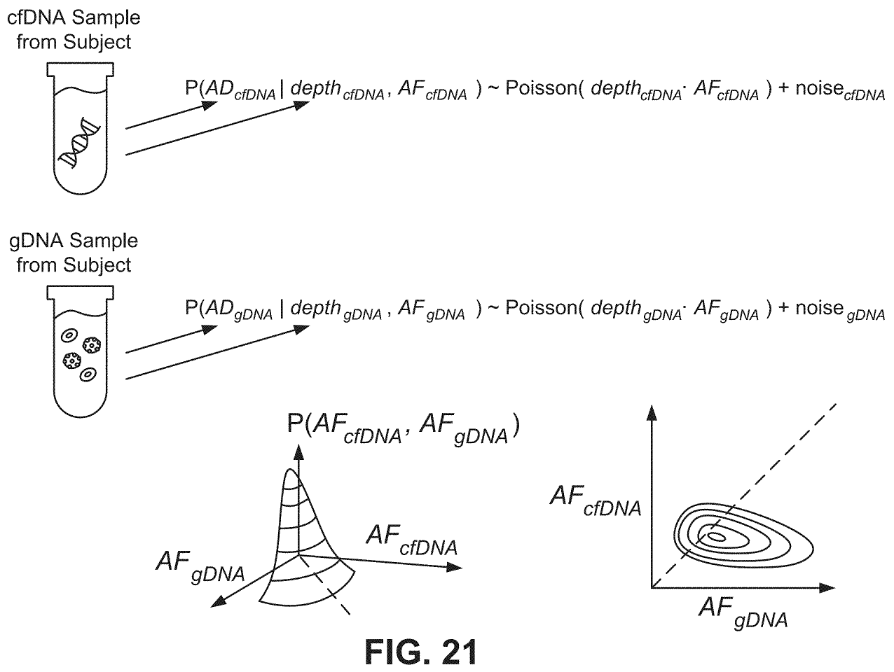

FIG. 20 is a flowchart of a method 2000 for using a joint model 1225 to process cell free nucleic acid (e.g., cfDNA) samples and genomic nucleic acid (e.g., gDNA) samples according to various embodiments. The joint model 1225 can be independent of positions of nucleic acids of cfDNA and gDNA. The method 2000 can be performed in conjunction with the methods 1800 and/or 1900 shown in FIGS. 18-19. For example, the methods 1800 and 1900 are performed to determine noise of nucleotide mutations with respect to cfDNA and gDNA samples of training data from health samples. FIG. 21 is a diagram of an application of a joint model according to one embodiment. Steps of the method 2000 are described below with reference to FIG. 21.

In step 2010, the sequence processor 1205 determines depths and ADs for the various positions of nucleic acids from the sequence reads obtained from a cfDNA sample of a subject. The cfDNA sample can be collected from a sample of blood plasma from the subject. Step 2010 can include previously described steps of the method disclosed in conjunction with FIG. 1.

In step 2020, the sequence processor 1205 determines depths and ADs for the various positions of nucleic acids from the sequence reads obtained from a gDNA of the same subject. The gDNA can be collected from white blood cells or a tumor biopsy from the subject. Step 1020 can include previously described steps of the method disclosed in conjunction with FIG. 1.

### VII. A. Example Signal of Joint Model

In step 2030, a joint model 1225 determines a likelihood of a “true” AF of the cfDNA sample of the subject by modeling the observed ADs for cfDNA. In one example, the joint model 1225 uses a Poisson distribution function, parameterized by the depths observed from the sequence reads of cfDNA and the true AF of the cfDNA sample, to model the probability of observing a given AD in cfDNA of the subject (also shown in FIG. 21). In some examples, the product of the depth and the true AF is the rate parameter of the Poisson distribution function, which represents the mean expected AF of cfDNA.

P(AD_{cfDNA}|depth_{cfDNA}, AF_{cfDNA})^{˜}Poisson(depth_{cfDNA}·AF_{cfDNA})+noise_{cfDNA}

The noise component noise_{cfDNA} is further described below in section VII. B. Example Noise of Joint Model. In some embodiments, other functions may be used to represent AD_{cfDNA}, examples of which include but are not limited to: negative binomial, Conway-Maxwell-Poisson distribution, zeta distribution, and zero-inflated Poisson.

In step 2040, the joint model 1225 determines a likelihood of a “true” AF of the gDNA sample of the subject by modeling the observed ADs fur gDNA. In one example, the joint model 1225 uses a Poisson distribution function parameterized by the depths observed from the sequence reads of gDNA and the true AF of the gDNA sample to model the probability of observing a given AD in gDNA of the subject (also shown in FIG. 21). The joint model 1225 can use a same function for modeling the likelihoods of true AF of gDNA and cfDNA, though the parameter values differ based on the values observed from the corresponding sample of the subject.

P(AD_{gDNA}|depth_{gDNA}, AF_{gDNA})^{˜}Poisson(depth_{gDNA}·AF_{gDNA})+noise_{gDNA}

The noise component noise_{gDNA} is further described below in section VII. B. Example Noise of Joint Model. In some embodiments, other functions can be used to represent AD_{gDNA}, examples of which include but are not limited to: negative binomial, Conway-Maxwell-Poisson distribution, zeta distribution, and zero-inflated Poisson.

Since the true AF of cfDNA, as well as the true AF of gDNA, are inherent properties of the biology of a particular subject, it may not necessarily be practical to determine an exact value of the true AF from either source. Moreover, various sources of noise also introduce uncertainty into the estimated values of the true AF. Accordingly, the joint model 1225 uses numerical approximation to determine the posterior distributions of true AF conditional on the observed data (e.g., depths and ADs) from a subject and corresponding noise parameters:

P(AF_{cfDNA}|depth_{cfDNA}, AD_{cfDNA}, \tilde{r}_{cfDNA}, mₚ_{_{cfDNA}})

P(AF_{gDNA}|depth_{gDNA}, AD_{gDNA}, \tilde{r}ₚ_{_{gDNA}}, mₚ_{_{gDNA}})

The joint model 1225 determines the posterior distributions using Bayes' theorem with a prior, for example, a uniform distribution. The priors used for cfDNA and gDNA may be the same (e.g., a uniform distribution ranging from 0 to 1) and independent from each other.

In an example, the joint model 1225 determines the posterior distribution of true AF of cfDNA using a likelihood function by varying the parameter, true AF of cfDNA, given a fixed set of observed data from the sample of cfDNA. Additionally, the joint model 1225 determines the posterior distribution of true AF of gDNA using another likelihood function by varying the parameter, true AF of gDNA, given a fixed set of observed data from the sample of gDNA. For both cfDNA and gDNA, the joint model 1225 numerically approximates the output posterior distribution by fitting a negative binomial (NB):

$$P ( AF \vert depth , AD ) ∝ {\sum }_{i = 0}^{AD} \frac{{{e}^{- AF \cdot depth} ( AF \cdot depth )}^{i}}{i !} \cdot NB ( AD - i , size = r , μ = m \cdot depth )$$

In an example, the joint model 1225 performs numerical approximation using the following parameters for the negative binomial, which can provide an improvement in computational speed:

P(AF|depth, AD)∝NB(AD, size=r,u=m·depth)

### Where:

$$\underline{m} = AF + m$$

$$\underline{r} = r \cdot \frac{{\underline{m}}^{2}}{{m}^{2 } }$$

Because the observed data is different between cfDNA and gDNA, the parameters determined for the negative binomial of cfDNA will vary from those determined for the negative binomial of gDNA.

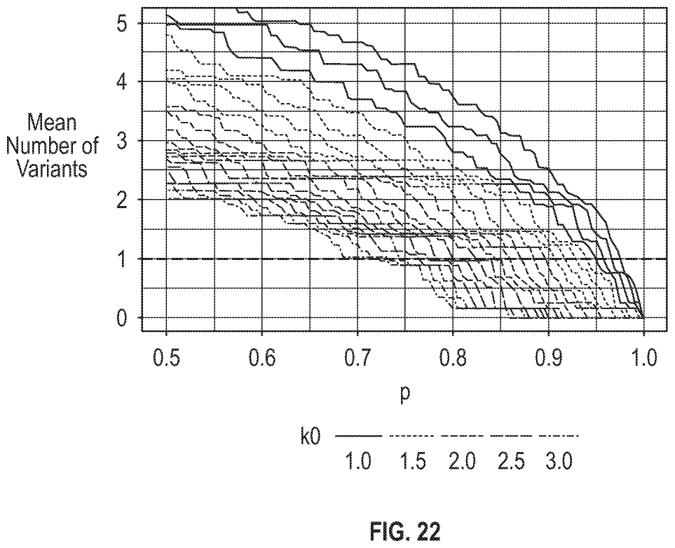

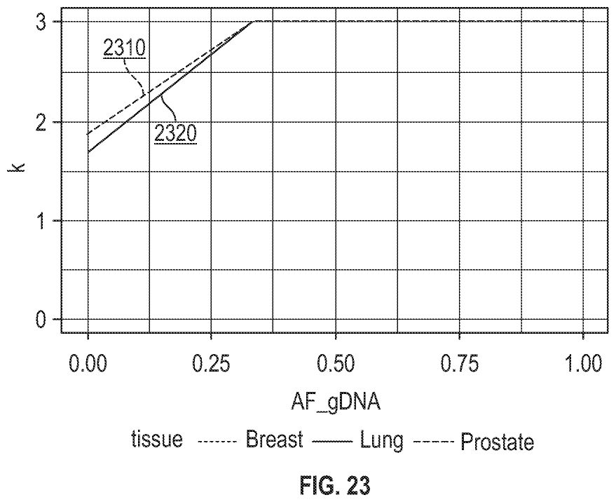

In step 2050, the variant caller 1240 determines, using the likelihoods, a probability that the true AF of the cfDNA sample is greater than a function of the true AF of the gDNA sample. The function can include one or more parameters, for example, empirically-determined k and p values stored in the parameter database 1230 and described with additional detail with reference to FIGS. 22-23. The probability represents a confidence level that at least some nucleotide mutations from the sequence reads of cfDNA are not found in sequence reads of reference tissue. The variant caller 1240 can provide this information to other processes for downstream analysis. For instance, a high probability indicates that nucleotide mutations from a subject's sequence reads of cfDNA and that are not found in sequence reads of gDNA may have originated from a tumor or other source of cancer within the subject. In contrast, low probability indicates that nucleotide mutations observed in cfDNA likely did not originate from potential cancer cells or other diseased cells of the subject. Instead, the nucleotide mutations may be attributed to naturally occurring mutations in healthy individuals, due to factors such as germline mutations, clonal hematopoiesis (unique mutations that form subpopulations of blood cell DNA), mosaicism, chemotherapy or mutagenic treatments, technical artifacts, among others.

In an embodiment, the variant caller 1240 determines that the posterior probability satisfies a chosen criteria based on the one or more parameters (e.g., k and p described below). The distributions of variants are conditionally independent given the sequences of the cfDNA and gDNA. That is, the variant caller 1240 presumes that ALTs and noise present in one of the ctDNA or gDNA sample are not influenced by those of the other sample, and vice versa. Thus, the variant caller 1240 considers the probabilities of the expected distributions of AD as independent events in determining the probability of observing both a certain true AF of cfDNA and a certain true AF of gDNA, given the observed data and noise parameters from both sources:

P(AF_{cfDNA}, AF_{gDNA}|depth, AD, \tilde{r}ₚ, mₚ)

P(AF_{cfDNA}|depth_{cfDNA}, AD_{cfDNA}, \tilde{r}ₚ_{_{cfDNA}}, mₚ_{_{cfDNA}})·P(AF_{gDNA}|depth_{gDNA}, AD_{gDNA}, \tilde{r}ₚ_{_{gDNA}}, mₚ_{_{gDNA}})

In the example 3D plot in FIG. 21, the probability P(AF_{cfDNA}, AF_{gDNA}) is plotted as a 3D contour for pairs of AF_{cfDNA} and AF_{gDNA} values. The example 2D slice of the 3D contour plot along the AF_{cfDNA} and AF_{gDNA} axis illustrates that the volume of the contour plot is skewed toward greater values of AF_{gDNA} relative to the values of AF_{cfDNA}. In other examples, the contour plot may be skewed differently or have a different form than the example shown in FIG. 21. To numerically approximate the joint likelihood, the sequence processor 1205 can calculate the volume defined by the 3D contour of P(AF_{cfDNA}, AF_{gDNA}) and a boundary line illustrated by the dotted line shown in the plots of FIG. 21. The sequence processor 1205 determines the slope of the boundary line according to the k parameter value, and the boundary line intersects the origin point. The k parameter value can account for a margin of error in the determined true AF. Particularly, the margin of error may cover naturally occurring mutations in healthy individuals such as germline mutations, clonal hematopoiesis, loss of heterozygosity (further described below with reference to FIG. 23), and other sources as described above. Since the 3D contour is split by the boundary line, at least a portion of variants detected from the cfDNA sample can potentially be attributed to variants detected from the gDNA sample, while another portion of the variants can potentially be attributed to a tumor or other source of cancer.

In an example, the sequence processor 1205 determines that a given criteria is satisfied by the posterior probability by determining the portion of the joint likelihood that satisfies the given criteria. The given criteria can be based on the k and p parameter, where p represents a threshold probability for comparison. For example, the sequence processor 1205 determines the posterior probability that true AF of cfDNA is greater than or equal to the true AF of gDNA multiplied by k, and whether the posterior probability is greater than p:

$$P ( {AF}_{cfDNA} \geq k \cdot {AF}_{gDNA} ) > p , where$$

$$P ( {AF}_{cfDNA} > {AF}_{gDNA} ) = {\int }_{0}^{1} {\int }_{k \cdot {AF}_{gDNA}}^{1} {f}_{cfDNA} ( {AF}_{cfDNA} ) \cdot {f}_{gDNA } ( {AF}_{gDNA} ) {dAF}_{cfDNA} {dAF}_{gDNA} = {\int }_{0}^{1} {f}_{gDNA} ( {AF}_{gDNA} ) [ {\int }_{k \cdot {AF}_{gDNA}}^{1} {f}_{cfDNA} ( {AF}_{cfDNA} ) \cdot {dAF}_{cfDNA} ] {dAF}_{gDNA} = {\int }_{0}^{1} {f}_{gDNA} ( {AF}_{gDNA} ) ( 1 - {F}_{cfDNA} ( k \cdot {AF}_{gDNA} ) ) {dAF}_{gDNA}$$

As shown in the above equations, the sequence processor 1205 determines a cumulative sum F_{cfDNA} of the likelihood of the true AF of cfDNA. Furthermore, the sequence processor 1205 integrates over the likelihood function of the true AF of gDNA. In another example, the sequence processor 1205 can determine the cumulative sum for the likelihood of the true AF of gDNA, and integrates over the likelihood function of the true AF of cfDNA. By calculating the cumulative sum of one of the two likelihoods (e.g., building a cumulative distribution function), instead of computing a double integral over both likelihoods for cfDNA and gDNA, the sequence processor 1205 reduces the computational resources (expressed in terms of compute time or other similar metrics) required to determine whether the joint likelihood satisfies the criteria and can also increase precision of the calculation of the posterior probability.

### VII. B. Example Noise of Joint Model

To account for noise in the estimated values of the true AF introduced by noise in the cfDNA and gDNA samples, the joint model 1225 can use other models of the processing system 1200 previously described with respect to FIGS. 14-19. In an example, the noise components shown in the above equations for P(AD_{cfDNA}|depth_{cfDNA}, AF_{cfDNA}) and P(AD_{gDNA}|depth_{gDNA}, AF_{gDNA}) are determined using Bayesian hierarchical models 1225, which may be specific to a candidate variant (e.g., SNV or indel). Moreover, the Bayesian hierarchical models 1225 can cover candidate variants over a range of specific positions of nucleotide mutations or indel lengths.

In one example, the joint model 1225 uses a function parameterized by cfDNA-specific parameters to determine a noise level for the true AF of cfDNA. The cfDNA-specific parameters can be derived using a Bayesian hierarchical model 1225 trained with a set of cfDNA samples, e.g., from healthy individuals. In addition, the joint model 1225 uses another function parameterized by gDNA-specific parameters to determine a noise level for the true AF of gDNA. The gDNA-specific parameters can be derived using another Bayesian hierarchical model 1225 trained with a set of gDNA samples, e.g., from the same healthy individuals. In some examples, the functions are negative binomial functions having a mean parameter in and dispersion parameter \tilde{r}, and can also depend on the observed depths of sequence reads from the training samples:

noise_{cfDNA}=NB(m_{cfDNA}·depth_{cfDNA}, \tilde{r}_{cfDNA})

noise_{gDNA}=NB(m_{gDNA}·depth_{gDNA}, \tilde{r}_{gDNA})

In other examples, the sequence processor 1225 can use a different type of function and types of parameters for cfDNA and/or gDNA. Because the cfDNA-specific parameters and gDNA-specific parameters are derived using different sets of training data, the parameters can be different from each other and particular to the respective type of nucleic acid sample. For instance, cfDNA samples can have greater variation in AF than gDNA samples, and thus \tilde{r}_{cfDNA} may be greater than \tilde{r}_{gDNA}.

### VIII. EXAMPLES FOR JOINT MODELS

The example results shown in the following figures were determined by the processing system 1200 using one or more trained models 1225, which can include joint models and Bayesian hierarchical models. For purposes of comparison, some example results were determined using an empirical threshold or a simple model and are referred to as “empirical threshold” examples and denoted as “threshold” in the figures; these example results were not obtained using one of the trained models 1225. In various embodiments, the results were generated using one of a number of example targeted sequencing assays, including “targeted sequencing assay A” and “targeted sequencing assay B,” also referred to herein and in the figures as “Assay A” and “Assay B,” respectively.

In an example process performed for a targeted sequencing assay, two tubes of whole blood were drawn into Streck BCTs from healthy individuals (self-reported as no cancer diagnosis). After plasma was separated from the whole blood, it was stored at −80° C. Upon assay processing, cfDNA was extracted and pooled from two tubes of plasma. Corielle genomic DNA (gDNA) were fragmented to a mean size of 180 base pairs (bp) and then sized selected to a tighter distribution using magnetic beads. The library preparation protocol was optimized for low input cell free DNA (cfDNA) and sheared gDNA. Unique molecular identifiers (UMIs) were incorporated into the DNA molecules during adapter ligation. Flowcell clustering adapter sequences and dual sample indices were then incorporated at library preparation amplification with PCR. Libraries were enriched using a targeted capture panel.

Target DNA molecules were first captured using biotinylated single-stranded DNA hybridization probes and then enriched using magnetic streptavidin beads. Non-target molecules were removed using subsequent wash steps. The HiSeq X Reagent Kit v2.5 (Illumina; San Diego, Calif.) was used for flowcell clustering and sequencing. Four libraries per flowcell were multiplexed. Dual indexing primer mix was included to enable dual sample indexing reads. The read lengths were set to 150, 150, 8, and 8, respectively for read 1, read 2, index read 1, and index read 2. The first 6 base reads in read 1 and read 2 are the UMI sequences.

### VIII. A. Example Parameters for Joint Model

FIG. 22 is a diagram of observed counts of variants in samples from healthy individuals according to one embodiment. Each data point corresponds to a position (across a range of nucleic acid positions) of a given one of the individuals. The parameters k and p used by the joint model 1225 for joint likelihood computations can be selected empirically (e.g., to tune sensitivity thresholds) by cross-validating with sets of cfDNA and gDNA samples from healthy individuals and/or samples known to have cancer. The example results shown in FIG. 22 were obtained with Assay B and using blood plasma samples for the cfDNA and white blood cell samples for the gDNA. For given parameter values for k (“k0” as shown in FIG. 22) and p, the diagram plots a mean number of variants, which represents a computed upper confidence bound (UCB) of false positives for the corresponding sample. The diagram indicates that the number of false positives decreases as the value of p increases. In addition, the plotted curves have greater numbers of false positives for lower values of k, e.g., closer to 1.0. The dotted line indicates a target of one variant, though the empirical results show that the mean number of false positives mostly fall within the range of 1-5 variants, fork values between 1.0 and 5.0, and p values between 0.5 and 1.0.

The selection of parameters can involve a tradeoff between a target sensitivity (e.g., adjusted using k and p) and target error (e.g., the upper confidence bound). For given pairs of k and p values, the corresponding mean number of false positives may be similar in value, though the sensitivity values may exhibit greater variance. In some embodiments, the sensitivity is measured using percent positive agreement (PPA) values for tumor, in contrast to PPA for cfDNA, which can be used as a measurement of specificity:

$${PPA}_{tumor} = \frac{tumor + cfDNA}{tumor}$$

$${PPA}_{cfDNA} = \frac{tumor + cfDNA}{cfDNA}$$

In the above equations, “tumor” represents the number of mean variant calls from a ctDNA sample using a set of parameters, and “cfDNA” represents the number of mean variant calls from the corresponding ctDNA sample using the same set of parameters.

In some embodiments, cross-validation is performed to estimate the expected fit of the joint model 1225 to sequence reads (for a given type of tissue) that are different from the sequence reads used to train the joint model 1225. For example, the sequence reads can be obtained from tissues having lung, prostate, and breast cancer, etc. To avoid or reduce the extent of overfitting the joint model 1225 for any given type of cancer tissue, parameter values derived using samples of a set of types of cancer tissue are used to assess statistical results of other samples known to have a different type of cancer tissue. For instance, parameter values for lung and prostate cancer tissue are applied to a sample having breast cancer tissue. In some embodiments, one or more lowest k values from the lung and prostate cancer tissue data that maximizes the sensitivity is selected to be applied to the breast cancer sample. Parameter values can also be selected using other constraints such as a threshold deviation from a target mean number of false positives, or 95% UCB of at most 3 per sample. The processing system 1200 can cycle through multiple types of tissue to cross validate sets of cancer-specific parameters.

FIG. 23 is a diagram of example parameters for a joint model 1225 according to various embodiments. The parameter values for k can be determined as a function of AF observed in gDNA samples, and can vary based on a particular type of cancer tissue, breast, lung, or prostate as illustrated. The curve 2310 represents parameter values for breast and prostate cancer tissue, and the curve 2320 represents parameters values for lung cancer tissue. Although the examples thus far describe k and p generally and with reference to implementations where these parameters are fixed, in practice k and p may vary as any function of AF observed in the gDNA sample. In the example shown in the FIG. 23, the function is a hinge loss function with a hinge value (or lower threshold value), e.g., one-third. Specifically, the function specifies that k equal a predetermined upper threshold, e.g., 3, for AF_{gDNA} values greater than or equal to the hinge value. For AF_{gDNA} values less than the hinge value, the corresponding k values modulates with AF_{gDNA}. The example of FIG. 23 specifically illustrates that the k values for AF_{gDNA} values less than one-third may be proportional to AF_{gDNA} according to a coefficient (e.g., slope in the case of a linear relationship), which may vary between types of cancer tissue. In other embodiments, the joint model 1225 can use another type of loss function such as square loss, logistic loss, cross entropy loss, etc.

The joint model 1225 can alter k according to a hinge loss function or another function to guard against non-tumor or disease related effects where a fixed value for k would not accurately capture and categorize those events. The hinge loss function example is particularly targeted at handling loss of heterozygosity (LOH) events. LOH events are germline mutations that occur when a copy of a gene is lost from one of an individual's parents. LOH events may contribute to significant portions of observed AF of a gDNA sample. By capping the k values to the predetermined upper threshold of the hinge loss function, the joint model 1225 can achieve greater sensitivity for detecting true positives in most sequence reads while also controlling the number of false positives that would otherwise be flagged as true positives due to the presence of LOH. In other embodiments, k and p can be selected based on training data specific to a given application of interest, e.g., having a target population or sequencing assay.

In some embodiments, the joint model 1225 takes into account both the AF of a gDNA sample and a quality score of the gDNA sample to guard against underweighting low AF candidate variants. As previously described with reference to FIGS. 13, 14, and 19, the quality score generated by the score engine 1235 for a noise model can be used to estimate the probability of error on a Phred scale. Additionally, the joint model 1225 can use a modified piecewise function for the hinge function. For example, the piecewise function includes two or more additive components. One component is a linear function based on the Al of the gDNA sample, and another component is an exponential function based on the quality score of the gDNA sample. Given a quality score threshold and a maximum AF scaling factor kₘₐₓ, the joint model 1225 determines, using the exponential component of the piecewise function:

$${k}_{ma x} \cdot P ( not error ) = \frac{1 - P ( error )}{{P ( error )}_{m i n}}$$

In the above calculation, P(not error) is the probability that an allele of the gDNA sample is not an error, P(error) is the probability that the allele of the gDNA sample is an error, and P(error)ₘᵢₙ is a minimum probability of error. A minimum threshold for error rate can be empirically determined as the intersecting point for the quality score densities between the likely somatic and likely germline candidate variants of alleles of the gDNA sample.

### VIII. B. Example Detected Genes Using Joint Model

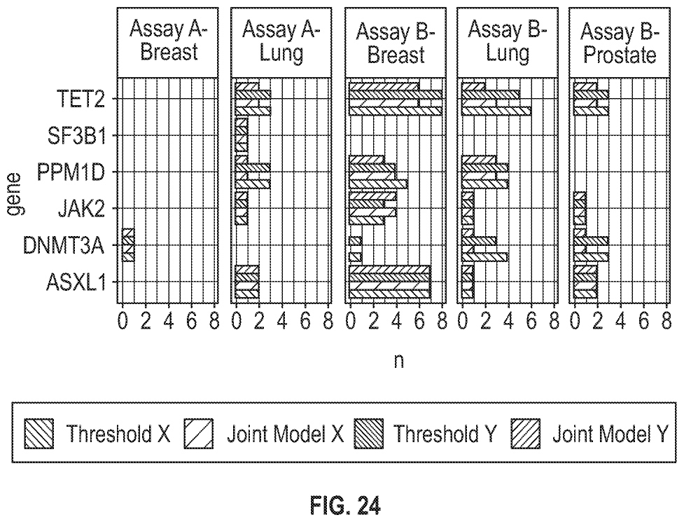

FIG. 24 is a diagram of a set of genes detected from targeted sequencing assays using a joint model 1225 according to various embodiments. The set includes genes that are commonly mutated during clonal hematopoiesis. The sequence processor 1205 determines the results using assays A and B and samples known to have breast, lung, and prostate cancer. The tests “Threshold X” and “Joint Model X” do not include nonsynonymous mutations, while the tests “Threshold Y” and “Joint Model Y” do include nonsynonymous mutations. The example results obtained using the joint model 1225 reduces the counts (denoted as “n” on the x-axis as shown in FIGS. 24-24) of detected germline mutations from samples of various types of tissue, in comparison to the counts detected using an empirical threshold. For instance, as illustrated by the graph for assay B with lung cancer, “Threshold X” and “Threshold Y” results in counts of 5 and 6 detected TET2 genes, respectively. The “Joint Model X” and “Joint Model Y” results in counts of 2 and 3 detected TET2 genes, respectively, which indicates that the joint model 1225 provides improved sensitivity.

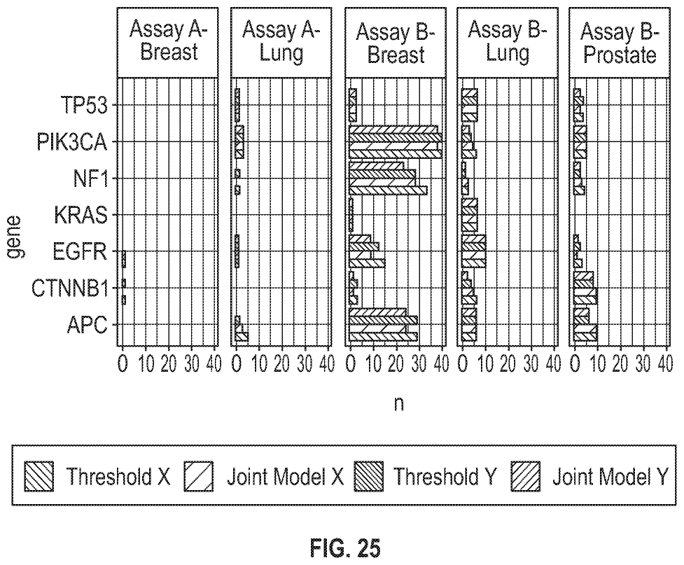

FIG. 25 is a diagram of another set of genes detected from targeted sequencing assays using a joint model 1225 according to some embodiments. The example results indicate that the sensitivity for detecting driver genes of the joint model 1225 is comparable to that of filters that do not use a model. That is, the joint model 1225 does not significantly over filter the detected driver genes relative to the results obtained using an empirical threshold.

### IX. EXAMPLE TUNING OF JOINT MODEL

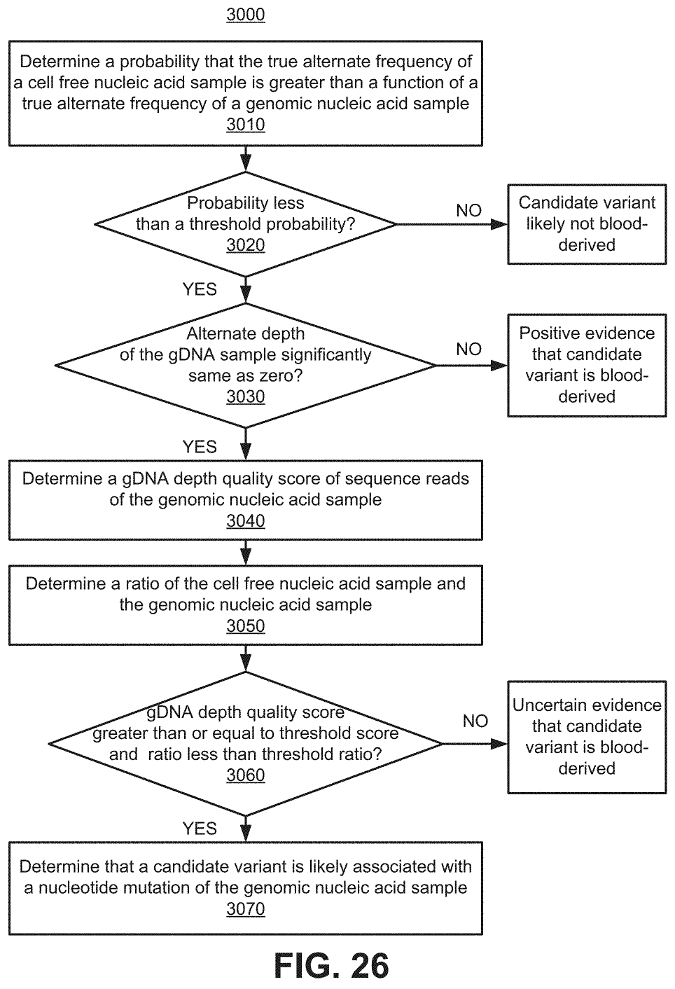

FIG. 26 is a flowchart of a method 3000 for tuning a joint model 1225 to process cell free nucleic acid (e.g., cfDNA) samples and genomic nucleic acid (e.g., gDNA) samples according to some embodiments. The method 3000 can be performed in conjunction with the methods 1800, 1900, and/or 2000 shown in FIGS. 18-20, or another similar method. For example, the method 3000 is performed using a joint model 1255 to determine the probability for step 3010 of the method 3000. The examples described with respect to FIGS. 26-28 reference blood (e.g., white blood cells) of a subject as the source of the gDNA sample, though it should be noted that in other examples, the gDNA can be from a different type of biological sample. The processing system 1200 can implement at least a portion of the method 3000 as a decision tree to filter or process candidate variants in the cfDNA sample. For instance, the processing system 1200 determines whether a candidate variant is likely associated or not with the gDNA sample, or if an association is uncertain. An association can indicate that the variant can be accounted for by a mutation in the gDNA sample (e.g., due to factors such as germline mutations, clonal hematopoiesis, artifacts, edge variants, human leukocyte antigens such as HLA-A, etc.) and thus likely not tumor-derived and not indicative of cancer or disease. The method 3000 can include different or additional steps than those described in conjunction with FIG. 26 in some embodiments or perform steps in different orders than the order described in conjunction with FIG. 26.

### IX. A. Example Quality Score and Ratio of Joint Model

Referring still to FIG. 26, in step 3010, the sequence processor 1205 determines a probability that the true alternate frequency of a cfDNA sample is greater than a function of a true alternate frequency of a gDNA sample. In some examples, step 3010 corresponds to previously described step 2050 of the method 2000 shown in FIG. 20.

In step 3020, the sequence processor 1205 determines whether the probability is less than a threshold probability. As an example, the threshold probability may be 0.8, however in practice the threshold probability can be any value between 0.5 and 0.999 (e.g., determined based on a desired filtering stringency), static or dynamic, vary by gene and/or set by position, or other macro factors, etc. Responsive to determining that the probability is greater than or equal to the threshold probability, the sequence processor 1205 determines that the candidate variant is likely not associated with the gDNA sample such as a blood draw including white blood cells of a subject, i.e., not blood-derived. For example, the candidate variant is typically not present in sequence reads of the gDNA sample for a healthy individual. Accordingly, the variant caller 240 can call the candidate variant as a true positive that potentially associated with cancer or disease, e.g., potentially tumor-derived.

In step 3030, the sequence processor 1205 determines whether the alternate depth of the gDNA sample is significantly the same as or different than zero. For instance, the sequence processor 1205 performs an assessment using a quality score of the candidate variant determined by the score engine 1235 using a noise model 1225 as previously described with reference to FIGS. 13, 14, and 19. The sequence processor 1205 can also compare the alternate depth against a threshold depth, e.g., determining whether the alternate depth is less than or equal to a threshold depth. As an example, the threshold depth can be 0 or 1 reads. Responsive to determining that the alternate depth of the gDNA sample is significantly different than zero, the sequence processor 1205 determines that there is positive evidence that the candidate variant is associated with nucleotide mutations not caused by cancer or disease. For instance, the candidate variant is blood-derived based on mutations that may typically occur in sequence reads of healthy white blood cells.

Responsive to determining that the alternate depth of the gDNA sample is not significantly nonzero, the sequence processor 1205 determines that the candidate variant is possibly associated with the gDNA sample, but does not make a determination of a source of the candidate variant without further checking by the score engine 1235 as described below. In other words, the sequence processor 1205 may be uncertain about whether the candidate variant is blood-derived or tumor-derived. In some embodiments, the sequence processor 1205 can select one of multiple threshold depths for comparison with the alternate depth. The selection can be based on a type of processed sample, noise level, confidence level, or other factors.

In step 3040, the score engine 1235 determines a gDNA depth quality score of sequence reads of the gDNA sample. In some embodiments, the score engine 1235 calculates the gDNA depth quality score using an alternate depth of the gDNA sample, where C is a predetermined constant (e.g., 2) to smooth the gDNA depth quality score using a weak prior, which avoids divide-by-zero computations:

$${AD}_{gDNA} depth quality score = \frac{{AD}_{gDNA}}{\sqrt{{AD}_{gDNA} + C}}$$

In step 3050, the score engine 1235 determines a ratio of sequence reads of the gDNA sample. The ratio can represent the observed cfDNA frequency and observed gDNA frequency in the processed samples. In an example, the score engine 1235 calculates the ratio using the depths and alternate depths of the cfDNA sample and gDNA sample:

$$ratio = \frac{( {AD}_{cfDNA} + {C}_{1} ) ( {depth}_{gDNA} + {C}_{2} )}{( {AD}_{gDNA} + {C}_{3} ) ( {depth}_{cfDNA} + {C}_{4} )}$$

The score engine 1235 can use the predetermined constants C₁, C₂, C₃, and C₄ to smooth the ratio by a weak prior. As examples, the constants can be: C₁=2, C₂=4, C₃=2, and C₄=4. Thus, the score engine 1235 can avoid a divide-by-zero computation if one of the depths or alternate depths in the ratio denominator equals zero. Thus, the score engine 1235 can use the predetermined constants to steer the ratio to a certain value, e.g., 1 or 0.5.

In step 3060, the sequence processor 1205 determines whether the gDNA depth quality score is greater than or equal to a threshold score (e.g., 1) and whether the ratio is less than a threshold ratio (e.g., 6). Responsive to determining that the gDNA depth quality score is less than the threshold score or that the ratio is greater than or equal to the threshold ratio, the sequence processor 1205 determines that there is uncertain evidence regarding association of the candidate variant with the gDNA sample. Stated differently, the sequence processor 1205 can be uncertain about whether the candidate variant is blood-derived or tumor-derived because the candidate variant appears “bloodish” but there is no definitive evidence that a corresponding mutation is found in healthy blood cells.

In step 3070, responsive to determining that the gDNA depth quality score is greater than or equal to the threshold score and that the ratio is less than the threshold ratio, the sequence processor 1205 determines that the candidate variant is likely associated with a nucleotide mutation of the gDNA sample. In other words, the sequence processor 1205 determines that although there is not definitive evidence that a corresponding mutation is found in healthy blood cells, the candidate variant appears “bloodier” than normal. [“bloodier”/likely]

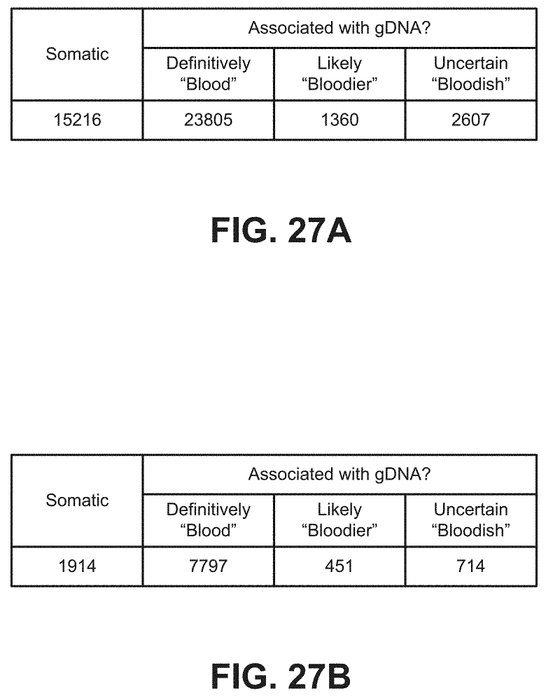

Thus, the sequence processor 1205 can use the ratio and gDNA depth quality score to tune the joint model 1225 to provide greater granularity in determining whether certain candidate variants should be filtered out as false positives (e.g., initially predicted as tumor-derived, but actually blood-derived), true positives, or uncertain due to insufficient evidence or confidence to classify into either category. For example, based on the result of the method 3000, the sequence processor 1205 can modify one or more of the parameters (e.g., k parameter) for a hinge loss function of the joint model 1225. In some embodiments, the sequence processor 1205 uses one or more steps of the method 3000 to assign candidate variants to different categories, for instance, “definitively,” “likely,” or “uncertain” association with gDNA (e.g., as shown in FIGS. 27A-43).

### IX. B. Example Decision Tree

In various embodiments, the processing system 1200 processes candidate variants using one or more filters in addition to the steps described with reference to the flowchart of the method 3000 shown in FIG. 26. The sequence processor 1205 can implement the filters in a sequence as part of a decision tree, where the sequence processor 1205 continues to check criteria of the filters until a given candidate variant “exits” the decision tree, e.g., because the given candidate variant is filtered upon satisfying at least one of the criteria. A filtered candidate variant may indicate that the candidate variant can be accounted for by a source or cause of mutations naturally occurring in healthy individuals (e.g., associated with white blood cell gDNA) or due to process errors.

In some embodiments, the sequence processor 1205 filters candidate variants of sequence reads of a ctDNA sample responsive to determining that there is no quality score for the sequence reads. The score engine 1235 can determine quality scores for candidate variants using a noise model 1225 as previously described with reference to FIGS. 13, 14, and 19. The score engine 1235 can determine the quality scores with no base alignment. In some examples, the quality score may be missing for some samples or candidate variants due to a lack of training data for the joint model 1225 or poor training data that fails to produce useful parameters for a given candidate variant. For instance, high noise levels in sequence reads may lead to unavailability of useful training data. The score engine 1235 can tune specificity and selectivity of the joint model 1225 based on whether a single variant is processed or if the sequence processor 1205 is controlling for a targeted panel. As other examples, the sequence processor 1205 filters a candidate variant responsive to determining that the candidate variant is an edge variant artifact, has less than a threshold cfDNA depth (e.g., 1200 sequence reads), has less than a threshold cfDNA quality score (e.g., 60), or corresponds to human leukocyte antigens (HLA), e.g., HLA-A. Since sequences associated with HLA-A may be difficult to align, the sequence processor 1205 can perform a custom filtering or variant calling process for sequences in these regions.

In some embodiments, the sequence processor 1205 filters candidate variants determined to be associated with germline mutations. The sequence processor 1205 can determine that a candidate variant is germline by determining that the candidate variant occurs at an appropriate frequency corresponding to a given germline mutation event and is present at a particular one or more positions (e.g., in a nucleotide sequence) known to be associated with germline events. Additionally, the sequence processor 1205 can determine a point estimate of gDNA frequency, where C is a constant (e.g., 0.5):

$${point}_{afDNA} = \frac{{AD}_{gDNA}}{{depth}_{gDNA} + C}$$

The sequence processor 1205 can determine that a candidate variant is germline responsive to determining that point_{afDNA} is greater than a threshold point estimate threshold (e.g., 0.3). In some embodiments, the sequence processor 1205 filters candidate variants responsive to determining that a number of variants associated with local sequence repetitions is greater than a threshold value. For example, an “AAAAAA” or “ATATATAT” local sequence repetition may be the result of a polymerase slip that causes an increase in local error rates.

### IX. C. Examples for Tuned Joint Model

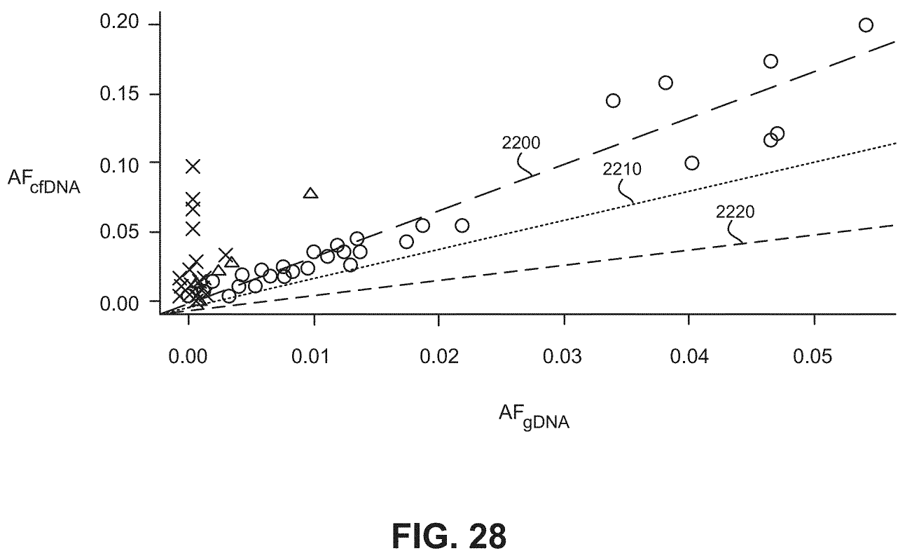

FIG. 27A is a table of example counts of candidate variants of cfDNA samples according to various embodiments. The example data in FIGS. 27A-B and FIG. 28 were generated using sequence reads obtained from a sample set of individuals of the CCGA study described herein. The cfDNA samples include samples from individuals known to have cancer or another type of disease. In the example shown in FIG. 27A, the processing system 1200 uses the method 3000 of FIG. 26 to determine that 23805 of the candidate variants are “definitively” associated with gDNA (e.g., accounted for by germline mutations or clonal hematopoiesis in blood) and that 1360 of the candidate variants are “likely” associated with gDNA (e.g., “bloodier” or greater than a threshold confidence level). Thus, the processing system 1200 can filter out these candidate variants from the joint model 1225 or another pipeline, e.g., such that these candidate variants are classified as blood-derived. The processing system 1200 can determine to neither categorize the count of 2607 “uncertain” (e.g., “bloodish”) candidate variants as tumor-derived nor blood-derived. Thus, by tuning the joint model 1225, for example, using the gDNA ratio and gDNA depth quality score from the method 3000, the processing system 1200 improves granularity (e.g., different levels of confidence) in classifying sources of candidate variants. FIG. 27B is a table of example counts of candidate variants of cfDNA samples from healthy individuals according to an embodiment. The example counts shown in FIGS. 27A-B were determined by the processing system 1200 using a threshold depth of 1200 reads, threshold quality score of 60 (e.g., on a Phred scale), quality score at the corresponding position having a mean squared deviation from germline mutation frequency threshold of 0.005, threshold point estimate of gDNA frequency of 0.3, threshold artifact recurrence rate of 0.05, threshold local sequence repetition count of 7, threshold probability (e.g., that the true alternate frequency of a ctDNA sample is greater than a function of a true alternate frequency of a gDNA sample) of 0.8, threshold gDNA depth of 0, threshold gDNA depth quality score of 1, and threshold gDNA sample ratio of 6. Furthermore, the processing system 1200 filtered out candidate variants with no quality score, somatic variants, and HLA-A regions.

FIG. 28 is a diagram of candidate variants plotted based on ratio of cfDNA and gDNA according to one embodiment. For each of a number of plotted candidate variants of a subject, the x-axis value represents the AF observed in gDNA samples and the y-axis represents the AF observed in a corresponding cfDNA sample of the subject. The example shown in FIG. 28 includes candidate variants passed by a joint model 1225 using a hinge function such as the curve 2310 or curve 2320 illustrated in FIG. 23. For this example data and the recited parameters above, the processing system 1200 determines that the cluster of candidate variants depicted as cross marks toward the left of the plot, which have a relatively higher AF_{cfDNA} to AF _{gDNA} ratio, are likely to be not associated with nucleotide mutations naturally occurring in white blood cells, and thus predicted as tumor-derived. The dotted line 2220 is a reference line representing a 1:1 AF_{cfDNA} to AF_{gDNA} ratio. A hinge function is represented by the dotted graphic 2210, which may not necessarily be a line (e.g., may include multiple segments connected at one or more hinges). The cluster of candidate variants depicted as circles have relatively lower AF_{cfDNA} to AF_{gDNA} ratios, but were still passed by the joint model 1225 when using the hinge function represented by 2210 (e.g., because several of the candidate variants are plotted above 3210. However, some of these candidate variants may actually be associated with gDNA, e.g., blood-derived, and should be filtered Out instead of being called as tumor-derived. The dotted line 2200 is a regression line determined using robust fit regression on the clusters of data points depicted in the cross marks. By tuning the hinge function using the regression line 2200, the joint model 1225 can filter out more of the candidate variants that may actually be blood-derived. In some embodiments, 2200, 2210, and 2220 each intersect the origin (0, 0). The processing system 1200 determines that there is uncertain evidence as to whether the cluster of candidate variants depicted as triangles (located generally between the clusters of cross marks and circle-type candidate variants) are blood or tumor-derived.

To improve the accuracy of catching these candidate variants, the processing system 1200 can use the filters as described above with reference to FIG. 26. Further, the processing system 1200 can tune the joint model 1225 by using more aggressive parameters for a hinge function under certain conditions. For example, the processing system 1200 uses a greater probability threshold (e.g., for step 3020 of the method 3000 shown in FIG. 26) responsive to determining that the AD of the gDNA sample is greater than a threshold depth (e.g., 0), which is supportive evidence of nucleotide mutations in blood of healthy samples. In some embodiments, the processing system 1200 determines a modified hinge function (or another type of function for classifying true and false positives) using the greater probability threshold. For instance, the modified function can have a sharper cutoff (e.g., relative to the curves 2310 and 2320 of FIG. 23) that would filter out at least some candidate variants of the cluster along the dotted diagonal lines in FIG. 28. The processing system 1200 can also tune the modified function using the gDNA sample quality score or ratio as determined in steps 3040 and 3050 of the method 3000, respectively.

### X. EXAMPLE EDGE FILTERING

X. A. Example Training Distributions of Features from Artifact and Non-Edge Variants

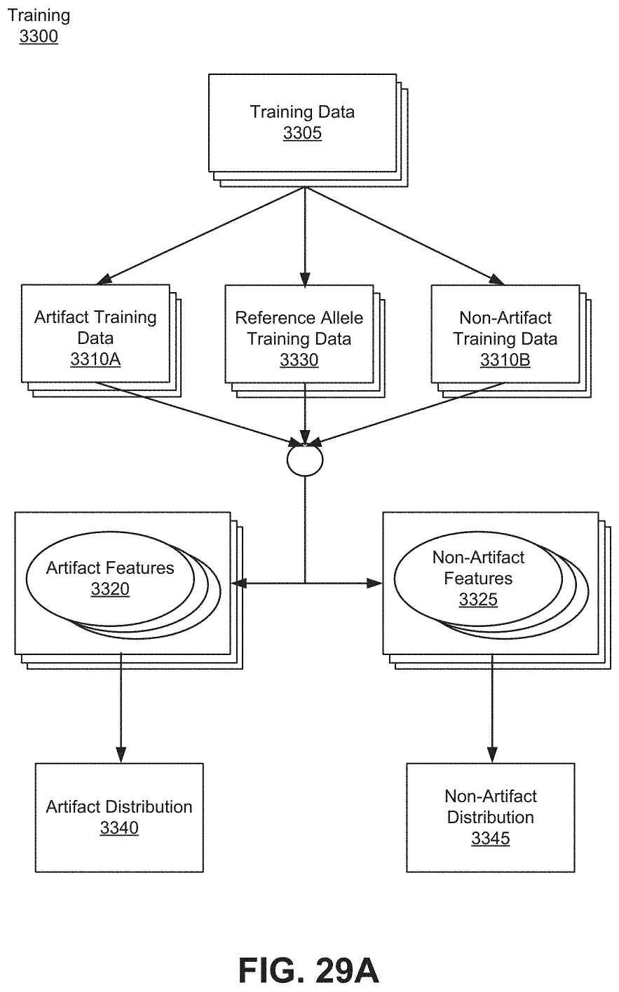

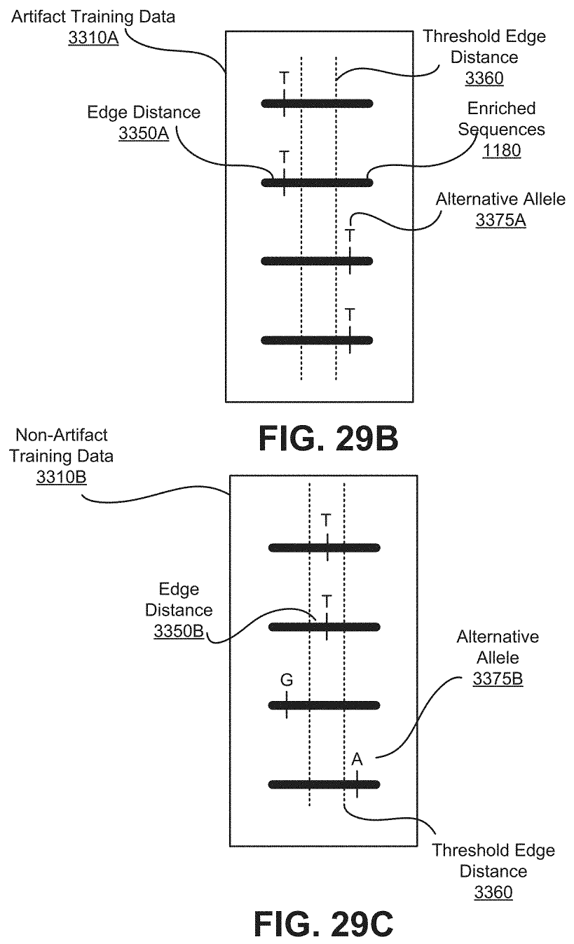

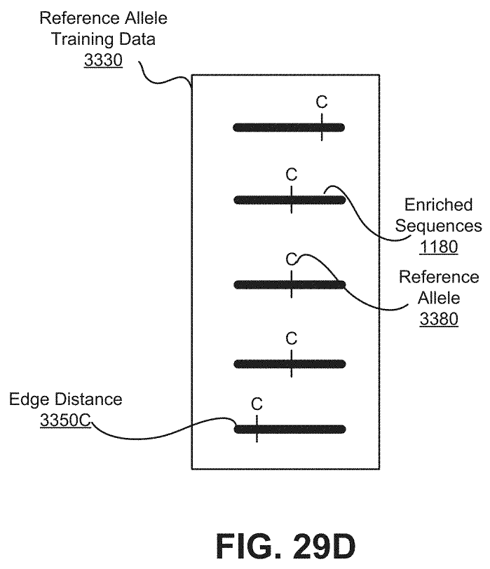

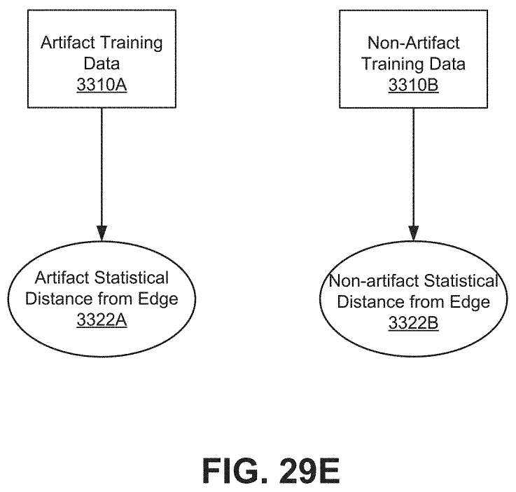

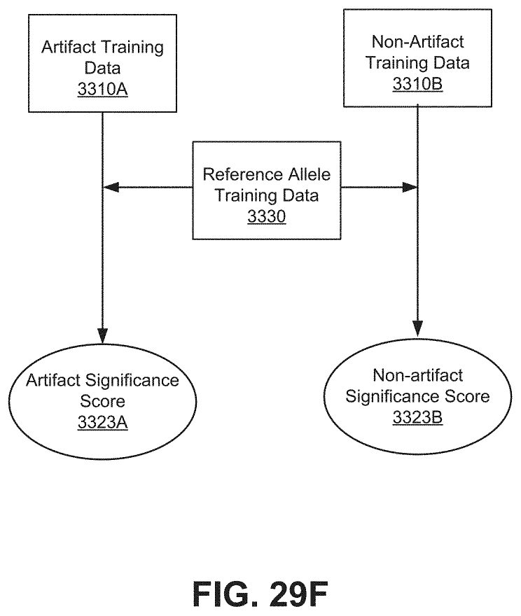

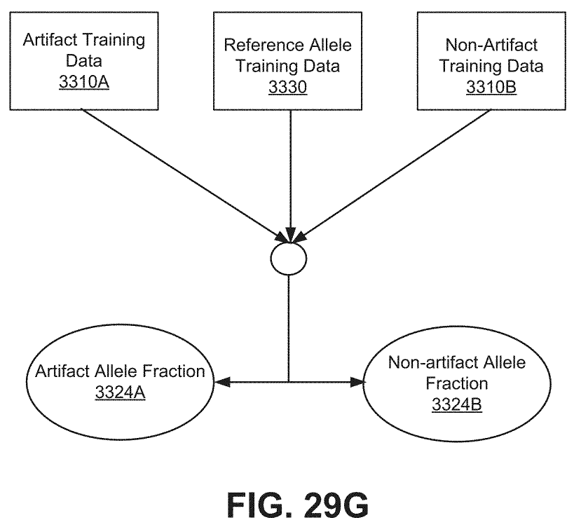

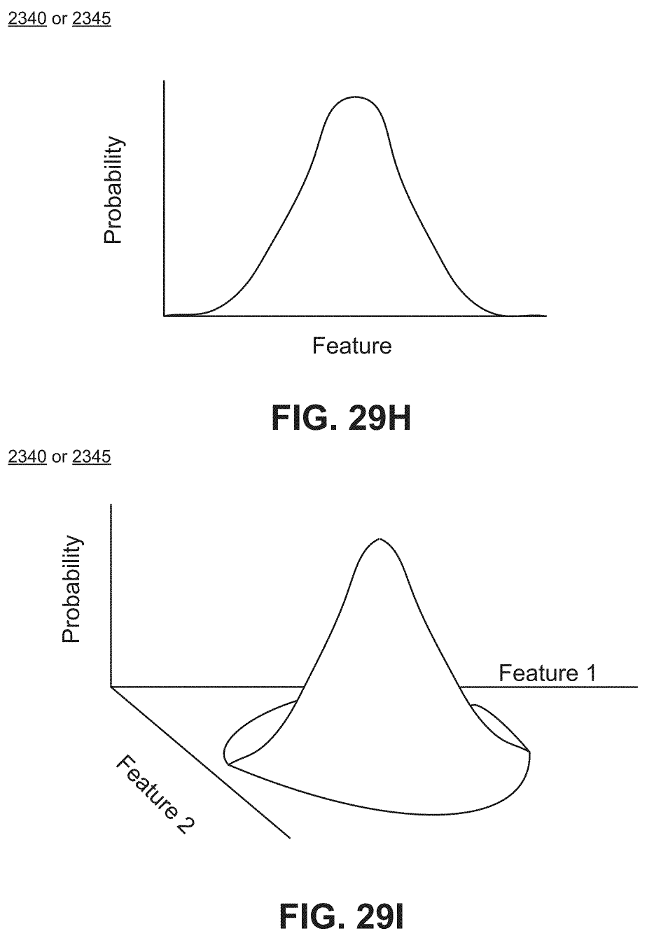

FIG. 29A depicts a process of generating an artifact distribution and a non-artifact distribution using training variants according to one embodiment. The edge filter 1250 generates artifact distribution 3340 and non-artifact distribution 3345 during a training process 3300 using training data 3305 from previous samples (e.g., training samples). Once generated, the artifact distribution 3340 and non-artifact distribution 3345 can each be stored (e.g., in the model database 1215) for subsequent retrieval at a needed time.

Training data 3305 includes various sequence reads, such as sequence reads sequenced from enriched sequences 1180 (see, e.g., FIG. 29B). Sequence reads in the training data 3305 can correspond to various positions on the genome. In various embodiments, sequence reads in the training data 3305 are obtained from more than one training sample.

The edge filter 1250 categorizes sequence reads in the training data 3305 into one of an artifact training data 3310A category, reference allele training data 3330 category, or non-artifact training data 3310B category. In various embodiments, sequence reads in the training data 3305 can also be categorized into a no result or a no classification category responsive to determining that the sequence reads do not satisfy the criteria to be placed in any of the artifact training data 3310A category, reference allele training data 3330 category, or non-artifact training data 3310B category.

As shown in FIG. 29A, there may be multiply groups of artifact training data 3310A, multiple groups of reference allele training data 3330, and multiple groups of non-artifact training data 3310B. Generally, sequence reads that are in a group cross over (overlap) a common position in the genome. In various embodiments, sequence reads in a group derive from a single training sample (e.g., a training sample obtained from a single individual) and cross over the common position in the genome. For example, given sequence reads from M different training samples obtained from M different individuals, there can be M different groups each including sequence reads from one of the M different training samples. Although the subsequent description refers to groups of sequence reads that cross over a common position on the genome, the description can be further expanded to other groups of sequence reads that cross over other positions on the genome.

Sequence reads that correspond to a common position on the genome include: 1) sequencing reads that include a nucleotide base at the position that is different from the reference allele (e.g., an ALT) and 2) sequencing reads that include a nucleotide base at the position that matches the reference allele. A sequence read can be sequenced from an enriched sequence 1180 that includes an ALT (e.g., the thymine in enriched sequence 1180A or 1180C) or can include the reference allele (e.g., the cytosine in enriched sequence 1180B).

The edge filter 1250 categorizes sequence reads that include an ALT into one of the artifact training data 3310A or non-artifact training data 3310B. Specifically, sequence reads that satisfy one or more criteria are categorized as artifact training data 3310A. The criteria can be a combination of a type of mutation of the ALT and a location of the ALT on the sequence read. Referring to an example of a type of mutation, sequence reads categorized as artifact training data include an alternative allele that is either a cytosine to thymine (C>T) nucleotide base substitution or a guanine to adenine (G>A) nucleotide base substitution. Referring to an example of the location of the alternative allele, the alternative allele is less than a threshold number of base pairs from an edge of a sequence read. In one implementation, the threshold number of base pairs is 25 nucleotide base pairs, however, the threshold number may vary by implementation.

FIG. 29B depicts sequence reads that are categorized in an artifact training data 3310A category according to one embodiment. Additionally, each of the sequence reads satisfy one or more criteria. For example, each sequence read includes an alternative allele 3375A that is a C>T nucleotide base substitution. Additionally, the alternative allele 3375A on each sequence read is located at an edge distance 3350A that is less than a threshold edge distance 3360.

Sequence reads with an alternative allele that are categorized into the non-artifact training data 3310B category are all other sequence reads with an alternative allele that do not satisfy the criteria of being categorized as artifact training data 3310A. For example, any sequence read that includes an alternative allele that is not one of a C>T or G>A nucleotide base substitution is categorized as a non-edge training variant. As another example, irrespective of the type of nucleotide mutation, any sequence read that includes an alternative allele that is located greater than a threshold number of base pairs from an edge of a sequence read is categorized as non-artifact training data 3310B. In one implementation, the threshold number of base pairs is 25 nucleotide base pairs, however, the threshold number may vary by implementation.

FIG. 29C depicts sequence reads that are categorized in the non-artifact training data 3310B category according to one embodiment. Here, each of the sequence reads includes an alternative allele 3375B that does not satisfy both criteria. For example, each alternative allele 3375B can either be a non C>T or non G>A nucleotide base substitution, irrespective of the location of the alternative allele 3375B. As another example, each alternative allele 3375B is a C>T or G>A nucleotide base substitution, but is located with an edge distance 3350B that is greater than the threshold edge distance 3360.

Referring now to the reference allele training data 3330 category, sequence reads that include the reference allele are categorized in the reference allele training data 3330 category. FIG. 29D depicts sequence reads corresponding to the same position in the genome that are categorized in the reference allele training data 3330 category according to one embodiment. As an example, the sequence reads shown in FIG. 29D each include the reference allele 3380. Additionally, these sequence reads that include the reference allele 3380 are categorized in the reference allele training data 3330 irrespective of the edge distance 3350C between the reference allele and the edge of the sequence read.

Returning to FIG. 29A, the edge filter 1250 extracts features from groups of sequencing reads categorized in each of the artifact training data 3310A, non-artifact training data 33110B, and reference allele training data 3330. Each group of sequencing reads corresponds to the same position in the genome. Specifically, artifact features 3320 and non-artifact features 3325 are extracted from sequence reads in one, two, or all three of the artifact training data 3310A, non-artifact training data 3310B, and reference allele training data 3330. Examples of artifact features 3320 and non-artifact features 3325 include a statistical distance from edge feature, a significance score feature, and an allele fraction feature. Each of these features are described in further detail below in relation to FIGS. 29E-29G.

FIG. 29E is an example depiction of a process for extracting a statistical distance from edge feature according to one embodiment. Here, the edge filter 1250 extracts the artifact and non-artifact statistical distance from edge 3322A and 3322B features from a group of sequence reads in the artifact training data 3310A and a group of sequence reads in the non-artifact training data 3310B, respectively. Each statistical distance from edge 3322A and 3322B feature can represent one of a mean, median, or mode of the distance (e.g., number of nucleotide base pairs) between alternative alleles 3375 on sequence reads and the corresponding edge of sequence reads. More specifically, artifact statistical distance from edge 3322A represents the combination of edge distances 3350A (see FIG. 29B) across sequence reads in a group of the artifact training data 3310A. Similarly, non-artifact statistical distance from edge 3322B represents the combination of edge distances 3350B (see FIG. 29C) across sequence reads in a group of the artifact training data 3310B.

FIG. 29F is an example depiction of a process for extracting a significance score feature according to one embodiment. The edge filter 1250 extracts the artifact significance score 3323A feature from a combination of a group of sequence reads in the artifact training data 3310A and a group of sequence reads in the reference allele training data 3330. Similarly, the edge filter 1250 extracts non-artifact significance score 3323B feature from a combination of a group of sequence reads in the non-artifact training data 3310B and a group of sequence reads in the reference allele training data 3330. Generally, the groups of sequence reads from the artifact training data 3310A, non-artifact training data 33108, and reference allele training data 3330 correspond to a common position on the genome. Therefore, for each position, there can be an artifact significance score 3323A and a non-artifact significance score 3323B for that position. Although the subsequent description refers to the process of extracting an artifact significance score 3323A, the same description applies to the process of extracting a non-artifact significance score 3323B.

The artifact significance score 3323A feature is a representation of whether the location of alternative alleles 3375A (e.g., in terms of distance from edge of a sequence read or another measure) on a group of sequence reads in the artifact training data 3310A is sufficiently different, to a statistically significant degree, from the location of reference alleles 3380 on a group of sequencing reads in the reference allele training data 3330. Specifically, artifact significance score 3323A is a comparison between edge distances 3350A of alternative alleles 3375A (see FIG. 29B) in the artifact training data 3310A and edge distances 3350C of reference alleles 3380 (see FIG. 29I)) in the reference allele training data 3330.

In various embodiments, the edge filter 1250 performs a statistical significance test for the comparison between edge distances. As one example, the statistical significance test is a Wilcoxon rank-sum test. Here, the edge filter 1250 assigns each sequence read in the artifact training data 33110A and each sequence read in the reference allele training data 3330 a rank depending on the magnitude of each edge distance 3350A and 3350C, respectively. For example, a sequence read that has the greatest edge distance 3350A or 3350C can be assigned the highest rank (e.g., rank=1), the sequence read that has the second greatest edge distance 3350A or 3350C can be assigned the second highest rank (e.g., rank=2), and so on. The edge filter 1250 compares the median rank of sequence reads in the artifact training data 3310A to the median rank of sequence reads in the reference allele training data 3330 to determine whether the locations of alternative alleles 3375 in the artifact training data 3310A significantly differ from locations of reference alleles 3380 in the reference allele training data 3330A. As an example, the comparison between the median ranks can yield a p-value, which represents a statistical significance score as to whether the median ranks are significantly different. In various embodiments, the artifact significance score 3223A is represented by a Phred score, which can be expressed as:

Phred Score=−10P

where P is the p-value score. Altogether, a low artifact significance score 3323A signifies that the difference in median ranks is not statistically significant whereas a high artifact significance score 3323A signifies that the difference in median ranks is statistically significant.

FIG. 29G is an example depiction of a process for extracting an allele fraction feature according to one embodiment. The allele fraction feature refers to the allele fraction of alternative alleles 3375A or 3375B. Specifically, artifact allele fraction 3324A refers to the allele fraction of alternative allele 3375A (see FIG. 29B) whereas non-artifact allele fraction 3324B refers to the allele fraction of alternative allele 3375B (see FIG. 29C). The allele fraction represents the fraction of sequence reads corresponding to the position in the genome that includes the alternative allele. For example, there may be X total sequence reads in the artifact training data 3310A that include the alternative allele 3375A. There may also be Y total sequence reads in the non-artifact training data 33110B that include the alternative allele 3375B. Additionally, there may be Z total sequence reads in the reference allele training data 3330 with the reference allele. Therefore, the artifact allele fraction 3324A of the alternative allele 3375A can be denoted as

$$Allele Fraction ( 3324 A ) = \frac{X}{X + Y + Z} .$$

Additionally, the non-artifact allele fraction 3324B of the alternative allele 3375B can be denoted as

$$Allele Fraction ( 3324 B ) = \frac{Y}{X + Y + Z} .$$

Returning, to FIG. 29A, the edge filter 1250 compiles extracted artifact features 3320 from groups of sequence reads across various positions of the genome to generate an artifact distribution 3340. Additionally, the edge filter 1250 compiles extracted non-artifact features 3325 from groups of sequence reads across various positions of the genome to generate a non-artifact distribution 3345. FIG. 29A depicts one particular embodiment where three different features 3320A are used to generate an artifact distribution 3340 and three different features 3 320B are used to generate a non-artifact distribution 3345. In other embodiments, fewer or more of each type of feature 3320A or 3320B are used to generate an artifact distribution 3340 or non-artifact distribution 3345.

FIGS. 29H and 29I depict example distributions used for identifying edge variants, according to various embodiments. Specifically, FIG. 29H depicts a distribution 2340 or 2345 generated from one type of artifact feature 3320 or non-artifact feature 3325. Although FIG. 29G depicts a normal distribution for sake of illustration, in practice, distributions 2340 and 2345 will vary depending on the values of the feature 3320 or 3325.

In another example, the edge filter 1250 can use multiple artifact features 3320 or non-artifact features 3325 to generate a single distribution 3340 or 3345. For example, FIG. 29I depicts a distribution 2340 or 2345 generated from two types of artifact features 3320 or two types of non-artifact features 3325. Here, the distribution 2340 or 2345 describes a relationship between a first feature and a second feature. In further embodiments, a distribution 2340 or 2345 can represent relationships between three or more types of artifact features 3320 or non-artifact features 3325.

### X. B. Example Determining of a Sample-Specific Rate for Identifying Edge Variants

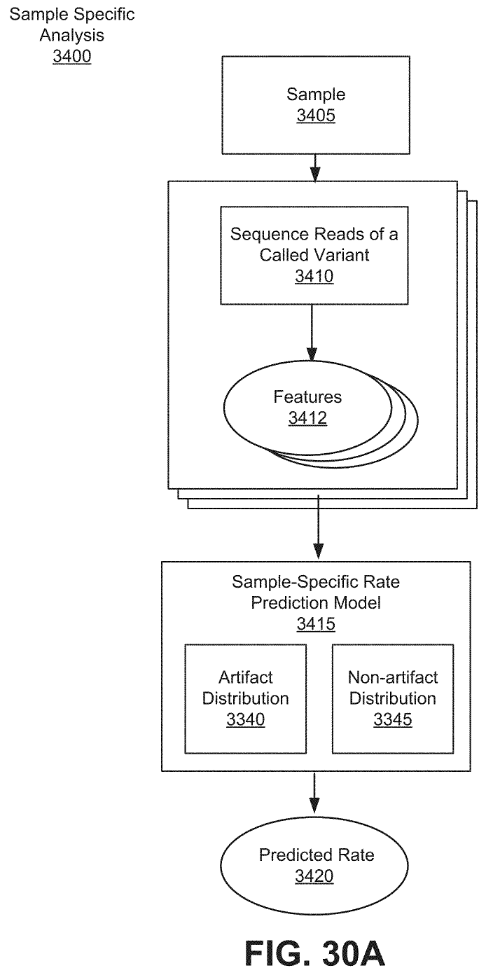

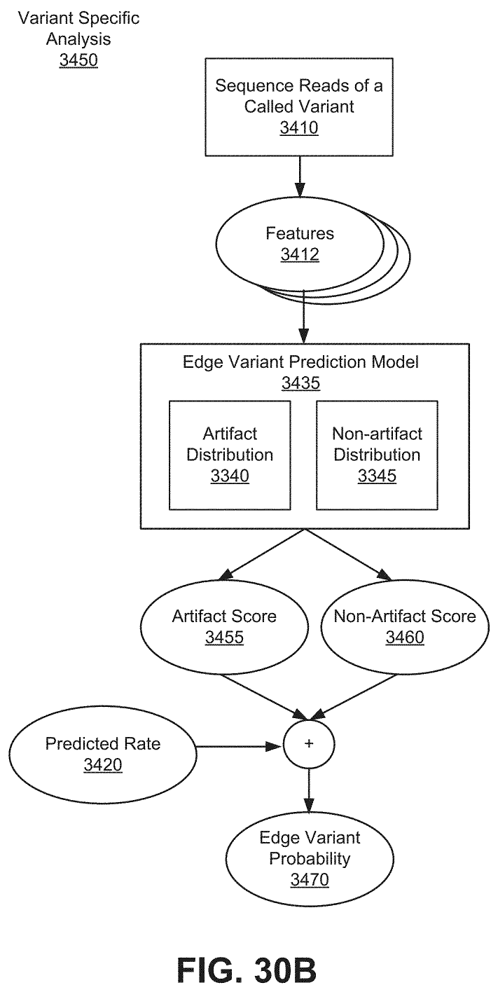

FIG. 30A depicts a block diagram flow process 3400 for determining a sample-specific predicted rate according to some embodiments. Generally, the edge filter 1250 conducts a sample-wide analysis of called variants in the sample 3405 to determine the predicted rate 3420 that is specific for the sample 3405. In other words, the process 3400 shown in FIG. 30A can be conducted once for each sample 3405.

Sequence reads of a called variant 3410 are obtained from a sample 3405. As described above in relation to FIG. 13, the steps for identifying called variants from a sample 3405 can include one or more steps of the method 1300. Generally, the sequence reads of a called variant 3410 refer to a group of sequence reads that cross over the position in the genome to which the called variant corresponds.

For each called variant, the edge filter 1250 extracts features 3412 from the sequence reads of the called variant 3410. Each feature 3412 extracted from sequence reads of a called variant 3410 can be a statistical distance from edge of alternative alleles in the sequence reads, an allele fraction of an alternative allele, a significance score, another type of feature, or some combination thereof. The edge filter 1250 applies features 3412 extracted across called variants of the sample 3405 as input to a sample-specific rate prediction model 3415 (e.g., one of the models 1225 shown in FIG. 12) that determines a predicted rate 3420 for the sample 3405. The predicted rate 3420 for the sample 3405 refers to an estimated proportion of called variants that are edge variants. In various embodiments, the predicted rate 3420 is a value between 0 and 1, e.g., inclusive.

As shown in FIG. 30A, the sample-specific rate prediction model 3415 uses both the previously generated artifact distribution 3340 and non-artifact distribution 3345. The sample-specific rate prediction model 3415 determines the predicted rate 3420 by analyzing the features 3412 extracted from sequence reads of called variants in the sample 3405 in view of the artifact distribution 3340 and non-artifact distribution 3345. As an example, the sample-specific rate prediction model 3415 performs a goodness of fit to determine a predicted rate 3420 that explains the observed features 3412, given the artifact distribution 3340 and non-artifact distribution 3345. In one example implementation, the sample-specific rate prediction model 3415 performs a maximum likelihood estimation to estimate the predicted rate 3420 that maximizes the likelihood of observing the features 3412 in view of the artifact distribution 3340 and non-artifact distribution 3345. However, other implementations may use other processes.

In some embodiments, the likelihood equation for the estimation can be expressed as:

L(w|x)=w*(L(x)|d₁)+(1−w)*(L(x|d₂) (1)

where w is the predicted rate 3420, x represents the features 3412, d₁ represents the artifact distribution 2340, and d₂ represents the non-artifact distribution 3345. In other words, Equation 1 is the weighted sum of a likelihood of observing the features 3412 in view of the artifact distribution 3340 and a likelihood of observing the features 3412 in view of the non-artifact distribution 3345. Therefore, the maximum likelihood estimation determines the predicted rate 3420 (e.g., rate w) that maximizes this overall likelihood given a certain set of conditions.

As shown in FIG. 30A, the edge filter 1250 can extract multiple features 3412 from sequence reads of a called variant 3410 and provide the features 3412 to the rate prediction model 3415. For example, there may be three types of features (e.g., statistical distance from edge of alternative alleles in the sequence reads, an allele fraction of an alternative allele, or a significance score). Generalizing further, assuming that n different types of features 3412 (e.g., x₁, x₂, . . . xₙ) are provided to the rate prediction model 3415, Equation 1 can be expressed as:

$$L ( w \vert {x}_{1} , {x}_{2} … {x}_{n} ) = w * ( {\prod }_{1}^{n} L ( {x}_{n} ) \vert {d}_{1} ) + ( 1 - w ) * ( {\prod }_{1}^{n} L ( {x}_{n} ) \vert {d}_{2} ) \qquad ( 2 )$$

Altogether, responsive to determining that the distribution of features 3412 extracted from sequence reads of the called variants in the sample 3405 are more similar to the artifact distribution 3340 than the non-artifact distribution 3345, the rate prediction model 3415 determines a high predicted rate 3420, which indicates that a high estimated proportion of called variants are likely edge variants. Alternatively, responsive to determining that the distribution of features 3412 extracted from sequence reads of variants in the sample 3405 are more similar to the non-artifact distribution 3345 than the artifact distribution 3340, the rate prediction model 3415 determines a low predicted rate 3420, which indicates that a low estimated proportion of called variants are likely edge variants. As discussed below, the predicted rate 3420 can be used to control for the level of “aggressiveness” in which edge variants are identified in a sample. Therefore, a sample that is assigned a high predicted rate 3420 can be aggressively filtered (e.g., using broader criteria to filter out a greater number of possible edge variants) whereas a sample that is assigned a low predicted rate 3420 can be less aggressively filtered.

### X. C. Example Variant Specific Analysis for Identifying an Edge Variant

FIG. 30B depicts the application of an edge variant prediction model 3435 for identifying edge variants according to some embodiments. In a variant specific analysis 3450, the edge filter 1250 analyzes sequence reads of a called variant 3410 to determine whether the called variant is an edge variant. The process depicted in FIG. 30B can be conducted for each called variant or a subset of called variants that were detected for a single sample 3405.

In some embodiments, the edge filter 1250 filters called variants based on a type of mutation of the called variant. Here, a called variant that is not of the C>T or G>A mutation type can be automatically characterized as a non-edge variant. Alternatively or additionally, any called variant that is of the C>T or G>A is further analyzed in the subsequent steps described hereafter.

As shown in FIG. 30B, the edge filter 1250 extracts features 3412 from the sequence reads of a called variant 3410. The extracted features 3412 of the sequence reads of a called variant 3410 can be the same features 3412 extracted from the sequence reads of a called variant 3410 as shown in FIG. 30A. Namely, the features 3412 can be one or more of: a statistical distance from edge of alternative alleles in the sequence reads, an allele fraction of an alternative allele, or a significance score, among other types of features.

The edge filter 1250 provides the extracted features 3412 as input to the edge variant prediction model 3435 (e.g., one of the models 1225 shown in FIG. 12). As shown in FIG. 30B, the edge variant prediction model 3435 uses both the previously generated artifact distribution 3340 and the non-artifact distribution 3345. The edge variant prediction model 3435 generates multiple scores, such as an artifact score 3455 that represents a likelihood that the called variant is an edge variant as well as a non-artifact score 3460 that represents a likelihood that the called variant is a non-edge variant.

Specifically, the edge variant prediction model 3435 determines the probability of observing the features 3412 of the sequence reads of a called variant 3410 in view of the artifact distribution 3340 and the non-artifact distribution 3345. In some embodiments, the edge variant prediction model 3435 determines the artifact score 3450 by analyzing the features 3412 in view of the artifact distribution 2340 and determines the non-artifact score 3455 by analyzing the features 3412 in view of the non-artifact distribution 3345.

As a visual example, referring back to the example distribution shown in FIG. 29H, the edge variant prediction model 3435 identifies a probability based on where along the x-axis a feature 3412 falls. In this example, the identified probability can be the score, such as the artifact score 3450 or non-artifact score 3455, outputted by the edge variant prediction model 3435.

As shown in FIG. 30B, the edge filter 1250 combines the artifact score 3455 and non-artifact score 3460 with the sample-specific predicted rate 3420 (as described in FIG. 30A). The combination yields the edge variant probability 3470, which represents the likelihood that the called variant is a result of a processing artifact.

In some embodiments, edge variant probability 3470 can be expressed as the posterior probability of the called variant being an edge variant in view of the features 3412 extracted from sequence reads of the called variant 3410. The combination of the artifact score 3455, the non-artifact score 3460, and the sample-specific predicted rate 3420 can be expressed as:

$$Edge Variant Probability = \frac{w * artifact score}{( w * artifact score ) + ( 1 - w ) * ( nonartifact score )} \qquad ( 3 )$$

The edge filter 1250 can compare the edge variant probability 3470 against a threshold value. Responsive to determining that the edge variant probability 3470 is greater than the threshold value, the edge filter 1250 determines that the called variant is an edge variant. Responsive to determining that the edge variant probability 3470 is less than the threshold value, the edge filter 1250 determines that the called variant is a non-edge variant.

### X. D. Example Variant Specific Analysis for Identifying an Edge Variant

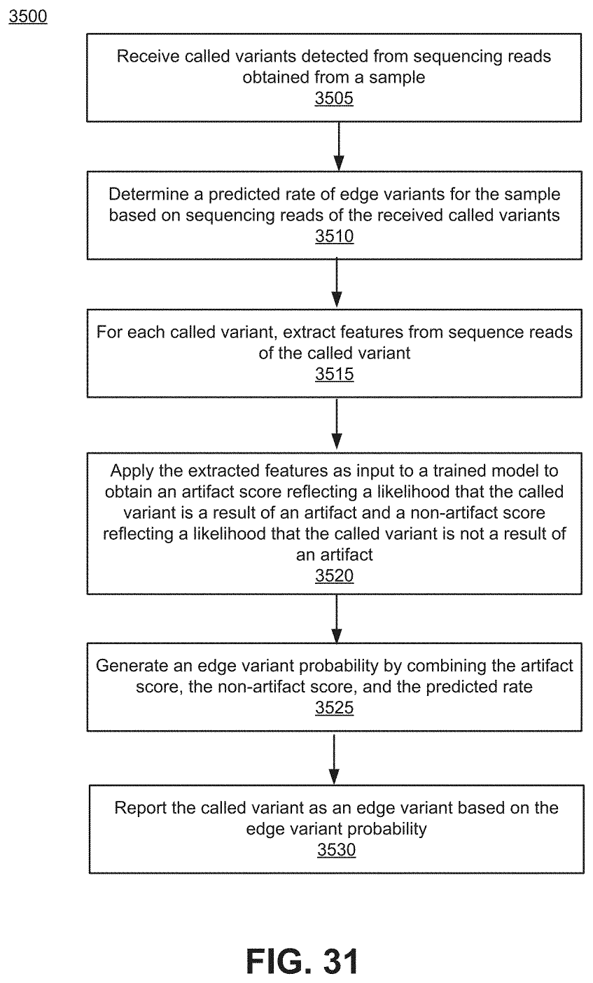

FIG. 31 depicts a flow process 3500 of identifying and reporting edge variants detected from a sample according to one embodiment. One or more steps of the process 3500 may be performed by components of the processing system 1200, e.g., the edge filter 1250 or one of the models 1225. Called variants from various sequencing reads are received (step 3505) from a sample. A sample-specific predicted rate is determined (step 3510) for the sample based on sequencing reads of the called variants from the sample. As one example, a predicted rate is determined by performing a maximum likelihood estimation. Here, the predicted rate is a parameter value that maximizes (e.g., given certain conditions) the likelihood of observing features 3412 of sequence reads of the called variants in view of previously generated distributions.

For each called variant, one or more features 3412 are extracted 3515 from the sequence reads of the variant. The extracted features 3412 are applied (step 3520) as input to a trained model 1225 to obtain an artifact score 3455. The artifact score 3455 represents a likelihood that the called variant is an edge variant (e.g., result of a processing artifact). The trained model 1225 further outputs a non-artifact score 3460 which represents a likelihood that the called variant is a non-edge variant (e.g., not a result of a processing artifact).

For each called variant, an edge variant probability 3470 is generated (step 3525) by combining the artifact score 3455 for the called variant, non-artifact score 3460 for the called variant, and the sample-specific predicted rate 3420. Based on the edge variant probability 3470, the called variant can be reported (step 3530) as an edge variant (e.g., variants that were called as a result of a processing artifact).

### XI. EXAMPLE VARIANT CALLER

### XI. A. Example Combination of Different Filters and Scoring

FIG. 32 is a flowchart of a method 4200 for processing candidate variants using different types of filters and models 1225 according to one embodiment. One or more steps of the method 4200 can be performed in conjunction with other methods described herein, or with another method. For example, the method 4200 can be performed as the filtering step 1320 of the method 1300 shown in FIG. 13 to identify and remove any false positives, e.g., before calling variants. The method 4200 can include different, additional, or fewer steps than those described in conjunction with FIG. 32 in some embodiments or perform steps in different orders than the order described in conjunction with FIG. 32. For instance, the method 4200 can filter using a joint model but not with edge filtering. As a different example, the method 4200 can perform edge filtering before filtering using the joint model. In some embodiments, one or more steps can be combined, e.g., the method 4200 includes filtering using the joint model and edge filtering in the same step.

At step 4210, the processing system 1200 uses at least one model 1225 to model noise of sequence reads of a nucleic acid sample, e.g., a cfDNA sample. The model 1225 can be a Bayesian hierarchical model as previously described with reference to FIGS. 14-19, which approximates expected noise distribution per position of the sequence reads. At step 4220, the processing system 1200 filters candidate variants from the sequence reads using a joint model 1225, e.g., as previously described with reference to FIGS. 20-25. In some embodiments, the processing system 1200 uses the joint model 1225 to determine whether a given candidate variant observed in the cfDNA sample is likely associated with a nucleotide mutation of a corresponding gDNA sample (e.g., from white blood cells).

At step 4230, the processing system 1200 filters the candidate variants using edge filtering, in some embodiments. In particular, the edge filter 1250 can use a sample-specific rate prediction model 3415 (see FIG. 30A) and an edge variant prediction model 3435 (see FIG. 30B) to determine how aggressively to filter the sample to remove edge variants, e.g., as previously described with reference to FIGS. 29A-I, 30A-B, and 31. In some embodiments, the score engine 1235 uses the models for edge filtering to analyze and assign a support score to each candidate variant (or called variant), where the support score represents a level of confidence that the candidate variant is a non-edge variant. The edge filter 1250 keeps a candidate variant associated with a support score that greater than a threshold score, whereas the edge filter 1250 filters out candidate variants associated with a support score less than (or equal to) the threshold score. In some embodiments, the score engine 1235 generates the support score for a candidate variant based on prior knowledge about the candidate variant and/or systematic errors observed in a set of healthy samples for that chromosome/position. In some scenarios, the support score can be determined based on sequencing depth of a target region including the candidate variant, and the threshold score can be based on an average sequencing depth of the target region in a set of previously sequenced samples (e.g., reference data).

As described above regarding the edge filter 1250, sequence reads obtained from sample can include both sequence reads that include an alternative allele as well as sequence reads that include a reference allele. Specifically, given the collection of candidate variants for a sample, the edge filter 1250 can perform a likelihood estimation to determine a predicted rate of edge variants in the sample. Given certain conditions of the sample, the predicted rate can best explain the observed collection of candidate variants for the sample in view of two distributions. One distribution describes features of known edge variants whereas another trained distribution describes features of known non-edge variants. The predicted rate is a sample-specific parameter that controls how aggressively the sample is analyzed to identify and filter edge from the sample. Edge variants of the sample are filtered and removed, leaving non-edge variants for subsequent consideration (e.g., for determination of presence/absence of cancer or likelihood of cancer or other disease).

At step 4240, the non-synonymous filter 1260 can optionally filter the candidate variants based on non-synonymous mutations, in some embodiments. In contrast to a synonymous mutation, a non-synonymous mutation of a nucleic acid sequence results in a change in the amino acid sequence of a protein associated with the nucleic acid sequence. For instance, non-synonymous mutations can alter one or more phenotypes of an individual or cause (or leave more vulnerable) the individual to develop cancer, cancerous cells, or other types of diseases. In some embodiments, the non-synonymous filter 1260 determines that a candidate variant should result in a non-synonymous mutation by determining that a modification to one or more nucleobases of a trinucleotide would cause a different amino acid to be produced based on the modified trinucleotide. In some embodiments, the non-synonymous filter 1260 keeps candidate variants associated with non-synonymous mutations and filters out other candidate variants associated with synonymous mutations because the former group of candidate variants are more likely to have a functional impact on an individual.

### XII. ADDITIONAL CONSIDERATIONS

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules can be embodied in software, firmware, hardware, or any combinations thereof.

Any of the steps, operations, or processes described herein can be performed or implemented with one or more hardware or software modules, alone or in combination with other devices. In some embodiments, a software module is implemented with a computer program product including a computer-readable non-transitory medium containing computer program code, which can be executed by a computer processor for performing any or all of the steps, operations, or processes described.

Embodiments of the invention can also relate to a product that is produced by a computing process described herein. Such a product can include information resulting from a computing process, where the information is stored on a non-transitory, tangible computer readable storage medium and can include any embodiment of a computer program product or other data combination described herein

Finally, the language used in the specification has been principally selected for readability and instructional purposes, and it may not have been selected to delineate or circumscribe the inventive subject matter, it is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method for detecting positive, neutral, or negative selection at a locus, the method comprising:
(a) obtaining a test sample from a subject, wherein the test sample comprises a plurality of cell-free nucleic acids;
(b) preparing a sequencing library from the plurality of cell-free nucleic acids;
(c) sequencing the library to obtain a plurality of sequence reads, wherein the sequence reads are derived from the plurality of cell-free nucleic acids;
(d) analyzing the plurality of sequence reads to detect and quantify one or more somatic mutations at the locus;
(e) determining a selection coefficient for the locus; and
(f) comparing the selection coefficient determined for the locus with a threshold value and detecting positive, neutral, or negative selection at the locus based on the comparison.

2. The method of claim 1, wherein the one or more somatic mutations are white blood cell matched somatic mutations, the method further comprising:
(a) obtaining white blood cells from the test sample;
(b) isolating nucleic acids from the white blood cell and preparing a sequencing library from the white blood cell nucleic acids;
(c) sequencing the library to obtain a plurality of sequence reads derived from the white blood cell nucleic acids;
(d) analyzing the plurality of sequence reads derived from the white blood cell nucleic acids to detect and quantify one or more white blood cell derived somatic mutations; and
(e) comparing the one or more cell-free nucleic acid detected somatic mutation and the one or more white blood cell derived somatic mutations to identify one or more white blood cell matched somatic mutations.

3. The method of claim 1, wherein analyzing the plurality of sequence reads to detect and quantify the one or more somatic mutations further comprises applying a noise model.

4. The method of claim 1, wherein analyzing the plurality of sequence reads further comprises applying a read mis-mapping model.

5. The method of claim 2, wherein the one or more somatic mutations are detected from joint modeling or mixture modeling both the one or more cell-free nucleic acid somatic mutation and the one or more white blood cell derived somatic mutations.

6. The method of claim 1, wherein the selection coefficient comprises a ratio between the rate of non-synonymous substitutions per non-synonymous site and the rate of synonymous substitutions per synonymous site.

7. The method of claim 1, wherein when the threshold is greater than 1 with a q-value less than 0.05 positive selection is detected, and wherein when the threshold is less than 1 with a q-value less than 0.05 negative selection is detected.

8. (canceled)

9. The method of claim 1, wherein the one or more somatic mutations comprise one or more single-nucleotide variants.

10. The method of claim 1, wherein the one or more somatic mutations comprise one or more nonsynonymous mutations and the selection coefficient is determined based on the one or more nonsynonymous mutations.

11. The method of claim 1, wherein the one or more somatic mutations comprise one or more missense mutations and the selection coefficient is determined based on the one or more missense mutations.

12. The method of claim 1, wherein the one or more somatic mutations comprise one or more nonsense mutations and the selection coefficient is determined based on the one or more nonsense mutations.

13. The method of claim 1, wherein the one or more somatic mutations comprise one or more truncating mutations and the selection coefficient is determined based on the one or more truncating mutations.

14. The method of claim 9, the method further comprising:
identifying the one or more somatic mutations as one or more tumor suppressors if the locus includes at least one nonsense mutation, or
identifying the one or more somatic mutations as one or more oncogenes if the locus includes at least one nonsense mutation and at least one missense mutation.

15. (canceled)

16. The method of claim 1, wherein the one or more somatic mutations comprise one or more essential splice site mutations and the selection coefficient is determined based on the one or more essential splice site mutations.

17. The method of claim 1, wherein the one or more somatic mutations are detected in an oncogene and the selection coefficient is determined for the oncogene.

18. The method of claim 1, wherein the one or more somatic mutations are detected in a tumor suppressor gene and the selection coefficient is determined for the tumor suppressor gene.

19. The method of claim 1, wherein the one or more somatic mutations comprise one or more insertions and/or deletions.

20. The method of claim 1, wherein the one or more somatic mutations occur within one or more genes selected from the group consisting of DNMT3A, TET2, CHEK2, CBL, TP53, ASXL1, PPM1D, SF3B1, ARID2, ATM, DNMT3B, SH2B3, RAD21, SRSF2, JAK2, KMT2C, MGA, KDR, KRAS, MST1, ERRFI1, CCND2, EWSR1, MYD88, CDKN1B, and any combination thereof.

21. The method of claim 1, wherein the sequencing comprises targeted sequencing, and the targeted sequencing comprises a targeted enrichment step prior to sequencing, and wherein the targeted enrichment step comprises an enrichment panel comprising from about 10 to about 10,000 targeted genes.

22. The method of claim 1, wherein the selection coefficient determined at the locus is based on one or more somatic mutations detected at the loci have an allele frequency of from about 0.01% to about 35%.

23. The method of claim 1, wherein the cell-free nucleic acids comprise cell-free DNA.

24. The method of claim 1, wherein the cell-free nucleic acids comprise cell-free RNA.

25. The method of claim 1, wherein the method further comprises at least one of assessing a risk of developing a disease state, detecting a disease state, and diagnosing a disease state based on the identification of one or more somatic mutations at the locus, wherein the disease state is a cardiovascular disease or a cancer.

26. (canceled)

27. (canceled)

28. The method of claim 1, wherein the locus detected as having positive selection is identified as a target for a therapeutic treatment.

29. The method of claim 1, wherein the one or more detected somatic mutations are at locus targeted by immuno oncology therapy, targeted therapy, or synthetic lethality therapy, further wherein the detection of negative selection after therapeutic treatment with treatment with the immuno oncology therapy, targeted therapy, or synthetic lethality therapy in an indicator of treatment response.

30. (canceled)

31. A computer-implemented method for detecting positive, neutral, or negative selection at a locus, the method comprising:
receiving a first data set in a computer comprising a processor and a computer-readable medium, wherein the first data set comprises a plurality of sequence reads obtained by sequencing a plurality of cell-free nucleic acids in a test sample from a subject, and wherein the computer-readable medium comprises instructions that, when executed by the processor, cause the computer to:
analyze the data set to detect and quantifying one or more somatic mutations at the locus;
calculate a selection coefficient for the locus; and
comparing the selection coefficient calculated for the locus with a threshold value to detect positive, neutral, or negative selection at the locus.

32. An electronic device, comprising:
one or more processors;
memory; and
one or more programs, wherein the one or more programs are stored in the memory and configured to be executed by the one or more processors, the one or more programs including instructions comprising:
(a) obtaining a test sample from a subject, wherein the test sample comprises a plurality of cell-free nucleic acids;
(b) preparing a sequencing library from the plurality of cell-free nucleic acids;
(c) sequencing the library to obtain a plurality of sequence reads, wherein the sequence reads are derived from the plurality of cell-free nucleic acids;
(d) analyzing the plurality of sequence reads to detect and quantify one or more somatic mutations at the locus;
(e) determining a selection coefficient for the locus; and
(f) comparing the selection coefficient determined for the locus with a threshold value and detecting positive, neutral, or negative selection at the locus based on the comparison.

33-65. (canceled)
